(19) United States
(12) Patent Application Publication
(10) Pub. No.: US 2023/0109142 A1
(43) Pub. Date: Apr. 6, 2023

(54) CORONA VIRUS VACCINE

(71) Applicant: Immunor AS, Oslo (NO)

(72) Inventors: Andres Schjønhaug Susrud, Oslo (NO); Birger Sørensen, Oslo (NO); Samara Simha Mamidi, Lund (SE); Richard Escobar Johnsson, Lund (SE)

(73) Assignee: Immunor AS, Oslo (NO)

(21) Appl. No.: 17/904,053

(22) PCT Filed: Feb. 15, 2021
(86) PCT No.: PCT/EP2021/053668

§ 371 Date: Aug. 11, 2022

(30) Foreign Application Priority Data
Feb. 14, 2020 (EP) 20157450.6
Apr. 6, 2020 (EP) 20168251.5
Dec. 17, 2020 (EP) 20214862.3

(51) Int. Cl. A61K 39/215; C07K 14/005; C12N 7/00

(52) U.S. Cl. CPC A61K 39/215; C07K 14/005; C12N 7/00; C12N 2770/20022; C12N 2770/20034

(57) ABSTRACT

The present invention relates to the field of virus immunotherapy. In particular the present invention relates to novel peptides and methods for treatment, induction of immunity, prophylaxis and amelioration of a disease caused by virus infections with Corona virus, in particular Wuhan seafood market pneumonia virus isolate Wuhan-Hu-1.

### FIELD OF THE INVENTION

The present invention relates to the field of virus immunotherapy. In particular, the present invention relates to novel peptides and methods for treatment, induction of immunity, prophylaxis and amelioration of a disease caused by virus infections with Corona virus, in particular Wuhan seafood market pneumonia virus isolate Wuhan-Hu-1.

### BACKGROUND OF THE INVENTION

Coronaviruses (CoV) are single-stranded positive-sense RNA viruses that infect animals and humans. These are classified into 4 genera based on their host specificity: Alphacoronavirus, Betacoronavirus, Deltacoronavirus and Gammacoronavirus. There are seven known types of CoVs that includes 229E and NL63 (Genus Alphacoronavirus), OC43, HKU1, MERS and SARS (Genus Betacoronavirus). While 229E, NL63, OC43, and HKU1 commonly infect humans, the SARS and MERS outbreak in 2002 and 2012 respectively occurred when the virus crossed over from animals to humans causing significant mortality. In December 2019, another outbreak of coronavirus was reported from Wuhan, China that also transmitted from animals to humans. This new virus has been temporarily termed as 2019-novel Coronavirus (2019-nCoV) by the World Health Organization (WHO). While there are several hypotheses about the origin of 2019-nCoV, the source of this ongoing outbreak remains elusive.

Cases of mild to severe illness, and death from the infection have been reported from Wuhan. This outbreak has spread rapidly distant nations including France, Australia and USA among others. The number of cases within and outside China is increasing steeply. Our current understanding is limited to the virus genome sequences and modest epidemiological and clinical data. The transmission patterns of 2019-nCoV is similar to patterns of transmission documented in the previous outbreaks including by bodily or aerosol contact with persons infected with the virus.

There is an urgent need for a detailed analysis of the available 2019-nCoV sequences, which provides important clues for an optimal vaccine candidate that may help manage the ongoing outbreak.

### OBJECT OF THE INVENTION

It is an object of embodiments of the invention to provide an efficient vaccine candidate against coronaviruses and, in particular, against Wuhan seafood market pneumonia virus isolate Wuhan-Hu-1.

It is a further object of embodiments to provide insight in the structure of the protein sequence of these corona viruses for the provision of alternative or even better vaccine candidates.

It is a further object of the invention to provide such vaccine candidates which avoid human or human-like amino acid sequences.

### SUMMARY OF THE INVENTION

The present invention pertains to peptides and polypeptides promoting efficient activation of a humoral immune response against coronaviruses and, in particular, against Wuhan seafood market pneumonia virus isolate Wuhan-Hu-1, also known as Covid-19 virus and SARS-CoV-2.

The present invention pertains in particular to a peptide design promoting efficient activation of a humoral immune response against antigens contained within this peptide design as well as to a peptide design promoting uptake of peptide epitopes by antigen presenting cells (macrophages and dendritic cells) such that the epitopes can be correctly processed and presented in the context of HLA class I and II to stimulate both CD4+ and CD8+ T-lymphocytes. CD8+ T-lymphocytes with cytotoxic capacity will kill infected cells bearing the epitope of interest. CD4+ T-lymphocyte provide ‘help’ to sustain effective CD8+ T-lymphocyte responses and promote support for development of antibody humoral immune responses.

It has been found by the present inventor(s) that peptide constructs—amino acid sequences with a particular sequence and pattern, structure or scaffold design, or as multimeric, such as dimeric peptides of this design—have the ability to effectively elicit a humoral immune response in a subject in response to the administration of these peptides. Moreover, the peptide constructs are designed so as to avoid human and human-like amino acid sequences. Particularly contemplated are peptide constructs designed to elicit a humoral response against structurally or functionally important sites in native proteins of the virus. Without being limited to theory, structurally or functionally important sites in the spike protein include a receptor-binding domain for the human ACE-2 receptor (Angiotensin-converting enzyme 2 receptor) and/or the CD209 antigen, a ganglioside-binding domain, and a furin cleavage site (TMPRSS2 target).

The peptide constructs according to the present invention may be designed to be able to attach or bind to the cell surface. The peptide constructs or parts thereof may then be taken up by the antigen presenting cells (such as macrophages and dendritic cells) and stimulate helper T-cells in order to elicit efficient and long-lasting T-cell dependent B-cell activation. Alternatively, the B-cells themselves may provide for the induction of help to activate the B-cells.

The peptides according to the present invention should preferably be able to penetrate the cells and be used to load cells with an immunogenically effective amount of a peptide or fragments of this peptide that can be presented by macrophages and dendritic cells. Accordingly, these peptide constructs may elicit both a Cytotoxic T-lymphocyte immune (CTL) response and/or a humoral immune response.

These and other features of the invention are described in more detail below.

### BRIEF DESCRIPTION OF THE FIGURES

FIG. 1 shows a structural representation of the SARS-CoV-2 spike protein (e.g., SEQ ID NO:1), with circles indicating surface sites of interest. The top circle indicates a ganglioside-binding domain comprising the segments HVSGTNGTKRFD identified as position 69 to 80 of SEQ ID NO:1, HRSYLTPGDSSSGWTAGAA identified as position 245 to 263 of SEQ ID NO:1, and VYYHKNNKSWMESEFRVYSSANN identified as position 143 to 165 of SEQ ID NO:1. The middle circle indicates a furin cleavage site (TMPRSS2 target) comprising the segment TQTNSPSGAGVAS (SEQ ID NO: 959) alt. TQTNSPRRARSVAS identified as position 676 to 689 of SEQ ID NO:1. The bottom circle indicates a receptor binding domain for ACE2 and/or CD209(L)), comprising the segments ISTEIYQAGSTPCNGVEGFNCY identified as position 468 to 489 and KVGGNY (SEQ ID NO:35) identified as position 444 to 449 of SEQ ID NO: 1.

### DETAILED DESCRIPTION OF THE INVENTION

In a first aspect the present invention relates to a monomeric peptide consisting of at least 5, such as at least 6 consecutive amino acids of the sequence of amino acids STPCNGVEGFNC identified as position 477-488 of SEQ ID NO:1; or a variant thereof containing one, two, three, or four amino acid substitutions, which variant has not more than one amino acid substitution per three, such as per four, such as per five, such as per six consecutive amino acids of said sequence STPCNGVEGFNC. In a further aspect the present invention relates to a monomeric peptide consisting of at least 5, such as at least 6 consecutive amino acids of the sequence of amino acids PCNGVEGFNCYFP identified as position 479-491 of SEQ ID NO:1; or a variant thereof containing one, two, three, four or five amino acid substitution, which variant has not more than one amino acid substitution per three, such as per four, such as per five, such as per six consecutive amino acids of said sequence STPCNGVEGFNC. In some specific embodiments, this peptide may consist of a sequence selected from SEQ ID NO: 38, SEQ ID NO: 39, SEQ ID NO: 40, or SEQ ID NO: 41, or a variant thereof containing one or two amino acid substitutions, or one amino acid deletion. In other embodiments this peptide has one or more amino acid substitutions selected from S to T in position 477 of SEQ ID NO: 1; T to S in position 478 of SEQ ID NO:1; N to Q in position 481 of SEQ ID NO:1; V to any one of L, I, A or norleucin in position 483 of SEQ ID NO:1; E to D in position 484 of SEQ ID NO:1; F to Yin position 486 of SEQ ID NO:1; or N to Q or Sin position 487 of SEQ ID NO:1. In other embodiments this peptide has G to P in position 482 of SEQ ID NO:1. In other embodiments this peptide has V to A or L in position 483 of SEQ ID NO:1. In other embodiments this peptide has E to D in position 484 of SEQ ID NO:1. In other embodiments this peptide has F to D in position 486 of SEQ ID NO:1. In other embodiments, this peptide has a C to S substitution in position 480 of SEQ ID NO:1. In other embodiments, this peptide has an E to K substitution in position 484 of SEQ ID NO:1. In some embodiments, the amino acid at position 484 in SEQ ID NO:1 is E, K, or D, such as E or K, such as K. It is to be understood that such monomeric peptide consisting of at least 5, such as at least 6 consecutive amino acids of the sequence of amino acids STPCNGVEGFNC identified as position 477-488 of SEQ ID NO:1 is no longer than 12 amino acids. Accordingly, in embodiments where a monomeric polypeptide of 10-80 amino acids comprises a monomeric polypeptide according to this aspect, the monomeric peptide may be linked via a peptide bond at its N-terminal, C-terminal, or both N- and C-terminal, to a heterologous amino acid sequence, i.e., an amino acid sequence to which it is not linked in SEQ ID NO:1.

In a second aspect the present invention relates to a monomeric peptide consisting of at least 5, such as at least 6 consecutive amino acids of the sequence of amino acids LDSKVGGNY identified as position 441-449 of SEQ ID NO:1; or a variant thereof containing one, two, or three amino acid substitutions, which variant has not more than one amino acid substitution per three, such as per four, such as per five, such as per six consecutive amino acids of said sequence LDSKVGGNY. In some specific embodiments, this peptide may consist of a sequence selected from SEQ ID NO: 33, SEQ ID NO: 34, or SEQ ID NO: 35, or a variant thereof containing at one or two amino acid substitutions, or one amino acid deletion. In other embodiments this peptide has one or more amino acid substitutions selected from D to E in position 442 of SEQ ID NO:1; S to T in position 443 of SEQ ID NO:1; K to any one of R or homoarginine in position 444 of SEQ ID NO:1; V to any one of L, I, A or norleucine in position 445 of SEQ ID NO:1; N to Q in position 448 of SEQ ID NO:1; or Y to F in position 449 of SEQ ID NO:1. It is to be understood that such monomeric peptide consisting of at least 5, such as at least 6 consecutive amino acids of the sequence of amino acids LDSKVGGNY identified as position 441-449 of SEQ ID NO:1 is no longer than 9 amino acids.

In a third aspect the present invention relates to a monomeric peptide consisting of at least 5, such as at least 6 consecutive amino acids of the sequence of amino acids FQPTNGVGYQP identified as position 497-507 of SEQ ID NO:1; or a variant thereof containing one, two, or three amino acid substitution, which variant has not more than one amino acid substitution per three, such as per four, such as per five, such as per six consecutive amino acids of said sequence FQPTNGVGYQP. In some specific embodiments, this peptide may consist of a sequence selected from SEQ ID NO: 42, SEQ ID NO: 43, or SEQ ID NO: 44, or a variant thereof containing one or two amino acid substitutions, or one amino acid deletion. In other embodiments this peptide has one or more amino acid substitutions selected from F to Y in position 497 of SEQ ID NO:1; Q to N in position 498 of SEQ ID NO:1; T to S in position 500 of SEQ ID NO:1; N to Q in position 501 of SEQ ID NO:1; V to any one of L, I, A or norleucine in position 503 of SEQ ID NO:1; Y to F in position 505 of SEQ ID NO:1; or Q to N in position 506 of SEQ ID NO:1. In other embodiments, this peptide has an N to Y substitution in residue 501 of SEQ ID NO:1. It is to be understood that such monomeric peptide consisting of at least 5, such as at least 6 consecutive amino acids of the sequence of amino acids FQPTNGVGYQP identified as position 497-507 of SEQ ID NO:1 is no longer than 11 amino acids. In some embodiments, the amino acid at position 501 in SEQ ID NO:1 is Y or N, such as Y.

In a fourth aspect the present invention relates to a monomeric peptide consisting of at least 5, such as at least 6 consecutive amino acids of the sequence of amino acids FKCYGVSPTKLNDS identified as corresponding to position 377-391 of SEQ ID NO:1; or a variant thereof containing one, two, or three amino acid substitution, which variant has not more than one amino acid substitution per three, such as per four, such as per five, such as per six consecutive amino acids of said sequence FKCYGVSPTKLNDS. In some embodiments the present invention relates to a monomeric peptide consisting of at least 5, such as at least 6 consecutive amino acids of the sequence of amino acids FKCYGVSPTKLND identified as position 377-390 of SEQ ID NO:1; or a variant thereof containing one, two, or three amino acid substitution, which variant has not more than one amino acid substitution per three, such as per four, such as per five, such as per six consecutive amino acids of said sequence FKCYGVSPTKLND. In some specific embodiments, this peptide may consist of a sequence selected from SEQ ID NO: 20, SEQ ID NO: 21, SEQ ID NO: 22, SEQ ID NO: 23, SEQ ID NO: 24, or SEQ ID NO: 25, or a variant thereof containing one or two amino acid substitutions, or one amino acid deletion. In some specific embodiments, this peptide may have an amino acid substitution of C to S in position 379 of SEQ ID NO:1. It is to be understood that such monomeric peptide consisting of at least 5, such as at least 6 consecutive amino acids of the sequence of amino acids FKCYGVSPTKLND identified as position 377-390 of SEQ ID NO:1 is no longer than 13 amino acids. It is to be understood that such monomeric peptide consisting of at least 5, such as at least 6 consecutive amino acids of the sequence of amino acids FKCYGVSPTKLNDS identified as position 377-391 of SEQ ID NO:1 is no longer than 14 amino acids.

In a fifth aspect the present invention relates to a monomeric peptide consisting of at least 5, such as at least 6 consecutive amino acids of the sequence of amino acids PLSETKCTLKS identified as position 295-305 of SEQ ID NO:1; or a variant thereof containing one, two, or three amino acid substitution, which variant has not more than one amino acid substitution per three, such as per four, such as per five, such as per six consecutive amino acids of said sequence PLSETKCTLKS. In some specific embodiments, this peptide may consist of a sequence selected from SEQ ID NO: 109, SEQ ID NO: 110, or SEQ ID NO: 12, or a variant thereof containing one or two amino acid substitutions, or one amino acid deletion. It is to be understood that such monomeric peptide consisting of at least 5, such as at least 6 consecutive amino acids of the sequence of amino acids PLSETKCTLKS identified as position 295-305 of SEQ ID NO:1 is no longer than 11 amino acids.

In a sixth aspect the present invention relates to a monomeric peptide consisting of at least 5, such as at least 6 consecutive amino acids of the sequence of amino acids PATVCGPKKSTNLVKNKCV identified as position 521-539 of SEQ ID NO:1; or a variant thereof containing one, two, or three amino acid substitution, which variant has not more than one amino acid substitution per three, such as per four, such as per five, such as per six consecutive amino acids of said sequence PATVCGPKKSTNLVKNKCV. In some specific embodiments, this peptide may consists of a sequence selected from SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 289, or SEQ ID NO: 290, or a variant thereof containing one or two amino acid substitutions, or one amino acid deletion. In some specific embodiments, this peptide may consists of a sequence which peptide has one or more amino acid substitutions selected from C to T in position 525 of SEQ ID NO:1; C to S in position 538 of SEQ ID NO:1; C to S in position 525 of SEQ ID NO:1; and/or C to T in position 538 of SEQ ID NO:1. It is to be understood that such monomeric peptide consisting of at least 5, such as at least 6 consecutive amino acids of the sequence of amino acids PATVCGPKKSTNLVKNKCV identified as position 521-539 of SEQ ID NO:1 is no longer than 19 amino acids.

In a seventh aspect the present invention relates to a monomeric peptide consisting of 6-9 consecutive amino acids of SEQ ID NO:1, wherein the monomeric peptide comprises a sequence of amino acids as defined in any one of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 36, SEQ ID NO: 37, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, or SEQ ID NO: 288, SEQ ID NO: 289, or SEQ ID NO: 290, or a variant thereof comprising one, two or three amino acid substitutions, or one amino acid deletion.

In an eighth aspect the present invention relates to a monomeric peptide consisting of a sequence of amino acids as defined in any one of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 36, SEQ ID NO: 37, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, or SEQ ID NO: 288, SEQ ID NO: 289, or SEQ ID NO: 290, or a variant thereof containing one or two amino acid substitutions, or one amino acid deletion.

In a ninth aspect the present invention relates to a monomeric peptide consisting of 6-9 consecutive amino acids of SEQ ID NO:291, or a variant thereof containing one, two, or three amino acid substitutions.

In a tenth aspect the present invention relates to a monomeric peptide consisting of 6-9 consecutive amino acids of SEQ ID NO:291, wherein the monomeric peptide comprises a sequence of amino acids as defined in any one of SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, SEQ ID NO: 376, SEQ ID NO: 377, SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451, SEQ ID NO: 452, SEQ ID NO: 453, SEQ ID NO: 454, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, SEQ ID NO: 524, SEQ ID NO: 525, SEQ ID NO: 526, SEQ ID NO: 527, SEQ ID NO: 528, SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, SEQ ID NO: 588, SEQ ID NO: 589, SEQ ID NO: 590, SEQ ID NO: 591, SEQ ID NO: 592, SEQ ID NO: 593, SEQ ID NO: 594, SEQ ID NO: 595, SEQ ID NO: 596, SEQ ID NO: 597, SEQ ID NO: 598, SEQ ID NO: 599, SEQ ID NO: 600, SEQ ID NO: 601, SEQ ID NO: 602, SEQ ID NO: 603, SEQ ID NO: 604, SEQ ID NO: 605, SEQ ID NO: 606, SEQ ID NO: 607, SEQ ID NO: 608, SEQ ID NO: 609, SEQ ID NO: 610, SEQ ID NO: 611, SEQ ID NO: 612, SEQ ID NO: 613, SEQ ID NO: 614, SEQ ID NO: 615, SEQ ID NO: 616, SEQ ID NO: 617, SEQ ID NO: 618, SEQ ID NO: 619, SEQ ID NO: 620, SEQ ID NO: 621, SEQ ID NO: 622, SEQ ID NO: 623, SEQ ID NO: 624, SEQ ID NO: 625, SEQ ID NO: 626, SEQ ID NO: 627, SEQ ID NO: 628, SEQ ID NO: 629, SEQ ID NO: 630, SEQ ID NO: 631, SEQ ID NO: 632, SEQ ID NO: 633, SEQ ID NO: 634, SEQ ID NO: 635, SEQ ID NO: 636, SEQ ID NO: 637, SEQ ID NO: 638, SEQ ID NO: 639, SEQ ID NO: 640, SEQ ID NO: 641, SEQ ID NO: 642, SEQ ID NO: 643, SEQ ID NO: 644, SEQ ID NO: 645, SEQ ID NO: 646, SEQ ID NO: 647, SEQ ID NO: 648, SEQ ID NO: 649, SEQ ID NO: 650, SEQ ID NO: 651, SEQ ID NO: 652, SEQ ID NO: 653, SEQ ID NO: 654, SEQ ID NO: 655, SEQ ID NO: 656, SEQ ID NO: 657, SEQ ID NO: 658, SEQ ID NO: 659, SEQ ID NO: 660, SEQ ID NO: 661, SEQ ID NO: 662, SEQ ID NO: 663, SEQ ID NO: 664, SEQ ID NO: 665, SEQ ID NO: 666, SEQ ID NO: 667, SEQ ID NO: 668, SEQ ID NO: 669, SEQ ID NO: 670, SEQ ID NO: 671, SEQ ID NO: 672, SEQ ID NO: 673, SEQ ID NO: 674, SEQ ID NO: 675, SEQ ID NO: 676, SEQ ID NO: 677, SEQ ID NO: 678, SEQ ID NO: 679, SEQ ID NO: 680, SEQ ID NO: 681, SEQ ID NO: 682, SEQ ID NO: 683, SEQ ID NO: 684, SEQ ID NO: 685, SEQ ID NO: 686, SEQ ID NO: 687, SEQ ID NO: 688, SEQ ID NO: 689, SEQ ID NO: 690, SEQ ID NO: 691, SEQ ID NO: 692, SEQ ID NO: 693, SEQ ID NO: 694, SEQ ID NO: 695, SEQ ID NO: 696, SEQ ID NO: 697, SEQ ID NO: 698, SEQ ID NO: 699, SEQ ID NO: 700, SEQ ID NO: 701, SEQ ID NO: 702, SEQ ID NO: 703, SEQ ID NO: 704, SEQ ID NO: 705, SEQ ID NO: 706, SEQ ID NO: 707, SEQ ID NO: 708, SEQ ID NO: 709, SEQ ID NO: 710, SEQ ID NO: 711, SEQ ID NO: 712, SEQ ID NO: 713, SEQ ID NO: 714, SEQ ID NO: 715, SEQ ID NO: 716, SEQ ID NO: 717, SEQ ID NO: 718, SEQ ID NO: 719, SEQ ID NO: 720, SEQ ID NO: 721, SEQ ID NO: 722, SEQ ID NO: 723, SEQ ID NO: 724, SEQ ID NO: 725, SEQ ID NO: 726, SEQ ID NO: 727, SEQ ID NO: 728, SEQ ID NO: 729, SEQ ID NO: 730, SEQ ID NO: 731, SEQ ID NO: 732, SEQ ID NO: 733, SEQ ID NO: 734, SEQ ID NO: 735, SEQ ID NO: 736, SEQ ID NO: 737, SEQ ID NO: 738, SEQ ID NO: 739, SEQ ID NO: 740, SEQ ID NO: 741, SEQ ID NO: 742, SEQ ID NO: 743, SEQ ID NO: 744, SEQ ID NO: 745, SEQ ID NO: 746, SEQ ID NO: 747, SEQ ID NO: 748, SEQ ID NO: 749, SEQ ID NO: 750, SEQ ID NO: 751, SEQ ID NO: 752, SEQ ID NO: 753, SEQ ID NO: 754, SEQ ID NO: 755, SEQ ID NO: 756, SEQ ID NO: 757, SEQ ID NO: 758, SEQ ID NO: 759, SEQ ID NO: 760, SEQ ID NO: 761, SEQ ID NO: 762, SEQ ID NO: 763, SEQ ID NO: 764, SEQ ID NO: 765, SEQ ID NO: 766, SEQ ID NO: 767, SEQ ID NO: 768, SEQ ID NO: 769, SEQ ID NO: 770, SEQ ID NO: 771, SEQ ID NO: 772, SEQ ID NO: 773, SEQ ID NO: 774, SEQ ID NO: 775, SEQ ID NO: 776, SEQ ID NO: 777, SEQ ID NO: 778, SEQ ID NO: 779, SEQ ID NO: 780, SEQ ID NO: 781, SEQ ID NO: 782, SEQ ID NO: 783, SEQ ID NO: 784, SEQ ID NO: 785, SEQ ID NO: 786, SEQ ID NO: 787, SEQ ID NO: 788, SEQ ID NO: 789, SEQ ID NO: 790, SEQ ID NO: 791, SEQ ID NO: 792, SEQ ID NO: 793, SEQ ID NO: 794, SEQ ID NO: 795, SEQ ID NO: 796, SEQ ID NO: 797, SEQ ID NO: 798, SEQ ID NO: 799, SEQ ID NO: 800, SEQ ID NO: 801, SEQ ID NO: 802, SEQ ID NO: 803, SEQ ID NO: 804, SEQ ID NO: 805, SEQ ID NO: 806, SEQ ID NO: 807, SEQ ID NO: 808, SEQ ID NO: 809, SEQ ID NO: 810, SEQ ID NO: 811, SEQ ID NO: 812, SEQ ID NO: 813, SEQ ID NO: 814, SEQ ID NO: 815, SEQ ID NO: 816, SEQ ID NO: 817, SEQ ID NO: 818, SEQ ID NO: 819, SEQ ID NO: 820, SEQ ID NO: 821, SEQ ID NO: 822, SEQ ID NO: 823, SEQ ID NO: 824, SEQ ID NO: 825, SEQ ID NO: 826, SEQ ID NO: 827, SEQ ID NO: 828, SEQ ID NO: 829, SEQ ID NO: 830, SEQ ID NO: 831, SEQ ID NO: 832, SEQ ID NO: 833, SEQ ID NO: 834, SEQ ID NO: 835, SEQ ID NO: 836, SEQ ID NO: 837, SEQ ID NO: 838, SEQ ID NO: 839, SEQ ID NO: 840, SEQ ID NO: 841, SEQ ID NO: 842, SEQ ID NO: 843, SEQ ID NO: 844, SEQ ID NO: 845, SEQ ID NO: 846, SEQ ID NO: 847, SEQ ID NO: 848, SEQ ID NO: 849, SEQ ID NO: 850, SEQ ID NO: 851, SEQ ID NO: 852, SEQ ID NO: 853, SEQ ID NO: 854, SEQ ID NO: 855, SEQ ID NO: 856, SEQ ID NO: 857, SEQ ID NO: 858, SEQ ID NO: 859, SEQ ID NO: 860, SEQ ID NO: 861, SEQ ID NO: 862, SEQ ID NO: 863, SEQ ID NO: 864, SEQ ID NO: 865, SEQ ID NO: 866, SEQ ID NO: 867, SEQ ID NO: 868, SEQ ID NO: 869, SEQ ID NO: 870, SEQ ID NO: 871, SEQ ID NO: 872, SEQ ID NO: 873, SEQ ID NO: 874, SEQ ID NO: 875, SEQ ID NO: 876, SEQ ID NO: 877, SEQ ID NO: 878, SEQ ID NO: 879, SEQ ID NO: 880, SEQ ID NO: 881, SEQ ID NO: 882, SEQ ID NO: 883, SEQ ID NO: 884, SEQ ID NO: 885, SEQ ID NO: 886, SEQ ID NO: 887, SEQ ID NO: 888, SEQ ID NO: 889, SEQ ID NO: 890, SEQ ID NO: 891, SEQ ID NO: 892, SEQ ID NO: 893, SEQ ID NO: 894, SEQ ID NO: 895, SEQ ID NO: 896, SEQ ID NO: 897, SEQ ID NO: 898, SEQ ID NO: 899, SEQ ID NO: 900, SEQ ID NO: 901, SEQ ID NO: 902, SEQ ID NO: 903, SEQ ID NO: 904, SEQ ID NO: 905, SEQ ID NO: 906, SEQ ID NO: 907, SEQ ID NO: 908, SEQ ID NO: 909, SEQ ID NO: 910, SEQ ID NO: 911, SEQ ID NO: 912, SEQ ID NO: 913, SEQ ID NO: 914, SEQ ID NO: 915, SEQ ID NO: 916, SEQ ID NO: 917, SEQ ID NO: 918, SEQ ID NO: 919, SEQ ID NO: 920, SEQ ID NO: 921, SEQ ID NO: 922, SEQ ID NO: 923, SEQ ID NO: 924, SEQ ID NO: 925, SEQ ID NO: 926, or SEQ ID NO: 927, or a variant thereof containing one, two or three amino acid substitutions, or one amino acid deletion.

In an eleventh aspect the present invention relates to a monomeric peptide consisting of a sequence of amino acids as defined in any one of SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, SEQ ID NO: 376, SEQ ID NO: 377, SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451, SEQ ID NO: 452, SEQ ID NO: 453, SEQ ID NO: 454, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, SEQ ID NO: 524, SEQ ID NO: 525, SEQ ID NO: 526, SEQ ID NO: 527, SEQ ID NO: 528, SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, SEQ ID NO: 588, SEQ ID NO: 589, SEQ ID NO: 590, SEQ ID NO: 591, SEQ ID NO: 592, SEQ ID NO: 593, SEQ ID NO: 594, SEQ ID NO: 595, SEQ ID NO: 596, SEQ ID NO: 597, SEQ ID NO: 598, SEQ ID NO: 599, SEQ ID NO: 600, SEQ ID NO: 601, SEQ ID NO: 602, SEQ ID NO: 603, SEQ ID NO: 604, SEQ ID NO: 605, SEQ ID NO: 606, SEQ ID NO: 607, SEQ ID NO: 608, SEQ ID NO: 609, SEQ ID NO: 610, SEQ ID NO: 611, SEQ ID NO: 612, SEQ ID NO: 613, SEQ ID NO: 614, SEQ ID NO: 615, SEQ ID NO: 616, SEQ ID NO: 617, SEQ ID NO: 618, SEQ ID NO: 619, SEQ ID NO: 620, SEQ ID NO: 621, SEQ ID NO: 622, SEQ ID NO: 623, SEQ ID NO: 624, SEQ ID NO: 625, SEQ ID NO: 626, SEQ ID NO: 627, SEQ ID NO: 628, SEQ ID NO: 629, SEQ ID NO: 630, SEQ ID NO: 631, SEQ ID NO: 632, SEQ ID NO: 633, SEQ ID NO: 634, SEQ ID NO: 635, SEQ ID NO: 636, SEQ ID NO: 637, SEQ ID NO: 638, SEQ ID NO: 639, SEQ ID NO: 640, SEQ ID NO: 641, SEQ ID NO: 642, SEQ ID NO: 643, SEQ ID NO: 644, SEQ ID NO: 645, SEQ ID NO: 646, SEQ ID NO: 647, SEQ ID NO: 648, SEQ ID NO: 649, SEQ ID NO: 650, SEQ ID NO: 651, SEQ ID NO: 652, SEQ ID NO: 653, SEQ ID NO: 654, SEQ ID NO: 655, SEQ ID NO: 656, SEQ ID NO: 657, SEQ ID NO: 658, SEQ ID NO: 659, SEQ ID NO: 660, SEQ ID NO: 661, SEQ ID NO: 662, SEQ ID NO: 663, SEQ ID NO: 664, SEQ ID NO: 665, SEQ ID NO: 666, SEQ ID NO: 667, SEQ ID NO: 668, SEQ ID NO: 669, SEQ ID NO: 670, SEQ ID NO: 671, SEQ ID NO: 672, SEQ ID NO: 673, SEQ ID NO: 674, SEQ ID NO: 675, SEQ ID NO: 676, SEQ ID NO: 677, SEQ ID NO: 678, SEQ ID NO: 679, SEQ ID NO: 680, SEQ ID NO: 681, SEQ ID NO: 682, SEQ ID NO: 683, SEQ ID NO: 684, SEQ ID NO: 685, SEQ ID NO: 686, SEQ ID NO: 687, SEQ ID NO: 688, SEQ ID NO: 689, SEQ ID NO: 690, SEQ ID NO: 691, SEQ ID NO: 692, SEQ ID NO: 693, SEQ ID NO: 694, SEQ ID NO: 695, SEQ ID NO: 696, SEQ ID NO: 697, SEQ ID NO: 698, SEQ ID NO: 699, SEQ ID NO: 700, SEQ ID NO: 701, SEQ ID NO: 702, SEQ ID NO: 703, SEQ ID NO: 704, SEQ ID NO: 705, SEQ ID NO: 706, SEQ ID NO: 707, SEQ ID NO: 708, SEQ ID NO: 709, SEQ ID NO: 710, SEQ ID NO: 711, SEQ ID NO: 712, SEQ ID NO: 713, SEQ ID NO: 714, SEQ ID NO: 715, SEQ ID NO: 716, SEQ ID NO: 717, SEQ ID NO: 718, SEQ ID NO: 719, SEQ ID NO: 720, SEQ ID NO: 721, SEQ ID NO: 722, SEQ ID NO: 723, SEQ ID NO: 724, SEQ ID NO: 725, SEQ ID NO: 726, SEQ ID NO: 727, SEQ ID NO: 728, SEQ ID NO: 729, SEQ ID NO: 730, SEQ ID NO: 731, SEQ ID NO: 732, SEQ ID NO: 733, SEQ ID NO: 734, SEQ ID NO: 735, SEQ ID NO: 736, SEQ ID NO: 737, SEQ ID NO: 738, SEQ ID NO: 739, SEQ ID NO: 740, SEQ ID NO: 741, SEQ ID NO: 742, SEQ ID NO: 743, SEQ ID NO: 744, SEQ ID NO: 745, SEQ ID NO: 746, SEQ ID NO: 747, SEQ ID NO: 748, SEQ ID NO: 749, SEQ ID NO: 750, SEQ ID NO: 751, SEQ ID NO: 752, SEQ ID NO: 753, SEQ ID NO: 754, SEQ ID NO: 755, SEQ ID NO: 756, SEQ ID NO: 757, SEQ ID NO: 758, SEQ ID NO: 759, SEQ ID NO: 760, SEQ ID NO: 761, SEQ ID NO: 762, SEQ ID NO: 763, SEQ ID NO: 764, SEQ ID NO: 765, SEQ ID NO: 766, SEQ ID NO: 767, SEQ ID NO: 768, SEQ ID NO: 769, SEQ ID NO: 770, SEQ ID NO: 771, SEQ ID NO: 772, SEQ ID NO: 773, SEQ ID NO: 774, SEQ ID NO: 775, SEQ ID NO: 776, SEQ ID NO: 777, SEQ ID NO: 778, SEQ ID NO: 779, SEQ ID NO: 780, SEQ ID NO: 781, SEQ ID NO: 782, SEQ ID NO: 783, SEQ ID NO: 784, SEQ ID NO: 785, SEQ ID NO: 786, SEQ ID NO: 787, SEQ ID NO: 788, SEQ ID NO: 789, SEQ ID NO: 790, SEQ ID NO: 791, SEQ ID NO: 792, SEQ ID NO: 793, SEQ ID NO: 794, SEQ ID NO: 795, SEQ ID NO: 796, SEQ ID NO: 797, SEQ ID NO: 798, SEQ ID NO: 799, SEQ ID NO: 800, SEQ ID NO: 801, SEQ ID NO: 802, SEQ ID NO: 803, SEQ ID NO: 804, SEQ ID NO: 805, SEQ ID NO: 806, SEQ ID NO: 807, SEQ ID NO: 808, SEQ ID NO: 809, SEQ ID NO: 810, SEQ ID NO: 811, SEQ ID NO: 812, SEQ ID NO: 813, SEQ ID NO: 814, SEQ ID NO: 815, SEQ ID NO: 816, SEQ ID NO: 817, SEQ ID NO: 818, SEQ ID NO: 819, SEQ ID NO: 820, SEQ ID NO: 821, SEQ ID NO: 822, SEQ ID NO: 823, SEQ ID NO: 824, SEQ ID NO: 825, SEQ ID NO: 826, SEQ ID NO: 827, SEQ ID NO: 828, SEQ ID NO: 829, SEQ ID NO: 830, SEQ ID NO: 831, SEQ ID NO: 832, SEQ ID NO: 833, SEQ ID NO: 834, SEQ ID NO: 835, SEQ ID NO: 836, SEQ ID NO: 837, SEQ ID NO: 838, SEQ ID NO: 839, SEQ ID NO: 840, SEQ ID NO: 841, SEQ ID NO: 842, SEQ ID NO: 843, SEQ ID NO: 844, SEQ ID NO: 845, SEQ ID NO: 846, SEQ ID NO: 847, SEQ ID NO: 848, SEQ ID NO: 849, SEQ ID NO: 850, SEQ ID NO: 851, SEQ ID NO: 852, SEQ ID NO: 853, SEQ ID NO: 854, SEQ ID NO: 855, SEQ ID NO: 856, SEQ ID NO: 857, SEQ ID NO: 858, SEQ ID NO: 859, SEQ ID NO: 860, SEQ ID NO: 861, SEQ ID NO: 862, SEQ ID NO: 863, SEQ ID NO: 864, SEQ ID NO: 865, SEQ ID NO: 866, SEQ ID NO: 867, SEQ ID NO: 868, SEQ ID NO: 869, SEQ ID NO: 870, SEQ ID NO: 871, SEQ ID NO: 872, SEQ ID NO: 873, SEQ ID NO: 874, SEQ ID NO: 875, SEQ ID NO: 876, SEQ ID NO: 877, SEQ ID NO: 878, SEQ ID NO: 879, SEQ ID NO: 880, SEQ ID NO: 881, SEQ ID NO: 882, SEQ ID NO: 883, SEQ ID NO: 884, SEQ ID NO: 885, SEQ ID NO: 886, SEQ ID NO: 887, SEQ ID NO: 888, SEQ ID NO: 889, SEQ ID NO: 890, SEQ ID NO: 891, SEQ ID NO: 892, SEQ ID NO: 893, SEQ ID NO: 894, SEQ ID NO: 895, SEQ ID NO: 896, SEQ ID NO: 897, SEQ ID NO: 898, SEQ ID NO: 899, SEQ ID NO: 900, SEQ ID NO: 901, SEQ ID NO: 902, SEQ ID NO: 903, SEQ ID NO: 904, SEQ ID NO: 905, SEQ ID NO: 906, SEQ ID NO: 907, SEQ ID NO: 908, SEQ ID NO: 909, SEQ ID NO: 910, SEQ ID NO: 911, SEQ ID NO: 912, SEQ ID NO: 913, SEQ ID NO: 914, SEQ ID NO: 915, SEQ ID NO: 916, SEQ ID NO: 917, SEQ ID NO: 918, SEQ ID NO: 919, SEQ ID NO: 920, SEQ ID NO: 921, SEQ ID NO: 922, SEQ ID NO: 923, SEQ ID NO: 924, SEQ ID NO: 925, SEQ ID NO: 926, or SEQ ID NO: 927, or a variant thereof containing one or two amino acid substitutions, or one amino acid deletion.

In a twelfth aspect the present invention relates to a monomeric polypeptide from 10 to 80 amino acids in length, which polypeptide comprises or consist of two, three or four consecutive sequences of amino acids as independently defined herein, which two, three or four consecutive sequences of amino acids is optionally separated by, or having in the N- or C-terminal of the polypeptide, a sequence of amino acids selected from G, R, GG, GR, RG, RR, RRR, GGG, GGR, RGG, GRG, RRG, GRR, or RGR. In some specific embodiments, a polypeptide may consist of the sequence of amino acids selected from RRFYPRGQGVFLEGATNTASWFRSRGFQFPRGQGIG (SEQ ID NO:950), RNGVKGFNCYFCLQSYGPTYGVGYQPNNLDSKVGGNYLYCRLFRYKGTQGR (SEQ ID NO:951), QTQTNGSQSIIAGCGNLTTRTQKRFANGATWC (SEQ ID NO:952), DCEGKYHKNNKSWCEAVHRSYITPG (SEQ ID NO:953), TVRDPQTCDITESNKKFIPLGCGQLTPTWGRR (SEQ ID NO:954), RGPCNGVEGRGTPCNGVGRGGVEGFN (SEQ ID NO:934), RGKVGGNYGRGDSKVGGRG (SEQ ID NO:935), RGTNGVGYGRGFQPTNGGRGGVGYQP (SEQ ID NO:936), RGCYGVSPGRGFKCYGVGRGVSPTKL (SEQ ID NO:937), RGPLSETKGRGKCTLKSGRGSETKCT (SEQ ID NO:938), RGTVCGPKGRGPKKSTNGRGVKNKCV (SEQ ID NO:939), RGKVGGNYQNRLDSKVGGRN (SEQ ID NO:940), RRGPCNGVENRTPSNGVENRNGVEGFNNRSTPSNG (SEQ ID NO:941), RRRGSTPCNGVEGFQSNGVEGFNCWQRR (SEQ ID NO:942), RGTNGVGYNNRFQPTNGRNRGVGYQPRN (SEQ ID NO:943), RGASTEKSNRNGINITRQRRLLHAPATVG (SEQ ID NO:944), RRGFKSYGVSPTKLNDSKVGGNYQNRLDSKVGGNY (SEQ ID NO:945), RRSTPSNGVERRGVEGFNENRFQPTNGRNRGVGYQP (SEQ ID NO:946), RRGASTEKSNRNGINITRQLLHAPATVRTNGVGYG (SEQ ID NO:947), and RRKSTNLVGGATVTGPGGGVKNKSVGGPLSETK (SEQ ID NO:948), and RRKSTNLVGGQLTPTWGGGVKNKSVGGPLSETK (SEQ ID NO:949), or similar constructs. A similar construct to any such polypeptide includes, without limitation, a variant comprising one, two, three, or four amino acid substitutions, insertions or deletions, which variant has no more than one amino acid substitution, insertion or deletion per three, such as per four, such as per five, such as per six consecutive amino acids of the amino acid sequence of said SEQ ID NO.

In a thirteenth aspect the present invention relates to a multimeric peptide, such as a dimeric peptide, comprising at least a first monomeric peptide or polypeptide as defined herein, covalently joined to at least a second monomeric peptide or polypeptide independently as defined herein, the monomeric polypeptides being covalently joined, such as joined by a disulfide (S—S) bond between a Cys residue in each monomeric peptide.

In a fourteenth aspect, the present invention relates to a conjugate or fusion protein comprising a monomeric peptide as defined herein, a monomeric polypeptide as defined herein, or a multimeric polypeptide as defined herein, and a second moiety, such as a polymer or carrier molecule.

In a fifteenth aspect, the present invention relates to a combination comprising: (a) a first monomeric peptide as defined herein and a second monomeric peptide as defined herein, or (b) a first polypeptide as defined herein and a second polypeptide as defined herein, or (c) the polypeptides of SEQ ID NOS:951, 951, 952, 953 and 954, or (d) the polypeptides of SEQ ID NOS:945, 946, 947, 948 and 950.

In a sixteenth aspect, the present invention relates to a nucleic acid encoding a monomeric peptide as defined herein, a monomeric polypeptide as defined herein, a multimeric peptide as defined herein, or a combination of monomeric peptides, monomeric polypeptides, or multimeric peptides as defined herein.

In a seventeenth aspect, the present invention relates to a vector comprising a nucleic acid as defined in the preceding aspect.

In an eighteenth aspect the present invention relates to a pharmaceutical composition comprising a monomeric peptide or polypeptide as defined herein, or a multimeric, such as dimeric, peptide as defined herein, or a combination according to the invention, or a nucleic acid as defined herein, or a vector as defined herein, optionally further comprising a pharmaceutically acceptable diluent or vehicle and optionally an immunological adjuvant, such as IMM-101.

In a nineteenth aspect the present invention relates to a pharmaceutical composition comprising a monomeric peptide or polypeptide as defined herein, or a multimeric, such as dimeric, peptide as defined herein, or a combination according to the invention, formulated in a peptide slow-release formulation. In some embodiments this peptide slow-release formulation comprises a low viscosity, non-liquid crystalline, mixture of:

a) 25-55 wt. % of at least one diacyl glycerol and/or at least one tocopherol;
b) 25-55 wt. % of at least one phospholipid component comprising phospholipids having
i) polar head groups comprising more than 50% phosphatidyl ethanolamine, and
ii) two acyl chains each independently having 16 to 20 carbons wherein at least one acyl chain has at least one unsaturation in the carbon chain, and there are no more than four unsaturations over two carbon chains;
   c) 5-25 wt. % of at least one biocompatible, oxygen containing, low viscosity organic solvent;
   wherein 0.1-10 wt. % of at least one monomeric peptide, polypeptide or multimeric peptide is dissolved or dispersed in the low viscosity mixture; and wherein the pre-formulation forms, or is capable of forming, at least one non-lamellar liquid crystalline phase structure upon contact with an aqueous fluid.

In a twentieth aspect the present invention relates to a method for reducing and/or delaying pathological effects of an infection with corona virus, such as with human Wuhan seafood market pneumonia virus isolate Wuhan-Hu-1, in a human infected with such virus, the method comprising administering an effective amount of a monomeric peptide or polypeptide as defined herein, or multimeric, such as dimeric, peptide as defined herein, or combination as defined herein, or a nucleic acid as defined herein, or a vector as defined herein, or a pharmaceutical composition as defined herein.

In a twenty-first aspect the present invention relates to a method for inducing a therapeutic or ameliorating immune response against corona virus, such as with human Wuhan seafood market pneumonia virus isolate Wuhan-Hu-1, the method comprising administering an effective amount of a monomeric peptide or polypeptide as defined herein, or dimeric, peptide as defined herein, or a combination as defined herein, or a nucleic acid as defined herein, or a vector as defined herein, or a pharmaceutical composition as defined herein.

In a twenty-second aspect, the present invention relates to a method of inducing immunity in an animal, comprising administering at least once an immunogenically effective amount of a monomeric peptide or polypeptide as defined herein, or multimeric peptide as defined herein, or combination as defined herein, or nucleic acid as defined herein, or vector as defined herein, or a pharmaceutical composition as defined herein, so as to induce immunity against corona virus, such as against human Wuhan seafood market pneumonia virus isolate Wuhan-Hu-1, in the animal.

In a twenty-third aspect the present invention relates to the use of a monomeric peptide or polypeptide as defined herein, or multimeric, such as dimeric, peptide as defined herein, or combination as defined herein, or a nucleic acid as defined herein, or a vector as defined herein, for diagnostic use.

In a twenty-fourth aspect the present invention relates to the use of monomeric peptide or polypeptide as defined herein, or multimeric, such as dimeric, peptide as defined herein, or combination as defined herein, or nucleic acid as defined herein, or vector as defined herein; in the characterization of corona virus, such as with human Wuhan seafood market pneumonia virus isolate Wuhan-Hu-1 in vitro.

### Definitions

The term “Wuhan seafood market pneumonia virus isolate Wuhan-Hu-1” also referred to as “Covid-19 virus” or “SARS-CoV-2” refers to the virus encoded by the sequence identified as NCBI Reference Sequence: NC_045512.2 and mutants thereof, including naturally occurring or synthetic mutants thereof. Naturally occurring mutants of Covid-19 virus include, but are not limited to, those comprising an E to K substitution of residue 484 in SEQ ID NO:1 and/or an N to Y substitution in residue 501 of SEQ ID NO:1, i.e., the spike protein (see, e.g., GenBank: QQQ47833.1 and Tegally H., et al. “Emergence and rapid spread of a new severe acute respiratory syndrome-related coronavirus 2 (SARS-CoV-2) lineage with multiple spike mutations in South Africa”, medRxiv preprint doi https address doi.org 10.1101/2020.12.21.20248640, posted Dec. 22, 2020).

When terms such as “one”, “a” or “an” are used in this disclosure they mean “at least one”, or “one or more” unless otherwise indicated. Further, the term “comprising” is intended to mean “including” and thus allows for the presence of other constituents, features, conditions, or steps than those explicitly recited.

As used herein a “monomeric peptide” refers to a linear peptide, optionally a linear peptide segment which is part of a monomeric polypeptide as described herein. A monomeric peptide or peptide segment may, for example, comprise between 5 and 20 amino acids, such as between 5 and 15 amino acids, such as between 5 and 12 amino acids. For example, a monomeric peptide can be at least 5, 6, 7, 8, 9, or 10 amino acids in length, such as 6 or 7 amino acids in length. A monomeric peptide can also or alternatively be no more than 12, 11, 10, 9, 8, 7, 6, or 5 amino acids in length.

As used herein a “monomeric polypeptide” refers to a linear peptide sequence or subunit, optionally comprising one or more intrachain bonds in which two different, non-adjacent amino acids are interconnected, e.g., via a Cys-Cys bond formed between two different cysteine residues, also known as disulphide bond, or an intrachain bond between two residues independently selected from Lys, Ser, Cys, Asp and Glu, such as a thioether bond or an oxime bond or through a PEG linker. Typically, a monomeric polypeptide comprises one, two, three, four, five or more monomeric peptides according to any aspects or aspects described herein. In some embodiments, the amino acid sequences of two or more monomeric peptides as defined herein may overlap at least partially in the monomeric polypeptide sequence. Preferably, a monomeric polypeptide has a length in the range of 10 and 80 amino acids. Other specific lengths and ranges contemplated for a monomeric polypeptide as defined herein are described in the section entitled “Specific embodiments of the invention.”

As used herein a “multimeric peptide” or “oligomeric peptide” or “multimeric polypeptide” refers to an assembly of two or more different or identical linear peptide sequences or subunits, preferably interconnected or assembled by one or more chemical bonds or a linker. Preferably the peptide sequences are interconnected by one or more, such as one covalent bond, such as an intermolecular disulfide (S—S) bond between two Cys residues, a methylated peptide bond between a N-s-methylated Lys side-chain and the side-chain of an Asp or Glu residue, an oxime bond, a thioether bond, or a non-covalent bond, such as in a n-stacking of rings wherein a residue in a first peptide repeat is linked to a residue in a second peptide repeat. Preferably the multimeric peptide is a dimeric peptide. The term includes a dimeric (or dimer) peptide suitably formed by a chemical linking of two linear peptide sequences. The term “multimeric peptide” further includes an assembly of 2, 3, 4, 5, 6, 7, 8, 9 or 10 different or identical peptide sequences. In some embodiments, the multimeric peptide is a dimeric peptide.

As used herein a “linker” refers to any compound suitable for assembly of the two or more different or identical linear peptide sequences or subunits into a multimeric peptide, or to any other therapeutically active compound, such as an immunomodulating compound. The term includes any linker found useful in peptide chemistry. Since the multimeric peptide may be assembled or connected by standard peptide bonds in a linear way, the term linker also includes a “peptide spacer”, also referred to as a “spacer”.

In some embodiments, the linker is not a peptide sequence. In some embodiments, the linker is not a branched peptide sequence.

In some embodiments, the linker does not itself contain a peptide sequence derived from or identical to a natural antigen.

In some embodiments, the linker has a molecular weight of less than 10 kDa, such as less than 9 kDa, such as less than 8 kDa, such as less than 7 kDa, such as less than 6 kDa, such as less than 5 kDa, such as less than 4 kDa, such as less than 3 kDa, such as less than 2 kDa, such as less than 1.5 kDa, such as less than 1 kDa, such as less than 0.5 kDa, such as less than 0.2 kDa. In some embodiments, wherein the multimeric peptide is a dimeric peptide, the linker is not linking the two peptide sequences from one terminal cysteine in the first peptide to a second terminal cysteine in the second peptide.

In some embodiments, the linker is not linking the two or more peptide sequences through a terminal cysteine in any one of the peptides.

In some embodiments, the linker is not linking from a cysteine residue.

The term “cell-penetrating peptide” as used herein refers to any peptide with the capability to translocate across the plasma membrane into either cytoplasmic and/or nuclear compartments of eukaryotic and/or prokaryotic cells, such as into cytoplasm, nucleus, lysosome, endoplasmatic reticulum, golgi apparatus, mitocondria and/or chloroplast, seemingly energy-independently. This capability to translocate across the plasma membrane of a “cell-penetrating peptide” according to the invention may be non-invasive, energy-independent, non-saturable, and/or receptor independent. In one embodiment the term “cell-penetrating peptide” refers to a peptide, which is demonstrated to translocate across a plasma membrane as determined by the assay in example 5. It is to be understood that a cell-penetrating peptide according to the present invention may be translocated across the membrane with the sequence complete and intact, or alternatively partly degraded, but in a form where the antigens contained within this peptide is able to be presented within the cell to stimulate an immune response. Accordingly, a cell-penetrating peptide according to the present invention is a peptide that may be demonstrated to translocate across a plasma membrane as determined by the assay in example 5 and be demonstrated to stimulate an effective immune response.

The monomeric peptide according to the present invention may be provided in any pharmaceutically acceptable salt, such as in a salt of acetat or HCl.

The amino acids used in the amino acid sequences according to the invention may be in both L- and/or D-form. It is to be understood that both L- and D-forms may be used for different amino acids within the same peptide sequence. In some embodiments the amino acids within the peptide sequence are in L-form, such as natural amino acids. It is to be understood that any known antigen may be used in the constructs according to the present invention.

In some specific embodiments, the first 1, 2, or 3 amino acids in the N-terminal of the amino acid sequences according to the invention are in the D-form. It is assumed that the N-terminal trimming and thereby degradation of the peptides are somewhat delayed by having amino acids of the D-form in the N-terminal of these peptides according to the present invention. Alternatively, and in some embodiments, the first 1, 2, or 3 amino acids in the N-terminal of the amino acid sequences according to the invention are amino acids in beta or gamma forms. Beta amino acids have their amino group bonded to the beta carbon rather than the alpha carbon as in the 20 standard natural amino acids.

Alternatively, the first 1, 2, or 3 amino acids in the N-terminal of the amino acid sequences according to the invention may be modified by incorporation of fluorine, or alternatively cyclic amino acids or other suitable non-natural amino acids are used.

A “variant” or “analogue” of a peptide refers to a peptide having an amino acid sequence that is substantially identical to a reference peptide, typically a native or “parent” polypeptide. The peptide variant may possess one or more amino acid substitutions, deletions, and/or insertions at certain positions within the native amino acid sequence.

“Conservative” amino acid substitutions are those in which an amino acid residue is replaced with an amino acid residue having a side chain with similar physicochemical properties. Families of amino acid residues having similar side chains are known in the art, and include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, norleucine, isoleucine, proline, phenylalanine, methionine), beta-branched side chains (e.g., threonine, valine, isoleucine) and aromatic side chains (e.g., tyrosine, phenylalanine, tryptophan, histidine). Norleucin may be referred to as Nle. A particular form of conservative amino acid substitutions includes those with amino acids, which are not among the normal 20 amino acids encoded by the genetic code. Since preferred embodiments of the present invention entail use of synthetic peptides, it is unproblematic to provide such “non-naturally occurring” amino acid residues in the peptides disclosed herein, and thereby it is possible to exchange the natural saturated carbon chains in the side chains of amino acid residues with shorter or longer saturated carbon chains—for instance, lysine may be substituted with an amino acid having an the side chain —(CH₂)ₙNH₃, where n is different from 4, and arginine may be substituted with an amino acid having the side chain —(CH₂)ₙNHC(═NH₂)NH₂, where n is different from 3, etc. Similarly, the acidic amino acids aspartic acid and glutamic acid may be substituted with amino acid residues having the side chains —(CH₂)ₙCOOH, where n>2.

An “isolated” molecule is a molecule that is the predominant species in the composition wherein it is found with respect to the class of molecules to which it belongs (i.e., it makes up at least about 50% of the type of molecule in the composition and typically will make up at least about 70%, at least about 80%, at least about 85%, at least about 90%, at least about 95%, or more of the species of molecule, e.g., peptide, in the composition). Commonly, a composition of a peptide molecule will exhibit 98%-99% homogeneity for peptide molecules in the context of all present peptide species in the composition or at least with respect to substantially active peptide species in the context of proposed use.

The term “sequence of amino acids” as used herein refers to the specific sequence of amino acids connected by standard peptide bonds in standard N- to C-terminal direction. The peptide may contain only peptide bonds. However, the term does not exclude that an amino acid within a sequence may be connected, such as through the side chains, with another amino acid at a distant location within the peptide sequence, e.g., via a disulphide bridge between two cysteine residues so as to form an intrachain loop. Preferably a sequence of amino acids refers to a linear sequence of amino acids only, optionally with an intrachain disulphide bridge.

In the context of the present invention, “treatment” or “treating” refers to preventing, alleviating, managing, curing or reducing one or more symptoms or clinically relevant manifestations of a disease or disorder in a subject, unless contradicted by context. For example, “treatment” of a subject or patient in whom no symptoms or clinically relevant manifestations of a disease or disorder have been identified is preventive or prophylactic therapy, whereas “treatment” of a patient in whom symptoms or clinically relevant manifestations of a disease or disorder have been identified generally does not constitute preventive or prophylactic therapy. Typically, the subject is an animal, e.g., a mammal, such as a human.

Preferably the monomeric peptides and polypeptides defined herein represent epitopes recognized by the human immune system and/or by the immune system of another animal for which treatment is intended. The terms “epitope”, “antigenic determinant” and “antigenic site” are used interchangeably herein and denotes the region in an antigen or immunogen which is recognized by antibodies (in the case of antibody binding epitopes, also known as “B-cell epitopes”) or by T-cell receptors when the epitope is complexed to a Major histocompatibility complex (MHC) molecule (in the case of T-cell receptor binding epitopes, i.e. “T-cell epitopes”).

The term “animal” is in the present context in general intended to denote an animal species (preferably mammalian), including, but not limited to, humans (Homo sapiens) and domestic animals such as dogs (Canis domesticus), cats, rabbits, camels and dromedaries etc. and not just one single animal. However, the term also denotes a population of such an animal species.

“B cell antigen” means any antigen that naturally is or could be engineered to be recognized by a B cell, and that triggers an immune response in a B cell (e.g., an antigen that is specifically recognized by a B cell receptor on a B cell).

The term “immunogenically effective amount” has its usual meaning in the art, i.e. an amount of an immunogen, which is capable of inducing an immune response, which significantly engages pathogenic agents, which share immunological features with the immunogen.

The term “vaccine” is used for a composition comprising an immunogen and which is capable of inducing an immune response which is either capable of reducing the risk of developing a pathological condition or capable of inducing a therapeutically effective immune response which may aid in the cure of (or at least alleviate the symptoms of) a pathological condition.

The term “pharmaceutically acceptable” has its usual meaning in the art, i.e. it is used for a substance that can be accepted as part of a medicament for human use when treating the disease in question and thus the term effectively excludes the use of highly toxic substances that would worsen rather than improve the treated subject's condition.

A “T helper lymphocyte epitope” (a TH epitope) is peptide, which binds an MHC Class II molecule and can be presented on the surface of an antigen presenting cell (APC) bound to the MHC Class II molecule. An “immunological carrier” is generally a substance of matter which includes one or many TH epitopes, and which increase the immune response against an antigen to which it is coupled by ensuring that T-helper lymphocytes are activated and proliferate. Examples of known immunological carriers are the tetanus and diphtheria toxoids and keyhole limpet hemocyanin (KLH).

The peptides according to the present invention may be a helper T lymphocyte (HTL) inducing peptide comprising HTL epitopes. An “HTL inducing peptide” is a HLA Class II binding peptide that is capable of inducing a HTL response. Also, the peptides according to the present invention may in other embodiments be CTL inducing peptides comprising CTL epitopes in addition to or as an alternative to being a HTL inducing peptide. A “CTL inducing peptide” is an HLA Class I binding peptide that is capable of inducing a CTL response.

In some embodiments the epitopes used in the scaffold according to the present invention are CTL epitopes. A “CTL inducing peptide” is an HLA Class I binding peptide that is capable of inducing a CTL response. In other embodiments the epitopes used in the scaffold design according to the present invention are HTL inducing peptides. An “HTL inducing peptide” is a HLA Class II binding peptide that is capable of inducing a HTL response.

In other alternative embodiments, tryptophan or tryptophan derivatives are used in the sequence of amino acids as defined herein. Any suitable tryptophan derivatives may be used. As used herein “tryptophan derivatives” means an unnatural modified tryptophan amino acid residue including those disclosed in U.S. Pat. No. 7,232,803, such as tri tert.-butyltryptophan, di-tert-butyl tryptophan, 7-benzyloxytryptophan, homotryptophan, 5′-aminoethyltryptophan (available as side chain Boc and N-alpha FMOC derivative from RSP Amino Acids Analogues Inc, Boston, Mass., USA), N-Acetylhomotryptophan (Toronto Research), 7-Benzyloxytryptophan (Toronto Research), Homotryptophan (Toronto Research), and tryptophan residues which have been substituted at the 1-, 2-, 5- and/or 7-position of the indole ring, positions 1- or 2- being preferred e.g. 5′ hydroxy tryptophan.

The term “amino acid derivative”, sometimes used in the context of a “derivative thereof” referring to a specific amino acid, means an amino acid compound, wherein one or more chemical groups has been modified, added or removed as compared to the amino acid to which the amino acid compound is a derivative of, while still having an amine group and a carboxylic acid group, as well as a side chain of an amino acid and still being able to form peptide bonds. In some embodiments an amino acid derivative is a standard amino acid that has only been modified in the side chain of the amino acid. In some embodiments an amino acid derivative is a non-natural amino acid such as Dpr. In some embodiments an amino acid is a modified moiety which is incorporated into the chemically synthesized peptide or polypeptide and that comprises an activatable group that is linkable, after activation, to another peptide, such as Dpr(Ser), Lys(Ser), or Ornithine(Ser).

The term “basic amino acid” as used herein refers to any amino acid including both natural and non-natural amino acids that has an isoelectric point above 6.3 (such as above 7.4) as measured according to Kice & Marvell “Modern Principles of organic Chemsitry” (Macmillan, 1974) or Matthews and van Holde “Biochemistry” Cummings Publishing Company, 1996. Included within this definition are Arginine, Lysine, Homoarginine (Har, or Hr), and Histidine as well as derivatives thereof. Suitable non-natural basic amino acids are e.g. as described in U.S. Pat. No. 6,858,396. Suitable positively charged amino acids includes non-natural alpha amino acids available from Bachem AG and includes alpha-amino-glycine, alpha,gamma-diaminobutyric acid, ornithine, alpha, beta-diaminoproprionic acid, alpha-difluoromethyl-ornithine, 4-amino-piperidine-4-carboxylic acid, 2,6-diamino-4-hexynoic acid, beta-(1-piperazinyl)-alanine, 4,5-dehydro-lysine, delta-hydroxy-lysine, omega-hydroxy-norarginine, homoarginine, omega-amino-arginine, omega-methyl-arginine, alpha-methyl-histidine, 2,5-diiodo-histidine, 1-methyl-histidine, 3-methyl-histidine, beta-(2-pyridyl)-alanine, beta-(3-pyridyl)-alanine, beta-(2-quinolyl)-alanine, 3-amino-tyrosine, 4-amino-phenylalanine, and spinacine. Furthermore, any mono or dicarboxylic amino acid is a suitable positively charged amino acid.

The term “neutral amino acid” as used herein refers to an amino acid that has an isoelectric point above between 4.8 and 6.3 as measured according to Kice & Marvell “Modern Principles of organic Chemsitry” (Macmillan, 1974). The term “acidic amino acid” as used herein refers to an amino acid that has an isoelectric point below 4.8 as measured according to Kice & Marvell “Modern Principles of organic Chemsitry” (Macmillan, 1974).

Unless otherwise indicated amino acids are abbreviated and mentioned by their standard nomenclature known to the person skilled in the art, such as with reference to “nomenclature and symbolism for amino acids and peptides” by the international union of pure and applied chemistry (IUPAC) (www.iupac.org).

The term “antibody response” refers to the production of antibodies (e.g., IgM, IgA, IgG) which bind to an antigen of interest, this response is measured for instance by assaying sera by antigen ELISA.

The term “adjuvant” as used herein refers to any compound which, when delivered together or simultaneously with an antigen, non-specifically enhances the immune response to that antigen. Exemplary adjuvants include but are not limited to oil in water and water in oil adjuvants, aluminum-based adjuvants (e.g., AIOH, AIPO4, etc), and Montanide ISA 720.

The terms “patient” and “subject” refer to a mammal that may be treated using the methods of the present invention.

As used herein, the term “immune response” refers to the reactivity of an organism's immune system in response to an antigen. In vertebrates, this may involve antibody production, induction of cell-mediated immunity, and/or complement activation (e.g., phenomena associated with the vertebrate immune system's prevention and resolution of infection by microorganisms). In preferred embodiments, the term immune response encompasses but is not limited to one or more of a “lymphocyte proliferative response,” a “cytokine response,” and an “antibody response.”

The term “net charge” as used herein with reference to a peptide sequence refers to the total electric charge of the peptide sequence represented by the sum of charges of each individual amino acid in the peptide sequence, wherein each basic amino acid are given a charge of +1, each acidic amino acid a charge of −1, and each neutral amino acid a charge of 0. Accordingly, the net charge will depend on the number and identities of charged amino acids.

The specific natural antigen used in the peptide constructs according to the present invention is a protein or peptide sequence derived from a B cell antigen of Covid-19 virus.

Accordingly, as used herein, a “disease antigen” refers to an antigen confirmed or suspected to be involved in a corona virus infection.

### Carriers, Adjuvants and Vehicles—Delivery—Treatment Methods

A peptide, polypeptide, conjugate, fusion protein, combination, nucleic acid, vector or pharmaceutical composition according to the present invention may be used as a pharmaceutical, e.g., a vaccine. For example, the peptide, polypeptide, combination, nucleic acid, vector or pharmaceutical composition may be used in the prophylaxis and amelioration of a disease caused by virus infections with Corona virus, in particular Wuhan seafood market pneumonia virus isolate Wuhan-Hu-1, including naturally occurring or synthetic mutants thereof.

A peptide, polypeptide, conjugate, fusion protein, combination, nucleic acid, vector or pharmaceutical composition as described herein can be used in a method of inducing immunity in an animal, comprising administering at least once an immunogenically effective amount of the peptide, polypeptide, combination, nucleic acid, vector or pharmaceutical composition according to the invention, so as to induce immunity against corona virus, such as against human Wuhan seafood market pneumonia virus isolate Wuhan-Hu-1, in the animal. In some embodiments, the animal is a mammal, such as a human or domestic animal, such as a human. In some embodiments, the immunity comprises humoral immunity.

A peptide, polypeptide, conjugate, fusion protein, combination, nucleic acid, vector, or pharmaceutical composition as described herein can also be used in a method for inducing a therapeutic or ameliorating immune response against corona virus, such as against human Wuhan seafood market pneumonia virus isolate Wuhan-Hu-1, the method comprising administering an immunogenically effective amount of the peptide, polypeptide, combination, nucleic acid, vector or pharmaceutical composition as described herein. In some embodiments, the immune response comprises a humoral immune response.

The peptides, polypeptides, e.g., isolated peptides or polypeptides, conjugates, fusion proteins, combinations, nucleic acids, or vectors according to the invention may be delivered by various means and within various compositions, herein referred to as “compositions”, “vaccine compositions” or “pharmaceutical compositions”. The peptides, polypeptides, conjugates, fusion proteins, combinations, nucleic acids, and vectors of the present invention and pharmaceutical and vaccine compositions of the invention are useful for administration to mammals, particularly humans, to treat and/or prevent virus infection. Vaccine compositions containing the peptides, polypeptides, combinations, nucleic acids, or vectors of the invention are administered to a patient infected with the virus in question or to an individual susceptible to, or otherwise at risk for, virus infection to elicit an immune response against the specific antigens and thus enhance the patient's own immune response capabilities.

Various art-recognized delivery systems may be used to deliver the peptides, polypeptides, conjugates, fusion proteins, combinations, nucleic acids, or vectors into appropriate cells. The peptides, polypeptides, conjugates, fusion proteins, combinations, nucleic acids, or vectors can be delivered in a pharmaceutically acceptable carrier or as colloidal suspensions, or as powders, with or without diluents. They can be “naked” or associated with delivery vehicles and delivered using delivery systems known in the art.

A “pharmaceutically acceptable carrier” or “pharmaceutically acceptable adjuvant” is any suitable excipient, diluent, carrier and/or adjuvant which, by themselves, do not induce the production of antibodies harmful to the individual receiving the composition nor do they elicit protection. Preferably, a pharmaceutically acceptable carrier or adjuvant enhances the immune response elicited by an antigen. Suitable carriers or adjuvant typically comprise one or more of the compounds included in the following non-exhaustive list: large slowly metabolized macromolecules such as proteins, polysaccharides, polylactic acids, polyglycolic acids, polymeric amino acids, amino acid copolymers and inactive virus particles; aluminium hydroxide, aluminium phosphate (see International Patent Application Publication No. WO93/24148), alum (KAI(SO4)2.12H2O), or one of these in combination with 3-O-deacylated monophosphoryl lipid A (see International Patent Application Publication No. WO93/19780); N-acetyl-muramyl-L-threonyl-D-isoglutamine (see U.S. Pat. No. 4,606,918), N-acetyl-normuramyl-L-alanyl-D-isoglutamine, N-acetylmuramyl-L-alanyl-D-isoglutamyl-L-alanine2-(1′,2′-dipalmitoyl-sn-glycero-3-hydroxyphosphoryloxy) ethylamine; RIBI (ImmunoChem Research Inc., Hamilton, Mont., USA) which contains monophosphoryl lipid A (i.e., a detoxified endotoxin), trehalose-6,6-dimycolate, and cell wall skeleton (MPL+TDM+CWS) in a 2% squalene/Tween 80 emulsion. Any of the three components MPL, TDM or CWS may also be used alone or combined 2 by 2; adjuvants such as Stimulon (Cambridge Bioscience, Worcester, Mass., USA), SAF-1 (Syntex); adjuvants such as combinations between QS21 and 3-de-O-acetylated monophosphoryl lipid A (see International Application No. WO94/00153) which may be further supplemented with an oil-in-water emulsion (see, e.g., International Application Nos. WO95/17210, WO97/01640 and WO9856414) in which the oil-in-water emulsion comprises a metabolisable oil and a saponin, or a metabolisable oil, a saponin, and a sterol, or which may be further supplemented with a cytokine (see International Application No. WO98/57659); adjuvants such as MF-59 (Chiron), or poly[di(carboxylatophenoxy) phosphazene] based adjuvants (Virus Research Institute); blockcopolymer based adjuvants such as Optivax (Vaxcel, Cytrx) or inulin-based adjuvants, such as Algammulin and Gammalnulin (Anutech); Complete or Incomplete Freund's Adjuvant (CFA or IFA, respectively) or Gerbu preparations (Gerbu Biotechnik); a saponin such as QuilA, a purified saponin such as QS21, QS7 or QS17, -escin or digitonin; immunostimulatory oligonucleotides comprising unmethylated CpG dinucleotides such as [purine-purine-CG-pyrimidine-pyrimidine] oligonucleotides. These immunostimulatory oligonucleotides include CpG class A, B, and C molecules (Coley Pharmaceuticals), ISS (Dynavax), Immunomers (Hybridon). Immunostimulatory oligonucleotides may also be combined with cationic peptides as described, e.g., by Riedl et al. (2002); Immune Stimulating Complexes comprising saponins, for example Quil A (ISCOMS); excipients and diluents, which are inherently non-toxic and non-therapeutic, such as water, saline, glycerol, ethanol, isopropyl alcohol, DMSO, wetting or emulsifying agents, pH buffering substances, preservatives, and the like; a biodegradable and/or biocompatible oil such as squalane, squalene, eicosane, tetratetracontane, glycerol, peanut oil, vegetable oil, in a concentration of, e.g., 1 to 10% or 2.5 to 5%; vitamins such as vitamin C (ascorbic acid or its salts or esters), vitamin E (tocopherol), or vitamin A; carotenoids, or natural or synthetic flavanoids; trace elements, such as selenium; any Toll-like receptor ligand as reviewed in Barton and Medzhitov (2002).

For a further enhancement of the vaccine antigenic properties, could be to combine a well-known adjuvant with an oral immune modulant, such as IMID or adjuvant such as a Cox-2 inhibitor or a immunomodulating compound.

A further aspect of the invention is the use of the vaccine combined with adjuvant, and/or with an (oral) immunomodulating agent.

Other suitable adjuvants include response-selective C5a agonists, such as EP54 and EP67 described in Hung C Y et al. An agonist of human complement fragment C5a enhances vaccine immunity against Coccidioides infection. Vaccine (2012) and Kollessery G et al. Tumor-specific peptide-based vaccines containing the conformationally biased, response-selective C5a agonists EP54 and EP67 protect against aggressive large B cell lymphoma in a syngeneic murine model. Vaccine (2011) 29: 5904-10.

Other suitable adjuvants include an oil-in-water emulsion containing a stabilizing detergent, a micelle-forming agent and a biodegradable oil, such as Provax described in e.g. U.S. Pat. No. 5,585,103.

Any of the aforementioned adjuvants comprising 3-de-O-acetylated monophosphoryl lipid A, said 3-de-O-acetylated monophosphoryl lipid A may be forming a small particle (see International Application No. WO94/21292).

In any of the aforementioned adjuvants MPL or 3-de-O-acetylated monophosphoryl lipid A can be replaced by a synthetic analogue referred to as RC-529 or by any other amino-alkyl glucosaminide 4-phosphate (Johnson et al. 1999, Persing et al. 2002). Alternatively, it can be replaced by other lipid A analogues such as OM-197 (Byl et al. 2003).

Other suitable peptide vaccine adjuvants to be used in the pharmaceutical formulations according to the present invention includes adjuvants using heterogeneous Monophosphoryl Lipid A (MPL) derived from Salmonella minnesota R595, such as any synthetic analogs of MPL containing a single molecular species including synthetic Monophosphoryl Lipid A (MPLA (PHAD®), CAS Number 1246298-63-4), 3D-PHAD®, and 3D-(6A)-PHAD® from Avanti.

Other suitable peptide vaccine adjuvants to be used in the pharmaceutical formulations according to the present invention includes adjuvants from Invivogen including adjuvants containing synthetic immunostimulatory oligonucleotide (ODN) that contains unmethylated CpG dinucleotides, such as ODN 1585 VacciGrade™, ODN 1826 VacciGrade™, ODN 2006 VacciGrade™, and ODN 2395 VacciGrade™.

Other suitable peptide vaccine adjuvants to be used in the pharmaceutical formulations according to the present invention includes adjuvants from Immodulon Therapeutics Ltd, such as IMM-101, which is a heat-killed Mycobacterium obuense NCTC13365.

A “pharmaceutically acceptable vehicle” includes vehicles such as water, saline, physiological salt solutions, glycerol, ethanol, etc. Auxiliary substances such as wetting or emulsifying agents, pH buffering substances, preservatives may be included in such vehicles. Delivery systems known in the art are e.g. lipopeptides, peptide compositions encapsulated in poly-DL-lactide-co-glycolide (“PLG”), microspheres, peptide compositions contained in immune stimulating complexes (ISCOMS), multiple antigen peptide systems (MAPs), viral delivery vectors, particles of viral or synthetic origin, adjuvants, liposomes, lipids, microparticles or microcapsules, gold particles, nanoparticles, polymers, condensing agents, polysaccharides, polyamino acids, dendrimers, saponins, QS21, adsorption enhancing materials, fatty acids or, naked or particle absorbed cDNA.

The peptides, polypeptides, conjugates, fusion proteins, combinations, nucleic acids, or vectors may be delivered in oils such as Endocine™ and Montanide™ (Eurocine)—Montanide™ ISA 51 VG or Montanide™ ISA 720 VG (Seppic).

The adjuvant(s) may be stimulators of the innate immune system that can be given separately from the peptide such as Leukotriene B4 (LTB4) and granulocyte macrophage colony stimulating factor (GM-CSF), such as Sargramostim/Leukine (glycosylated GM-CSF) and Molgramostim (nonglycosylated GM-CSF).

Typically, a vaccine or vaccine composition is prepared as an injectable, either as a liquid solution or suspension. Injection may be subcutaneous, intramuscular, intravenous, intraperitoneal, intrathecal, intradermal, or intraepidermal. Other types of administration comprise electroporation, implantation, suppositories, oral ingestion, enteric application, inhalation, aerosolization or nasal spray or drops. Solid forms, suitable for dissolving in, or suspension in, liquid vehicles prior to injection may also be prepared. The preparation may also be emulsified or encapsulated in liposomes for enhancing adjuvant effect.

A liquid formulation may include oils, polymers, vitamins, carbohydrates, amino acids, salts, buffers, albumin, surfactants, or bulking agents. Preferably carbohydrates include sugar or sugar alcohols such as mono-, di-, tri-, oligo- or polysaccharides, or water-soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof. Sucrose is most preferred. “Sugar alcohol” is defined as a C4 to C8 hydrocarbon having an —OH group and includes galactitol, inositol, mannitol, xylitol, sorbitol, glycerol, and arabitol. Mannitol is most preferred. These sugars or sugar alcohols mentioned above may be used individually or in combination. There is no fixed limit to the amount used as long as the sugar or sugar alcohol is soluble in the aqueous preparation. Preferably, the sugar or sugar alcohol concentration is between 1.0% (w/v) and 7.0% (w/v), more preferable between 2.0 and 6.0% (w/v). Preferably amino acids include levorotary (L) forms of carnitine, arginine, and betaine; however, other amino acids may be added. Preferred polymers include polyvinylpyrrolidone (PVP) with an average molecular weight between 2,000 and 3,000, or polyethylene glycol (PEG) with an average molecular weight between 3,000 and 5,000. It is also preferred to use a buffer in the composition to minimize pH changes in the solution before lyophilization or after reconstitution. Any physiological buffer may be used, but citrate, phosphate, succinate, and glutamate buffers or mixtures thereof are preferred. Most preferred is a citrate buffer. Preferably, the concentration is from 0.01 to 0.3 molar. Surfactants that can be added to the formulation are shown in EP patent applications No. EP 0 270 799 and EP 0 268 110.

Additionally, the peptides or polypeptides according to the present invention may be chemically modified by covalent conjugation to a polymer to increase their circulating half-life, for example. Preferred polymers, and methods to attach them to peptides, are shown in U.S. Pat. Nos. 4,766,106; 4,179,337; 4,495,285; and 4,609,546. Preferred polymers are polyoxyethylated polyols and polyethylene glycol (PEG). PEG is soluble in water at room temperature and has the general formula:

R(O—CH2-CH2)nO—R,

where R can be hydrogen, or a protective group such as an alkyl or alkanol group. Preferably, the protective group has between 1 and 8 carbons, more preferably it is methyl. The symbol n is a positive integer, preferably between 1 and 1.000, more preferably between 2 and 500. The PEG has a preferred average molecular weight between 1000 and 40.000, more preferably between 2000 and 20.000, most preferably between 3.000 and 12.000. Preferably, PEG has at least one hydroxy group, more preferably it is a terminal hydroxy group. It is this hydroxy group which is preferably activated. However, it will be understood that the type and amount of the reactive groups may be varied to achieve a covalently conjugated PEG/polypeptide of the present invention.

Additionally, the peptides or polypeptides according to the present invention may be chemically modified by covalent conjugation to a fatty acid to increase their circulating half-life, for example. The fatty acid typically has the general formula: HOOC—R₁-R₂ wherein R₁ is a saturated or unsaturated alkyl chain of between 1 and 50 carbon atoms, preferably between 1 and 25 carbon atoms, such as 14 or 15 carbon atoms, which alkyl chain is optionally branched and optionally substituted, preferably with one or more halogen groups, hydroxyl groups, and/or amine groups; and wherein R₂ is hydrogen or —COOH.

Additionally, the peptides or polypeptides according to the present invention may be chemically modified by glycosylation with a carbohydrate on an amino acid. The carbohydrate has the general formula: Cₘ(H₂O)ₙ. The carbohydrate could be an amino glycoside, e.g., N-acetylgalactosamine (GalNac). The carbohydrate could be a mono- to penta-glycoside, preferably mono- to di-glycoside. The glycoside could be conjugated through the side chains of Lysine, Tryptophane, Serine, Threonine, Asparagine, Glutamine, Cysteine, or Arginine. Preferably Serine, Threonine, Asparagine, Glutamine; more preferably Asparagine or Glutamine.

Water soluble polyoxyethylated polyols are also useful in the present invention. They include polyoxyethylated sorbitol, polyoxyethylated glucose, polyoxyethylated glycerol (POG), etc. POG is preferred. One reason is because the glycerol backbone of polyoxyethylated glycerol is the same backbone occurring naturally in, for example, animals and humans in mono-, di-, triglycerides. Therefore, this branching would not necessarily be seen as a foreign agent in the body. The POG has a preferred molecular weight in the same range as PEG. The structure for POG is shown in Knauf et al., 1988, and a discussion of POG/IL-2 conjugates is found in U.S. Pat. No. 4,766,106.

Another drug delivery system for increasing circulatory half-life is the liposome. The peptides, polypeptides, conjugates, fusion proteins, and nucleic acids of the invention may also be administered via liposomes, which serve to target a particular tissue, such as lymphoid tissue, or to target selectively infected cells, as well as to increase the half-life of the peptide and nucleic acids composition. Liposomes include emulsions, foams, micelles, insoluble monolayers, liquid crystals, phospholipid dispersions, lamellar layers and the like. In these preparations, the peptide or nucleic acids to be delivered is incorporated as part of a liposome or embedded, alone or in conjunction with a molecule which binds to a receptor prevalent among lymphoid cells, such as monoclonal antibodies which bind to the CD45 antigen, or with other therapeutic or immunogenic compositions. Thus, liposomes either filled or decorated with a desired peptide or nucleic acids of the invention can be directed to the site of lymphoid cells, where the liposomes then deliver the peptide and nucleic acids compositions. Liposomes for use in accordance with the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al, 1980, and U.S. Pat. Nos. 4,235,871, 4,501,728, 4,837,028, and 5,019,369.

For targeting cells of the immune system, a ligand to be incorporated into the liposome can include, e.g., antibodies or fragments thereof specific for cell surface determinants of the desired immune system cells. A liposome suspension containing a peptide, polypeptide, conjugate, fusion protein, nucleic acid or vector may be administered intravenously, locally, topically, etc. in a dose which varies according to, inter alia, the manner of administration, the peptide, polypeptide, conjugate, fusion protein, nucleic acid, or vector being delivered, and the stage of the disease being treated. For example, liposomes carrying immunogenic polypeptides are known to elicit CTL responses in vivo (Reddy et al., 1992; Collins et al., 1992; Fries et al., 1992; Nabel et al., 1992).

After the liquid pharmaceutical composition is prepared, it is preferably lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is preferably administered to subjects using those methods that are known to those skilled in the art.

Another aspect of the present invention relates to conjugates of the isolated peptides or polypeptides or isolated multimeric peptides according to the present invention. Accordingly, the isolated peptides or polypeptides or isolated multimeric peptides according to the present invention may be an amino acid sequence conjugated at any amino acid sidechain or within the amino acid sequence with any chemical moiety, such as any therapeutic agent, such as any immunomodulating compound and such as any vaccine construct.

Another aspect of the present invention relates to isolated peptides or polypeptides or isolated multimeric peptides according to the present invention which are associated to a vehicle such as a virus, bacteria, or nanoparticle via covalent or non-covalent bonds, such as via conjugation or physical adsorption.

The terms “therapeutic agent”, such as “immunomodulating agent” as used herein, includes but is not limited to cytokines, such as interferons; monoclonal antibodies, such as anti-PD1 antibodies; as well as agents such as cyclophosphamide, Thalidomide, Levamisole, Lenalidomide, Mycobacterium obuense and other Mycobacterium sp.

### Use of the Peptides for Evaluating Immune Responses:

The peptides, polypeptides, conjugates, fusion proteins, combinations, nucleic acids or vectors according to the present invention may be used as diagnostic reagents.

For example, a peptide of the invention may be used to determine the susceptibility of a particular individual to a treatment regimen which employs the peptide or related peptides, and thus may be helpful in modifying an existing treatment protocol or in determining a prognosis for an affected individual. In addition, the peptides may also be used to predict which individuals will be at substantial risk for developing a chronic virus infection.

Accordingly, the present invention relates to a method of determining the outcome for a subject exposed to a virus, comprising the steps of determining whether the subject has an immune response to one or more peptides according to the present invention.

In a preferred embodiment of the invention, the peptides as described herein can be used as reagents to evaluate an immune response. The immune response to be evaluated can be induced by using as an immunogen any agent that may result in the production of antigen-specific CTLs or HTLs that recognize and bind to the peptide(s) to be employed as the reagent. The peptide reagent need not be used as the immunogen. Assay systems that can be used for such an analysis include relatively recent technical developments such as tetramers, staining for intracellular lymphokines and interferon release assays, or ELISPOT assays.

For example, a peptide of the invention may be used in a tetramer staining assay to assess peripheral blood mononuclear cells for the presence of antigen-specific CTLs following exposure to an antigen or an immunogen. The HLA-tetrameric complex is used to directly visualize antigen-specific CTLS (see, e.g., Ogg et al., 1998; and Altman et al., 1996) and determine the frequency of the antigen-specific CTL population in a sample of peripheral blood mononuclear cells. A tetramer reagent using a peptide of the invention may be generated as follows: a peptide that binds to an HLA molecule is refolded in the presence of the corresponding HLA heavy chain and beta2-microglobulin to generate a trimolecular complex. The complex is biotinylated at the carboxyl terminal end of the heavy chain at a site that was previously engineered into the protein. Tetramer formation is then induced by the addition of streptavidin. By means of fluorescently labeled streptavidin, the tetramer can be used to stain antigen-specific cells. The cells may then be identified, for example, by flow cytometry. Such an analysis may be used for diagnostic or prognostic purposes. Cells identified by the procedure can also be used for therapeutic purposes. As an alternative to tetramers also pentamers or dimers can be used (Current Protocols in Immunology (2000) unit 17.2 supplement 35)

Peptides of the invention may also be used as reagents to evaluate immune recall responses. (see, e.g., Bertoni et al., 1997 and Perma et al., 1991.). For example, patient PBMC samples from individuals with HCV infection may be analyzed for the presence of antigen-specific CTLs or HTLs using specific peptides. A blood sample containing mononuclear cells may be evaluated by cultivating the PBMCs and stimulating the cells with a peptide of the invention.

After an appropriate cultivation period, the expanded cell population may be analyzed, for example, for cytotoxic activity (CTL) or for HTL activity.

The peptides may also be used as reagents to evaluate the efficacy of a vaccine.

PBMCs obtained from a patient vaccinated with an immunogen may be analyzed using, for example, either of the methods described above. The patient is HLA typed, and peptide epitope reagents that recognize the allele-specific molecules present in that patient are selected for the analysis. The immunogenicity of the vaccine is indicated by the presence of epitope-specific CTLs and/or HTLs in the PBMC sample.

The peptides of the invention may also be used to make antibodies, using techniques well known in the art (see, e.g. CURRENT PROTOCOLS IN IMMUNOLOGY, Wiley/Greene, NY; and Antibodies A Laboratory Manual, Harlow and Lane, Cold Spring Harbor Laboratory Press, 1989). Such antibodies include those that recognize a peptide in the context of an HLA molecule, i.e., antibodies that bind to a peptide-MHC complex.

### Combinations and Conjugation of Peptides

Monomeric peptides according to two, three, four, five or more different aspects of the invention as set forth in the first to eleventh aspects can be used in combination, as set forth by the twelfth aspect.

In some embodiments, the combination comprises monomeric peptides from two, three, four, five or more different aspects as defined herein which are not linked to each other. For example, a composition may comprise a mixture of the monomeric peptides. One or more or all of the monomeric peptides in the combination or composition may optionally, however, be linked to another moiety as described in more detail below.

In some embodiments, the combination comprises monomeric peptides from two, three, four, five or more different aspects as defined herein, in which a first monomeric peptide is directly or indirectly associated with at least one second monomeric peptide. For example, the combination may comprise a sequence of amino acids of a first monomeric peptide which is directly or indirectly associated with the sequence of amino acids of at least a second monomeric polypeptide.

In certain embodiments a first monomeric peptide and the at least one second monomeric peptide are associated via a linker; the linker may comprise any peptide linker, or peptide spacer, such as a glycine, a lysine or an arginine linker/spacer, a polyhistidinyl tag, Protein G, and Protein A but it is also possible to use a bis-maleimide linker/spacer, a disulfide linker, or a polyethylene glycol (PEG) linker. In practice, any linker found useful in peptide chemistry is also useful as a linker according to the present invention. Thus, the invention contemplates the use of “simple” linear peptides which are conjugated or fused to each other, e.g. via a peptide segment or peptide bond in a monomeric polypeptide, but also peptide combinations where the individual peptides derived from a natural antigen are linked via non-peptide linkers. Use of multiple linker types are also within the scope of the present invention, and it is e.g. also a part of the invention to utilise linear peptides which include intrachain disulphide linkers.

In some embodiments, the combination comprises monomeric peptides from two, three, four, five or more different aspects as defined herein, in which a monomeric polypeptide from 10 to 80 amino acids in length comprises a first monomeric peptide segment and at least a second monomeric peptide segment. For example, a monomeric polypeptide of 10 to 80 amino acids in length may comprise the sequence of amino acids of a first monomeric peptide and the sequence of amino acids of at least a second monomeric polypeptide. In certain embodiments, a monomeric polypeptide from 10 to 80 amino acids in length comprises a first, a second and a third monomeric polypeptide as defined herein. For example, a monomeric polypeptide of 10 to 80 amino acids in length may comprise the sequences of amino acids of a first monomeric peptide, a second monomeric polypeptide, and a third monomeric polypeptide. The two, three, four, five or more monomeric peptide segments in a monomeric polypeptide may be consecutive in any order and may be flanked by or linked to another moiety as described in more detail below.

Typically, the full-length amino acid sequence of a monomeric polypeptide according to the invention differs from any segment of the amino acid sequence of SEQ ID NO:1 or SEQ ID NO:291 which has the same length. In some embodiments, a monomeric polypeptide of the invention differs from any such segment in SEQ ID NO:1 or SEQ ID NO:291 by at least two, such as at least three, such as at least four, such as at least five, such as at least six, such as at least seven, such as at least eight, such as at least nine, such as at least ten, such as at least fifteen, such as at least twenty, amino acid insertions, deletions and/or substitutions.

In some embodiments, the combination comprises monomeric peptides from two, three, four, five or more different aspects as defined herein, in which a composition comprises a mixture of (i) a first monomeric polypeptide from 10 to 80 amino acids in length comprising a first monomeric peptide segment, and (ii) a second monomeric polypeptide from 10 to 80 amino acids in length comprising a second monomeric polypeptide segment. In certain embodiments, the first or second polypeptide further comprises a third, a third and a fourth, or a third, fourth and fifth, monomeric peptide segment as defined herein. In certain embodiments, a composition may comprise a mixture of the first and the second monomeric polypeptides. Such a composition may also comprise one or more additional monomeric polypeptides. Particularly contemplated as first or second monomeric polypeptides are those exemplified in the eleventh aspect. One or more or all of the monomeric polypeptides may optionally be linked to another moiety as described in more detail below. A monomeric polypeptide of 10 to 80 amino acids in length may also comprise two, three, four, five or more copies of the same monomeric peptide, e.g., in the form of a tandem repeat.

In some embodiments, the combination comprises monomeric peptides from two, three, four, five or more different aspects as defined herein, in which a multimeric polypeptide comprises a first monomeric polypeptide from 10 to 80 amino acids in length comprising a first monomeric peptide segment and a second monomeric polypeptide from 10 to 80 amino acids in length comprising at least one second monomeric polypeptide segment, wherein the first and second monomeric polypeptides are covalently joined as described herein. In certain embodiments, the first or second polypeptide segments further comprises a third, a third and a fourth, or a third, fourth and fifth, monomeric peptide segment as defined herein. Such a composition may also comprise one or more additional polypeptides. One or more or all of the monomeric polypeptides may optionally be linked to another moiety as described in more detail below.

In any combination as described herein, the N- or C-terminal amino acid residue of any monomeric peptide or peptide segment may be linked to a sequence of amino acids selected from G, R, GG, GR, RG, RR, RRR, GGG, GGR, RGG, GRG, RRG, GRR, and RGR. In certain embodiments, the N- and C-terminal amino acid residue any monomeric peptide or peptide segment may be linked to a sequence of amino acids selected from G, R, GG, GR, RG, RR, RRR, GGG, GGR, RGG, GRG, RRG, GRR, and RGR. In certain embodiments, the N or C-terminal amino acid residue of at least the first monomeric peptide or peptide segment is linked to a sequence of amino acids independently selected from G, R, GG, GR, RG, RR, RRR, GGG, GGR, RGG, GRG, RRG, GRR, and RGR.

Particularly interesting peptide combinations of the invention are set forth in the preamble to the examples, in the section entitled “Specific embodiments of the invention.”

The following combinations of monomeric peptides according to different aspects are also particularly contemplated:
(a) a monomeric peptide according to the first aspect and a monomeric peptide according to the second aspect and,
(b) a monomeric peptide according to the first aspect and a monomeric peptide according to the third aspect and,
(c) a monomeric peptide according to the second aspect and a monomeric peptide according to the third aspect and,
(d) monomeric peptides according to the first, second and third aspects,
(e) monomeric peptides according to the first, second, third, fourth, fifth and sixth aspects,
(f) monomeric peptides according to the ninth, tenth and eleventh aspects,
(g) monomeric peptides according to the first, second, third and seventh aspects, such as according to the first, second, third and eighth aspects,
(h) monomeric peptides according to the first, second, third, and ninth aspects, such as according to the first, second, third and tenth aspects, such as according to the first, second, third and eleventh aspects.

In some specific embodiments, the polypeptide comprises at least one intrachain bond, such as a disulphide bond.

In some embodiments, the invention relates to a monomeric polypeptide of 10 to 80 amino acids in length, which monomeric polypeptide comprises
(a) a first monomeric peptide according to the first aspect as described herein,
(b) a second monomeric peptide according to the third aspect as described herein, and
(c) optionally, a third monomeric peptide according to the second aspect as described herein, wherein the N- or C-terminal of the monomeric polypeptide has a sequence of amino acids selected from G, R, GG, GR, RG, RR, RRR, GGG, GGR, RGG, GRG, RRG, GRR, and RGR.

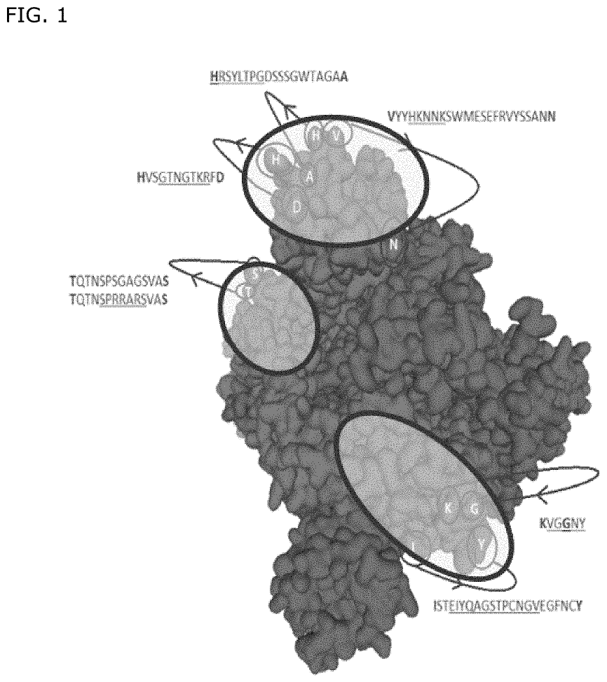

In some embodiments, the monomeric polypeptide is designed so as to comprise one or more monomeric peptides representing a surface site of interest, such as a receptor-binding domain of the spike protein (SEQ ID NO:1 and mutants thereof), a ganglioside-binding domain, and/or a furin cleavage site, as represented in FIG. 1.

In some embodiments, the invention relates to a monomeric polypeptide of 10 to 80 amino acids in length, in which
(a) the first monomeric peptide may comprise the sequence of amino acids NGVKGFNC identified as position 481-488 of SEQ ID NO:1 with an E484K amino acid substitution, or the sequence of amino acids STPSNGVE identified as position 477-493 with a C480S amino acid substitution;
(b) the second monomeric peptide may comprise the sequence of amino acids GVGYQP (SEQ ID NO:44); and
(c) the third monomeric peptide may comprise the sequence of amino acids KVGGNY (SEQ ID NO:35).

For example, the third monomeric polypeptide may comprise the sequence of amino acids LDSKVGGNY identified as position 441-449 of SEQ ID NO:1.

In some embodiments, the invention provides a polypeptide comprising or consisting of the sequence of amino acids RNGVKGFNCYFCLQSYGPTYGVGYQPNNLDSKVGGNYLYCRLFRYKGTQGR (SEQ ID NO:951), or a variant thereof comprising one, two, three, or four amino acid substitutions, insertions or deletions, which variant has no more than one amino acid substitution, insertion or deletion per three, such as per four, such as per five, such as per six consecutive amino acids of SEQ ID NO:951.

In some embodiments, the invention relates to a monomeric polypeptide of 10 to 80 amino acids in length, which monomeric polypeptide comprises
(a) a first monomeric peptide comprising the sequence of amino acids FYPRGQGVF (SEQ ID NO:292),
(b) a second monomeric peptide comprising the sequence of amino acids ATNTASWFR (SEQ ID NO:293),
(c) a third monomeric peptide comprising the sequence of amino acids FQFPRGQGI (SEQ ID NO:294), or
(d) a combination of (a) and (b), (a) and (c), (b) and (c), or (a) to (c).

In some embodiments, the invention relates to a polypeptide comprising or consisting of a sequence of amino acids RRFYPRGQGVFLEGATNTASWFRSRGFQFPRGQGIG (SEQ ID NO:950), or a variant thereof comprising one, two, three, or four amino acid substitutions, insertions or deletions, which variant has no more than one amino acid substitution, insertion or deletion per three, such as per four, such as per five, such as per six consecutive amino acids of SEQ ID NO:950.

In some embodiments, the invention relates to a polypeptide comprising or consisting of a sequence of amino acids QTQTNGSQSIIAGCGNLTTRTQKRFANGATWC (SEQ ID NO:952), or a variant thereof comprising one, two, three, or four amino acid substitutions, insertions or deletions, which variant has no more than one amino acid substitution, insertion or deletion per three, such as per four, such as per five, such as per six consecutive amino acids of the amino acid sequence of said SEQ ID NO.

In some embodiments, the invention relates to a polypeptide comprising or consisting of a sequence of amino acids selected from DCEGKYHKNNKSWCEAVHRSYITPG (SEQ ID NO:953), or a variant thereof comprising one, two, three, or four amino acid substitutions, insertions or deletions, which variant has no more than one amino acid substitution, insertion or deletion per three, such as per four, such as per five, such as per six consecutive amino acids of the amino acid sequence of said SEQ ID NO.

In some embodiments, the invention relates to a polypeptide comprising or consisting of a sequence of amino acids selected from TVRDPQTCDITESNKKFIPLGCGQLTPTWGRR (SEQ ID NO:954), or a variant thereof comprising one, two, three, or four amino acid substitutions, insertions or deletions, which variant has no more than one amino acid substitution, insertion or deletion per three, such as per four, such as per five, such as per six consecutive amino acids of the amino acid sequence of said SEQ ID NO.

In some embodiments, a monomeric polypeptide comprises or consists of an amino acid sequence selected from RRFYPRGQGVFLEGATNTASWFRSRGFQFPRGQGIG (SEQ ID NO:950), RNGVKGFNCYFCLQSYGPTYGVGYQPNNLDSKVGGNYLYCRLFRYKGTQGR (SEQ ID NO:951), QTQTNGSQSIIAGCGNLTTRTQKRFANGATWC (SEQ ID NO:952), DCEGKYHKNNKSWCEAVHRSYITPG (SEQ ID NO:953), TVRDPQTCDITESNKKFIPLGCGQLTPTWGRR (SEQ ID NO:954), or a variant thereof comprising one, two, three, or four amino acid substitutions, insertions or deletions, which variant has no more than one amino acid substitution, insertion or deletion per three, such as per four, such as per five, such as per six consecutive amino acids of said SEQ ID NO.

In some embodiments, a combination comprises monomeric polypeptides or multimeric polypeptides comprising or consisting of the amino acid sequences RRFYPRGQGVFLEGATNTASWFRSRGFQFPRGQGIG (SEQ ID NO:950) or a variant thereof, RNGVKGFNCYFCLQSYGPTYGVGYQPNNLDSKVGGNYLYCRLFRYKGTQGR (SEQ ID NO:951) or a variant thereof, QTQTNGSQSIIAGCGNLTTRTQKRFANGATWC (SEQ ID NO:952) or a variant thereof, DCEGKYHKNNKSWCEAVHRSYITPG (SEQ ID NO:953) or a variant thereof, and TVRDPQTCDITESNKKFIPLGCGQLTPTWGRR (SEQ ID NO:954) or a variant thereof, wherein said variant comprises one, two, three, or four amino acid substitutions, insertions or deletions, which variant has no more than one amino acid substitution, insertion or deletion per three, such as per four, such as per five, such as per six consecutive amino acids of said SEQ ID NO.

In some embodiments, a monomeric polypeptide comprises or consists of an amino acid sequence selected from RRGFKSYGVSPTKLNDSKVGGNYQNRLDSKVGGNY (SEQ ID NO:945), RRSTPSNGVERRGVEGFNENRFQPTNGRNRGVGYQP (SEQ ID NO:946), RRGASTEKSNRNGINITRQLLHAPATVRTNGVGYG (SEQ ID NO:947), RRKSTNLVGGATVTGPGGGVKNKSVGGPLSETK (SEQ ID NO:948), RRFYPRGQGVFLEGATNTASWFRSRGFQFPRGQGIG (SEQ ID NO:950), or a variant thereof comprising one, two, three, or four amino acid substitutions, insertions or deletions, which variant has no more than one amino acid substitution, insertion or deletion per three, such as per four, such as per five, such as per six consecutive amino acids of said SEQ ID NO.

In some embodiments, a combination comprises monomeric polypeptides or multimeric polypeptides comprising or consisting of the amino acid sequences RRGFKSYGVSPTKLNDSKVGGNYQNRLDSKVGGNY (SEQ ID NO:945) or a variant thereof, RRSTPSNGVERRGVEGFNENRFQPTNGRNRGVGYQP (SEQ ID NO:946) or a variant thereof, RRGASTEKSNRNGINITRQLLHAPATVRTNGVGYG (SEQ ID NO:947) or a variant thereof, RRKSTNLVGGATVTGPGGGVKNKSVGGPLSETK (SEQ ID NO:948) or a variant thereof, and RRFYPRGQGVFLEGATNTASWFRSRGFQFPRGQGIG (SEQ ID NO:950) or a variant thereof, wherein said variant comprises one, two, three, or four amino acid substitutions, insertions or deletions, which variant has no more than one amino acid substitution, insertion or deletion per three, such as per four, such as per five, such as per six consecutive amino acids of said SEQ ID NO.

In certain embodiments, at least one of the first and at least one second peptides in the peptide combination comprises an N- or C-terminal modification, such as an amidation, acylation, or acetylation.

Since the peptide combinations are contemplated as vaccine agents or diagnostic agents, they are in certain embodiments coupled to a carrier molecule, such as an immunogenic carrier. The peptides of the peptide combinations may thus be linked to other molecules either as recombinant fusions (e.g. via CLIP technology) or through chemical linkages in an oriented (e.g. using heterobifunctional cross-linkers) or nonoriented fashion. Linking to carrier molecules such as for example diphtheria toxin, latex beads (convenient in diagnostic and prognostic embodiments), and magnetic beads (also convenient in diagnostic and prognostic embodiments), polylysine constructs etc, are all possible according to the invention.

The immunogenic carrier is conveniently selected from carrier proteins such as those conventionally used in the art (e.g. diphtheria or tetanus toxoid, KLH etc.), but it is also possible to use shorter peptides (T-helper epitopes) which can induce T-cell immunity in larger proportions of a population. Details about such T-helper epitopes can e.g. be found in WO 00/20027, which is hereby incorporated by reference herein—all immunolgic carriers and “promiscuous” (i.e. universal) T-helper epitopes discussed therein are useful as immunogenic carriers in the present invention.

In certain embodiments, the carrier is a virus like particle, i.e. a particle sharing properties with virions without being infectious. Such virus-like particles may be provided chemically (e.g. Jennings and Bachmann Ann Rev Pharmacol. Toxicol. 2009. 49:303-26 Immunodrugs: Therapeutic VLP-based vaccines for chronic diseases) or using cloning techniques to generate fusion proteins (e.g. Peabody et al. J. Mol. Biol. 2008; 380: 252-63. Immunogenic display of diverse peptides on virus-like particles of RNA phage MS2). Another example is “Remune”, an HIV vaccine originally made by Immune Response Corporation, which consists of formalin inactivated HIV that has been irradiated to destroy the viral genome.

In an embodiment, a nucleic acid is encoding one or more monomeric peptides of the multimeric, such as dimeric, peptide according to the invention, where the encoded first peptide and the encoded at least one second peptide of a multimeric peptide are associated via a peptide linker, including a peptide spacer, and/or a disulphide bridge. The peptide linker/spacer is typically selected from the group consisting of a glycine, an arginine, a lysine linker/spacer, or a glycine-lysine linker/spacer, but any peptide linker known in the art may be useful. The term peptide linker thus also is intended to denote coupling between the first and second peptide via a peptide bond. A peptide linker that links a first and second peptide by standard peptide bonds may also be referred to as a peptide spacer. Also, the first and second peptides may be linked via a peptide linker and a disulphide bond, as is the case when an intrachain disulphide bond is established.

In one embodiment, the nucleic acid according to the invention encodes the peptide combination, which is coupled (by fusion) to a carrier molecule, such as an immunogenic carrier; useful carriers are discussed above.

In some embodiments the linker is selected from the group consisting of a bis-maleimide linker, a disulfide linker, a polyethylene glycol (PEG) linker, a glycine linker/spacer, a lysine linker/spacer, and an arginine linker/spacer.

In some embodiments the multimeric peptide, such as a dimeric peptide, contain a linker in the free amino group of the N-terminal of a monomeric peptide linking said monomeric peptide to another monomeric peptide.

In some embodiments the multimeric peptide, such as a dimeric peptide contain a linker in the free carboxyl group of the C-terminal of a monomeric peptide linking said monomeric peptide to another monomeric peptide.

At least two options for such linkers are described in A. R Jacobson et al, J. Med. Chem. 1989, 32, 1708-1717 and in D Giannotti et al, Journal of Medicinal Chemistry, 2000, Vol. 43, No. 22, the disclosures of which is hereby incorporated by reference.

Alternatively a link between the N-termini of peptides may be established by reacting with Br—(CH₂)ₙ—Br.

The length of the linker may be varied by the addition of glycine residues, for example Fmoc-NH—CH₂CH₂—NH-Gly-NH₂ may be used.

Methods of synthesizing monomeric and multimeric peptides of the present disclosure are known in the art. The peptides of the present disclosure can, for example, be prepared by chemical synthesis methods, which are well known in the art. See, e.g., Peptide Synthesis and Applications 2nd edition, Jensen, K. J.; Tofteng Shelton, P.; Pedersen, S. L. Eds. Springer: New York, Heidelberg, Dordrecht, London, 2013.

A multimeric, such as dimeric peptide, such as a heterodimeric peptide may be synthesized by, but are not restricted to the following protocol:

To the peptidyl resin containing deblocked Asp or Glu residue (monomer 1) is added HBTU, DIPEA and Trt-amino PEG amine in DMF. The mixture is allowed to couple overnight. The resin is filtered from the solution and washed by standard protocol. The Trt group is removed from the Trt-PEGylated peptide. The monomer 2 containing deblocked Asp or Glu residue is then coupled to the exposed amino group using HBTU and DIPEA. After cleavage the desired product is purified using any suitable technique to give the desired multimeric peptide.

Another exemplary chemical synthesis method is Solid Phase Peptide Synthesis (SPPS), which may be carried out on an automatic peptide synthesizer (e.g., Biotage Initiator+Alstra, Biotage Syro II, or Activotec Activo-P11), using Fmoc-protected amino acids for peptide elongation. Removal of Fmoc group can be performed using 20-40% piperidine in DMF, and coupling performed using 4 eq of corresponding amino acid, 4 eq of HBTU, 4 eq of HOBt, and 8 eq of DIPEA at temperatures from ambient to 75° C. Crude peptides may then be deprotected and cleaved from the resin through treatment with TFA/H2O/iPr3SiH followed by precipitation in cold ether followed by purification, e.g., using reverse phase chromatography.

In some embodiments the isolated monomeric peptide or polypeptide contains intramolecular bonds, such as in the form of intramolecular Cys-Cys bonds, also known as disulphide bonds.

It is to be understood that the “intramolecular bond”, used interchangeably with “intrachain bond”, is a bond between two different amino acids within the same peptide chain, which however is not necessarily adjacent to each other in the peptide sequence. Accordingly, in some embodiments, the isolated monomeric peptide, monomeric polypeptide or multimeric polypeptide according to the invention may contain both intramolecular bonds within one or more of the monomers, as well as an intermolecular bond between two separate peptide chains of the multimeric peptide, such as a dimer. This intramolecular bond may be in the form of Cys-Cys bonds formed with cysteine residues within the same peptide sequence. In some embodiments the monomer contains an intramolecular bond derived from a Lys residue or other amino acid residue, such as a Ser, Cys, Asp or Glu that make the bond, such as a thioether bond or an oxime bond or through a PEG linker, to an amino acid residue on the other monomer peptide sequence.

In some embodiments, the amino acid sequence of a monomeric peptide or monomeric polypeptide can be modified so as to introduce, via amino acid insertion or substitution, amino acid residues providing for one or more intrachain bonds, e.g., for the purpose of achieving a desired conformation or folding of the monomeric peptide or monomeric polypeptide. For example, in a monomeric peptide or monomeric polypeptide comprising one cysteine residue, a second cysteine residue can be introduced at a desired location in the monomeric peptide or monomeric polypeptide by amino acid insertion or substitution.

Method for Synthesis of Multimeric Peptides with PolyLys or MAPS:

PolyLys or MAPS (multiple antigen peptides)—has been extensively used over the last 20 years as a carrier protein to produce strong immunogenic response. The MAP system utilizes a peptidyl core of three or more radially branched lysine core to form a backbone for which the epitope sequences of interest can be built parallel using standard solid-phase chemistry.

The MAP system is a commercial product available from several companies such as AnaSpec, Bio-synthesis Inc. and others. The product, as offered in the catalogue only allows attachment of two (identical) peptide sequence to the polyLys core. It is however possible also to link two different peptide sequences by using different protecting groups for alfa- and epsylon-amino functional groups of lysine on the two different peptide sequences.

Use of the MAP system has been described in references including: Wang, C. Y et al. “Long-term high-titer neutralizing activity induced by octameric synthetic HIV antigen” Science 254, 285-288 (1991). Posnett, D. et al. “A novel method for producing anti-peptide antibodies” J. Biol. Chem. 263, 1719-1725 (1988), and in Tam, J. P. “Synthetic peptide vaccine design: synthesis and properties of a high-density multiple antigenic peptide system” PNAS USA 85, 5409-5413 (1988).

The MAP system could also be prepared by chemical (thioether, oxime, hydrazone) ligation of appropriately functionalized tetra- or octavalent polylysine constructs with the peptide antigen. By the use of this chemical ligation, the two peptide sequences being linked together would not have to be identical as they are synthesized separately.

Additionally a novel application of the MAP-based system is to synthesize on solid support a “probe” containing a poly(ethylene glycol) (PEG) chain in the dendritic arms of MAP.

Use of the MAP system will increase the size of a multimeric complex and may increase the immunogenic response.

Methods for the Synthesis of Multimeric Peptides Using PEG:

Suitable Multi-Arm Activated PEG to be used for a PEG linker are commercially available, e.g. a compound with the following structure:

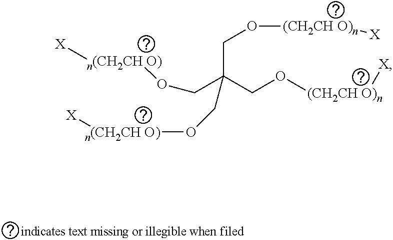

wherein X may be ethanethiol —CH2CH2SH (could be used to form S—S bridge with the epitope or a thioether link) or propylamine —CH2CH2CH2NH2, among others. These handles preferably allow for the linking of two identical peptide sequences and may be seen as a poly-monomeric epitope presenting construct. One could, however, anchor a dimer (two epitopes linked together) to the PEG above.

Method for Synthesis of Peptide-Poly-L-Lys (PLL)-Polyethylene Glycol (PEG) Construct:

Peptide-PLL-PEG constructs, may be synthesized by, but are not restricted to the following protocol:

Fmoc-Poly-L-Lys-resin (a commercial product) is de-protected with 20% piperifine-DMF. Fmoc-NH-PEG4-COOH, in a mixed solvent of CH2Cl2-NMP is added followed by HBTU and DIPEA and the reaction is allowed to proceed for 24 h. The resultant pegylated poly-L-Lys-resin is washed and the pegylation step is repeated. The reaction is monitored by Kaiser's ninhydrin test until a negative reading is obtained. After de-protection of Fmoc group, four identical peptide chains are synthesized directly on the branched poly-L-Lys-polyethylene glycol core by a stepwise solid-phase procedure. All residues activated with HBTU and DIPEA are allowed to couple for 2 h. The coupling is monitored by Kaiser's ninhydrin test and is repeated if needed. After cleavage the desired product is purified using any suitable technique to give the desired peptide-construct.

Peptide Slow-Release Formulations:

A suitable peptide slow-release formulation is as disclosed in WO2013083459, WO2012160212, or WO2013083459.

### SPECIFIC EMBODIMENTS OF THE INVENTION

As stated above the present invention relates to specific sequences of Corona virus, in particular Wuhan seafood market pneumonia virus isolate Wuhan-Hu-1, in particular a monomeric peptide consisting of at least 5, such as at least 6 consecutive amino acids of the sequence of amino acids STPCNGVEGFNC identified as position 477-488 of SEQ ID NO:1; or a variant thereof containing one, two, three, or four amino acid substitution, which variant has not more than one amino acid substitution per three, such as per four, such as per five, such as per six consecutive amino acids of said sequence STPCNGVEGFNC, at least 5, such as at least 6 consecutive amino acids of the sequence of amino acids LDSKVGGNY identified as position 441-449 of SEQ ID NO:1; or a variant thereof containing one, two, or three amino acid substitutions, which variant has not more than one amino acid substitution per three, such as per four, such as per five, such as per six consecutive amino acids of said sequence LDSKVGGNY, at least 5, such as at least 6 consecutive amino acids of the sequence of amino acids FQPTNGVGYQP identified as position 497-507 of SEQ ID NO:1; or a variant thereof containing one, two, or three amino acid substitution, which variant has not more than one amino acid substitution per three, such as per four, such as per five, such as per six consecutive amino acids of said sequence FQPTNGVGYQP, and monomeric peptide consisting of a sequence of amino acids as defined in any one of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 36, SEQ ID NO: 37, SEQ ID NO: 45, SEQ ID NO: 46, or SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, or SEQ ID NO: 290; or a variant thereof containing one or two amino acid substitutions, or one amino acid deletion.

In some specific embodiments this monomeric peptide is at least 5, 6, 7, 8, 9, or 10 amino acids in length, such as 6 or 7 amino acids in length.

In some specific embodiments this monomeric peptide is not more than 12, 11, 10, 9, 8, 7, 6, or 5 amino acids in length.

In some specific embodiments this monomeric peptide has an overall net charge equal to or above 0, such as above 1, 2, 3, 4, or 5.

In some specific embodiments this monomeric peptide is capable of inducing a humoral immune response.

In some specific embodiments this monomeric peptide comprises at least one amino acid selected from a Cys, a Lys, an Asp, and a Glu residue, or derivatives thereof.

In some specific embodiments this monomeric peptide has delayed proteolytic degradation in the N-terminal, such as by incorporation of the first 1, 2, or 3 amino acids in the N-terminal in the D-form, or by incorporation of the first 1, 2, or 3 amino acids in the N-terminal in beta or gamma form.

Also as mentioned above the present invention relates to a monomeric polypeptide from 10 to 80 amino acids in length, which polypeptide comprises or consist of two, three or four consecutive sequences of amino acids as independently defined herein, which two, three or four consecutive sequences of amino acids is optionally separated by or having in the N- or C-terminal of the polypeptide a sequence of amino acids selected from G, R, GG, GR, RG, RR, RRR, GGG, GGR, RGG, GRG, RRG, GRR, or RGR.

In some specific embodiments this polypeptide is a cyclic polypeptide.

In some specific embodiments this monomeric polypeptide is of 10-80 amino acids, such as of 11-80 amino acids, such as of 12-80 amino acids, such as of 13-80 amino acids, such as of 14-80 amino acids, such as of 15-80 amino acids, such as of 16-80 amino acids, such as of 17-80 amino acids, such as of 18-80 amino acids, such as of 19-80 amino acids, such as of 20-80 amino acids, such as of 21-80 amino acids, such as of 22-80 amino acids, such as of 23-80 amino acids, such as of 24-80 amino acids, such as of 25-80 amino acids, such as of 26-80 amino acids, such as of 27-80 amino acids, such as of 28-80 amino acids, such as of 29-80 amino acids, such as of 30-80 amino acids, such as of 31-80 amino acids, such as of 32-80 amino acids, such as of 33-80 amino acids, such as of 34-80 amino acids, such as of 35-80 amino acids, such as of 36-80 amino acids, such as of 37-80 amino acids, such as of 38-80 amino acids, such as of 39-80 amino acids, such as of 40-80 amino acids, such as of 42-80 amino acids, such as of 44-80 amino acids, such as of 46-80 amino acids, such as of 48-80 amino acids, such as of 50-80 amino acids, such as of 52-80 amino acids, such as of 54-80 amino acids, such as of 56-80 amino acids, such as of 58-80 amino acids, such as of 59-80 amino acids, such as of 60-80 amino acids, such as of 61-80 amino acids, such as of 62-80 amino acids, such as of 63-80 amino acids, such as of 64-80 amino acids, such as of 65-80 amino acids, such as of 66-80 amino acids, such as of 67-80 amino acids, such as of 68-80 amino acids, such as of 69-80 amino acids, such as of 70-80 amino acids in length.

In some specific embodiments this monomeric polypeptide is of 10-78 amino acids, such as 10-76 amino acids, such as 10-74 amino acids, such as 10-72 amino acids, such as 10-70 amino acids, such as 10-68 amino acids, such as 10-66 amino acids, such as 10-64 amino acids, such as 10-62 amino acids, such as 10-60 amino acids, such as 10-58 amino acids, such as 10-56 amino acids, such as 10-54 amino acids, such as 10-52 amino acids, such as 10-50 amino acids, such as 10-48 amino acids, such as 10-46 amino acids, such as 10-44 amino acids, such as 10-42 amino acids, such as 10-40 amino acids, such as 10-39 amino acids, such as 10-38 amino acids, such as 10-37 amino acids, such as 10-36 amino acids, such as 10-35 amino acids, such as 10-34 amino acids, such as 10-33 amino acids, such as 10-32 amino acids, such as 10-31 amino acids, such as 10-30 amino acids, such as 10-29 amino acids, such as 10-28 amino acids, such as 10-27 amino acids, such as 10-26 amino acids, such as 10-25 amino acids, such as 10-24 amino acids, such as 10-23 amino acids, such as 10-22 amino acids, such as 10-21 amino acids, such as 10-20 amino acids, such as 10-19 amino acids, such as 10-18 amino acids, such as 10-17 amino acids, such as 10-16 amino acids, such as 10-15 amino acids, such as 10-14 amino acids, such as 10-13 amino acids, such as 10-12 amino acids, such as 10-11 amino acids in length.

In some specific embodiments this monomeric polypeptide consist of not more than about 70 amino acids, such as not more than about 65 amino acids, such as not more than about 60 amino acids, such as not more than about 55 amino acids, such as not more than about 50 amino acids, such as not more than about 45 amino acids, such as not more than about 40 amino acids, such as not more than about 38 amino acids, such as not more than about 36 amino acids, such as not more than about 34 amino acids, such as not more than about 32 amino acids, such as not more than about 30 amino acids, such as not more than about 28 amino acids, such as not more than about 26 amino acids, such as not more than about 24 amino acids, such as not more than about 22 amino acids, such as not more than about 20 amino acids, such as not more than about 18 amino acids, such as not more than about 16 amino acids, such as not more than about 14 amino acids, such as not more than about 12 amino acids, such as not more than about 10 amino acids in length.

In some specific embodiments this monomeric polypeptide consist of at least about 10 amino acids, such as at least about 12 amino acids, such as at least about 14 amino acids, such as at least about 16 amino acids, such as at least about 18 amino acids, such as at least about 20 amino acids, such as at least about 22 amino acids, such as at least about 24 amino acids, such as at least about 26 amino acids, such as at least about 28 amino acids, such as at least about 30 amino acids, such as at least about 32 amino acids, such as at least about 34 amino acids, such as at least about 36 amino acids, such as at least about 38 amino acids, such as at least about 40 amino acids, such as at least about 45 amino acids, such as at least about 50 amino acids, such as at least about 55 amino acids, such as at least about 60 amino acids, such as at least about 65 amino acids, such as at least about 70 amino acids, such as at least about 75 amino acids in length.

In some specific embodiments the overall net charge of this polypeptide is equal to or above 0, such as above 1, 2, 3, 4, or 5.

In some specific embodiments this monomeric polypeptide is capable of inducing a humoral immune response.

Other aspects of the invention relates to a multimeric peptide, such as a dimeric peptide comprising at least a first monomeric peptide or polypeptide as defined herein, covalently joined to at least a second monomeric peptide or polypeptide independently as defined herein, the monomeric polypeptides being covalently joined, such as joined by a disulfide (S—S) bond between a Cys residue in each monomeric peptide.

In some specific embodiments in this multimeric, such as dimeric peptide the first and the second monomeric peptides are identical in sequence.

In some specific embodiments in this multimeric, such as dimeric peptide the first and the second monomeric peptides are different in sequence.

Sequences

Complete spike protein sequence of Wuhan seafood market pneumonia virus isolate Wuhan-Hu-1 (SEQ ID NO:1) (highlighted in bold underline are some particular positions in the sequence):

| | | |
|---|---|---|
| 1 | MFVFLVLLPLVSSQCVNLTTRTQLPPAYTNSFTRGVYYPDKVFRSSVLHSTQDLFLPFFS | 60 |
| 61 | NVTWFHAIHVSGTNGTKRFDNPVLPFNDGVYFASTEKSNIIRGWIFGTTLDSKTQSLLIV | 120 |
| 121 | NNATNVVIKVCEFQFCNDPFLGVYYHKNNKSWMESEFRVYSSANNCTFEYVSQPFLMDLE | 180 |
| 181 | GKQGNFKNLREFVFKNIDGYFKIYSKHTPINLVRDLPQGFSALEPLVDLPIGINITRFQT | 240 |
| 241 | LLALHRSYLTPGDSSSGWTAGAAAYYVGYLQPRTFLLKYNENGTITDAVDCALDPLSETK | 300 |
| 301 | CTLKSFTVEKGIYQTSNFRVQPTESIVRFPNITNLCPFGEVFNATRFASVYAWNRKRISN | 360 |
| 361 | CVADYSVLYNSASFSTFKCYGVSPTKLNDLCFTNVYADSFVIRGDEVRQIAPGQTGKIAD | 420 |
| 421 | YNYKLPDDFTGCVIAWNSNNLDSKVGGNYNYLYRLFRKSNLKPFERDISTEIYQAGSTPC | 480 |
| 481 | NGVEGFNCYFPLQSYGFQPTNGVGYQPYRVVVLSFELLHAPATVCGPKKSTNLVKNKCVN | 540 |
| 541 | FNFNGLTGTGVLTESNKKFLPFQQFGRDIADTTDAVRDPQTLEILDITPCSFGGVSVITP | 600 |
| 601 | GTNTSNQVAVLYQDVNCTEVPVAIHADQLTPTWRVYSTGSNVFQTRAGCLIGAEHVNNSY | 660 |
| 661 | ECDIPIGAGICASYQTQTNSPRRARSVASQSIIAYTMSLGAENSVAYSNNSIAIPTNFTI | 720 |
| 721 | SVTTEILPVSMTKTSVDCTMYICGDSTECSNLLLQYGSFCTQLNRALTGIAVEQDKNTQE | 780 |
| 781 | VFAQVKQIYKTPPIKDFGGFNFSQILPDPSKPSKRSFIEDLLFNKVTLADAGFIKQYGDC | 840 |
| 841 | LGDIAARDLICAQKFNGLTVLPPLLTDEMIAQYTSALLAGTITSGWTFGAGAALQIPFAM | 900 |
| 901 | QMAYRFNGIGVTONVLYENQKLIANQFNSAIGKIQDSLSSTASALGKLQDVVNQNAQALN | 960 |
| 961 | TLVKQLSSNFGAISSVLNDILSRLDKVEAEVQIDRLITGRLQSLQTYVTQQLIRAAEIRA | 1020 |
| 1021 | SANLAATKMSECVLGQSKRVDFCGKGYHLMSFPQSAPHGVVFLHVTYVPAQEKNFTTAPA | 1080 |
| 1081 | ICHDGKAHFPREGVFVSNGTHWFVTQRNFYEPQIITTDNTFVSGNCDVVIGIVNNTVYDP | 1140 |
| 1141 | LQPELDSFKEELDKYFKNHTSPDVDLGDISGINASVVNIQKEIDRLNEVAKNLNESLIDL | 1200 |
| 1201 | QELGKYEQYIKWPWYIWLGFIAGLIAIVMVTIMLCCMTSCCSCLKGCCSCGSCCKFDEDD | 1260 |
| 1261 | SEPVLKGVKLHYT | 1273 |

Nucleocapsid phosphoprotein sequence; protein_id=“QHD43423.2”; GenBank: MN908947.3 (SEQ ID NO: 291)

| | | |
|---|---|---|
| 1 | MSDNGPQNQRNAPRITFGGPSDSTGSNQNGERSGARSKQRRPQGLPNNTASWFTALTQHG | 60 |
| 61 | KEDLKFPRGQGVPINTNSSPDDQIGYYRRATRRIRGGDGKMKDLSPRWYFYYLGTGPEAG | 120 |
| 121 | LPYGANKDGIIWVATEGALNTPKDHIGTRNPANNAAIVLQLPQGTTLPKGFYAEGSRGGS | 180 |
| 181 | QASSRSSSRSRNSSRNSTPGSSRGTSPARMAGNGGDAALALLLLDRLNQLESKMSGKGQQ | 240 |
| 241 | QQGQTVTKKSAAEASKKPRQKRTATKAYNVTQAFGRRGPEQTQGNFGDQELIRQGTDYKH | 300 |
| 301 | WPQIAQFAPSASAFFGMSRIGMEVTPSGTWLTYTGAIKLDDKDPNFKDQVILLNKHIDAY | 360 |
| 361 | KTFPPTEPKKDKKKKADETQALPQRQKKQQTVTLLPAADLDDFSKQLQQSMSSADSTQA | 419 |

TABLE 1
Corona specific non-human like sequences
(Underlined are positions of the sequence
especially relevant to the present invention):

| AMINO | | | AMINO | | |
|---|---|---|---|---|---|
| ACID | | | ACID | REF | |
| SEQUENCE | REF NO. | SEQ ID NO: | SEQUENCE | NO. | SEQ ID NO: |
| ASTEKS | 17 | SEQ ID NO: 2 | MSDNGP | | SEQ ID NO: 295 |
| STEKSN | 18 | SEQ ID NO: 3 | NAPRIT | | SEQ ID NO: 296 |
| LGVYYH | 32 | SEQ ID NO: 4 | RITFGG | | SEQ ID NO: 297 |
| GVYYHK | 33 | SEQ ID NO: 5 | ITFGGP | | SEQ ID NO: 298 |
| VYSSAN | 34 | SEQ ID NO: 6 | GGPSDS | | SEQ ID NO: 299 |
| SANNCT | 35 | SEQ ID NO: 7 | GPSDST | | SEQ ID NO: 300 |
| YVSQPF | 36 | SEQ ID NO: 8 | DSTGSN | | SEQ ID NO: 301 |
| LEGKQG | 38 | SEQ ID NO: 9 | STGSNQ | | SEQ ID NO: 302 |
| GKQGNF | 40 | SEQ ID NO: 10 | TGSNQN | | SEQ ID NO: 303 |
| GINITR | 48 | SEQ ID NO: 11 | GSNQNG | | SEQ ID NO: 304 |
| KCTLKS | 57 | SEQ ID NO: 12 | SNQNGE | | SEQ ID NO: 305 |
| TESIVR | 61 | SEQ ID NO: 13 | GARSKQ | | SEQ ID NO: 306 |
| ITNLCP | 64 | SEQ ID NO: 14 | SKQRRP | | SEQ ID NO: 307 |
| LCPFGE | 66 | SEQ ID NO: 15 | LPNNTA | | SEQ ID NO: 308 |
| ATRFAS | 67 | SEQ ID NO: 16 | NTASWF | | SEQ ID NO: 309 |
| TRFASV | 68 | SEQ ID NO: 17 | TASWFT | | SEQ ID NO: 310 |
| SNCVAD | 69 | SEQ ID NO: 18 | SWFTAL | | SEQ ID NO: 311 |
| VLYNSA | 70 | SEQ ID NO: 19 | FTALTQ | | SEQ ID NO: 312 |
| FKCYGV | 71 | SEQ ID NO: 20 | LTQHGK | | SEQ ID NO: 313 |
| KCYGVS | 72 | SEQ ID NO: 21 | TQHGKE | | SEQ ID NO: 314 |
| CYGVSP | 73 | SEQ ID NO: 22 | HGKEDL | | SEQ ID NO: 315 |
| YGVSPT | 74 | SEQ ID NO: 23 | DLKFPR | | SEQ ID NO: 316 |
| GVSPTK | 75 | SEQ ID NO: 24 | LKFPRG | | SEQ ID NO: 317 |
| VSPTKL | 76 | SEQ ID NO: 25 | KFPRGQ | | SEQ ID NO: 318 |
| ADSFVI | 77 | SEQ ID NO: 26 | FPRGQG | | SEQ ID NO: 319 |
| QIAPGQ | 78 | SEQ ID NO: 27 | PRGQGV | | SEQ ID NO: 320 |
| TGKIAD | 80 | SEQ ID NO: 28 | RGQGVP | | SEQ ID NO: 321 |
| GKIADY | 81 | SEQ ID NO: 29 | PINTNS | | SEQ ID NO: 322 |
| KIADYN | 82 | SEQ ID NO: 30 | INTNSS | | SEQ ID NO: 323 |
| DDFTGC | 83 | SEQ ID NO: 31 | TNSSPD | | SEQ ID NO: 324 |
| FTGCVI | 84 | SEQ ID NO: 32 | QIGYYR | | SEQ ID NO: 325 |
| LDSKVG | 86 | SEQ ID NO: 33 | YRRATR | | SEQ ID NO: 326 |
| DSKVGG | 87 | SEQ ID NO: 34 | TRRIRG | | SEQ ID NO: 327 |
| KVGGNY | 88 | SEQ ID NO: 35 | RIRGGD | | SEQ ID NO: 328 |
| KSNLKP | 90 | SEQ ID NO: 36 | IRGGDG | | SEQ ID NO: 329 |
| EIYQAG | 94 | SEQ ID NO: 37 | RGGDGK | | SEQ ID NO: 330 |
| STPCNG | 95 | SEQ ID NO: 38 | WYFYYL | | SEQ ID NO: 331 |
| TPCNGV | 96 | SEQ ID NO: 39 | YFYYLG | | SEQ ID NO: 332 |
| PCNGVE | 97 | SEQ ID NO: 40 | LGTGPE | | SEQ ID NO: 333 |
| GVEGFN | 98 | SEQ ID NO: 41 | GANKDG | | SEQ ID NO: 334 |
| FQPTNG | 100 | SEQ ID NO: 42 | ANKDGI | | SEQ ID NO: 335 |
| TNGVGY | 101 | SEQ ID NO: 43 | KDGIIW | | SEQ ID NO: 336 |
| GVGYQP | 102 | SEQ ID NO: 44 | TEGALN | | SEQ ID NO: 337 |
| LLHAPA | 103 | SEQ ID NO: 45 | EGALNT | | SEQ ID NO: 338 |
| LHAPAT | 104 | SEQ ID NO: 46 | NTPKDH | | SEQ ID NO: 339 |
| HAPATV | 105 | SEQ ID NO: 47 | PKDHIG | | SEQ ID NO: 340 |
| STPCNGVEGFSC | | SEQ ID NO: 48 | HIGTRN | | SEQ ID NO: 341 |
| STPCNGVEGFNC | | SEQ ID NO: 49 | GTRNPA | | SEQ ID NO: 342 |
| STPCNGVEGFQC | | SEQ ID NO: 50 | AAIVLQ | | SEQ ID NO: 343 |
| PCNPADFNCYFP | | SEQ ID NO: 51 | AIVLQL | | SEQ ID NO: 344 |
| PCQGLEFNCYWP | | SEQ ID NO: 52 | PQGTTL | | SEQ ID NO: 345 |
| PCQGLEFQCYWP | | SEQ ID NO: 53 | GTTLPK | | SEQ ID NO: 346 |
| PCQGLEFSCYWP | | SEQ ID NO: 54 | TTLPKG | | SEQ ID NO: 347 |
| STPSNG | | SEQ ID NO: 55 | FYAEGS | | SEQ ID NO: 348 |
| VEGFSC | | SEQ ID NO: 56 | SQASSR | | SEQ ID NO: 349 |
| VEGFNC | | SEQ ID NO: 57 | RSRNSS | | SEQ ID NO: 350 |
| VEGFQC | | SEQ ID NO: 58 | SRNSTP | | SEQ ID NO: 351 |
| PCNPAD | | SEQ ID NO: 59 | RNSTPG | | SEQ ID NO: 352 |
| FNCYFP | | SEQ ID NO: 60 | NSTPGS | | SEQ ID NO: 353 |
| PCQGLE | | SEQ ID NO: 61 | GSSRGT | | SEQ ID NO: 354 |
| FNCYWP | | SEQ ID NO: 62 | SRGTSP | | SEQ ID NO: 355 |
| FQCYWP | | SEQ ID NO: 63 | RGTSPA | | SEQ ID NO: 356 |
| FSCYWP | | SEQ ID NO: 64 | GTSPAR | | SEQ ID NO: 357 |
| PLVSSQ | 1 | SEQ ID NO: 65 | RMAGNG | | SEQ ID NO: 358 |
| SSQCVN | 2 | SEQ ID NO: 66 | MAGNGG | | SEQ ID NO: 359 |
| VNLTTR | 3 | SEQ ID NO: 67 | AGNGGD | | SEQ ID NO: 360 |
| LTTRTQ | 4 | SEQ ID NO: 68 | GNGGDA | | SEQ ID NO: 361 |
| TRTQLP | 5 | SEQ ID NO: 69 | GDAALA | | SEQ ID NO: 362 |
| PAYTNS | 6 | SEQ ID NO: 70 | DRLNQL | | SEQ ID NO: 363 |
| VFRSSV | 7 | SEQ ID NO: 71 | NQLESK | | SEQ ID NO: 364 |
| VLHSTQ | 8 | SEQ ID NO: 72 | ESKMSG | | SEQ ID NO: 365 |
| LFLPFF | 9 | SEQ ID NO: 73 | MSGKGQ | | SEQ ID NO: 366 |
| SNVTWF | 10 | SEQ ID NO: 74 | SGKGQQ | | SEQ ID NO: 367 |
| VSGTNG | 11 | SEQ ID NO: 75 | QGQTVT | | SEQ ID NO: 368 |
| SGTNGT | 12 | SEQ ID NO: 76 | GQTVTK | | SEQ ID NO: 369 |
| TNGTKR | 13 | SEQ ID NO: 77 | TVTKKS | | SEQ ID NO: 370 |
| NGTKRF | 14 | SEQ ID NO: 78 | KSAAEA | | SEQ ID NO: 371 |
| RFDNPV | 15 | SEQ ID NO: 79 | AEASKK | | SEQ ID NO: 372 |
| VYFAST | 16 | SEQ ID NO: 80 | EASKKP | | SEQ ID NO: 373 |
| IRGWIF | 19 | SEQ ID NO: 81 | PRQKRT | | SEQ ID NO: 374 |
| WIFGTT | 20 | SEQ ID NO: 82 | KRTATK | | SEQ ID NO: 375 |
| FGTTLD | 21 | SEQ ID NO: 83 | RTATKA | | SEQ ID NO: 376 |
| TTLDSK | 22 | SEQ ID NO: 84 | VTQAFG | | SEQ ID NO: 377 |
| LDSKTQ | 23 | SEQ ID NO: 85 | TQAFGR | | SEQ ID NO: 378 |
| DSKTQS | 24 | SEQ ID NO: 86 | AFGRRG | | SEQ ID NO: 379 |
| SKTQSL | 25 | SEQ ID NO: 87 | GRRGPE | | SEQ ID NO: 380 |
| KTQSLL | 26 | SEQ ID NO: 88 | TQGNFG | | SEQ ID NO: 381 |
| VNNATN | 27 | SEQ ID NO: 89 | GNFGDQ | | SEQ ID NO: 382 |
| ATNVVI | 28 | SEQ ID NO: 90 | DQELIR | | SEQ ID NO: 383 |
| CEFQFC | 29 | SEQ ID NO: 91 | LIRQGT | | SEQ ID NO: 384 |
| FCNDPF | 30 | SEQ ID NO: 92 | RQGTDY | | SEQ ID NO: 385 |
| CNDPFL | 31 | SEQ ID NO: 93 | QGTDYK | | SEQ ID NO: 386 |
| VSQPFL | 37 | SEQ ID NO: 94 | AQFAPS | | SEQ ID NO: 387 |
| EGKQGN | 39 | SEQ ID NO: 95 | PSASAF | | SEQ ID NO: 388 |
| SKHTPI | 41 | SEQ ID NO: 96 | EVTPSG | | SEQ ID NO: 389 |
| LVRDLP | 42 | SEQ ID NO: 97 | VTPSGT | | SEQ ID NO: 390 |
| RDLPQG | 43 | SEQ ID NO: 98 | YTGAIK | | SEQ ID NO: 391 |
| PQGFSA | 44 | SEQ ID NO: 99 | AIKLDD | | SEQ ID NO: 392 |
| GFSALE | 45 | SEQ ID NO: 100 | HIDAYK | | SEQ ID NO: 393 |
| VDLPIG | 46 | SEQ ID NO: 101 | AYKTFP | | SEQ ID NO: 394 |
| PIGINI | 47 | SEQ ID NO: 102 | YKTFPP | | SEQ ID NO: 395 |
| LTPGDS | 49 | SEQ ID NO: 103 | KKKADE | | SEQ ID NO: 396 |
| TPGDSS | 50 | SEQ ID NO: 104 | KKADET | | SEQ ID NO: 397 |
| AAYYVG | 51 | SEQ ID NO: 105 | QALPQR | | SEQ ID NO: 398 |
| GYLQPR | 52 | SEQ ID NO: 106 | SADSTQ | | SEQ ID NO: 399 |
| ALDPLS | 53 | SEQ ID NO: 107 | ADSTQA | | SEQ ID NO: 400 |
| LDPLSE | 54 | SEQ ID NO: 108 | ATNTASWFD | | SEQ ID NO: 401 |
| PLSETK | 55 | SEQ ID NO: 109 | ATNTASWFF | | SEQ ID NO: 402 |
| SETKCT | 56 | SEQ ID NO: 110 | ATNTASWFK | | SEQ ID NO: 403 |
| TVEKGI | 58 | SEQ ID NO: 111 | ATNTASWFN | | SEQ ID NO: 404 |
| TSNFRV | 59 | SEQ ID NO: 112 | ATNTASWFR | | SEQ ID NO: 405 |
| FRVQPT | 60 | SEQ ID NO: 113 | ATNTASWFS | | SEQ ID NO: 406 |
| SIVRFP | 62 | SEQ ID NO: 114 | GGNTASWFR | | SEQ ID NO: 407 |
| PNITNL | 63 | SEQ ID NO: 115 | GGNTASWFS | | SEQ ID NO: 408 |
| NLCPFG | 65 | SEQ ID NO: 116 | GTNTASWFR | | SEQ ID NO: 409 |
| APGQTG | 79 | SEQ ID NO: 117 | GTNTASWFS | | SEQ ID NO: 410 |
| GCVIAW | 85 | SEQ ID NO: 118 | GNTASWFTG | | SEQ ID NO: 411 |
| GGNYNY | 89 | SEQ ID NO: 119 | GTTASWFTG | | SEQ ID NO: 412 |
| PFERDI | 91 | SEQ ID NO: 120 | TNTASWFTG | | SEQ ID NO: 413 |
| ISTEIY | 92 | SEQ ID NO: 121 | KGFTALTQS | | SEQ ID NO: 414 |
| STEIYQ | 93 | SEQ ID NO: 122 | RGFTALTQS | | SEQ ID NO: 415 |
| PLQSYG | 99 | SEQ ID NO: 123 | ASTQHGKER | | SEQ ID NO: 416 |
| APATVC | 106 | SEQ ID NO: 124 | EGTQHGKEN | | SEQ ID NO: 417 |
| PATVCG | 107 | SEQ ID NO: 125 | ESTQHGKEN | | SEQ ID NO: 418 |
| ATVCGP | 108 | SEQ ID NO: 126 | FSTQHGKEN | | SEQ ID NO: 419 |
| TVCGPK | 109 | SEQ ID NO: 127 | GGTQHGKEN | | SEQ ID NO: 420 |
| VCGPKK | 110 | SEQ ID NO: 128 | GHTQHGKEN | | SEQ ID NO: 421 |
| GPKKST | 111 | SEQ ID NO: 129 | IGTQHGKEN | | SEQ ID NO: 422 |
| PKKSTN | 112 | SEQ ID NO: 130 | ISTQHGKEN | | SEQ ID NO: 423 |
| KKSTNL | 113 | SEQ ID NO: 131 | ITTQHGKEN | | SEQ ID NO: 424 |
| KSTNLV | 114 | SEQ ID NO: 132 | LTTQHGKEN | | SEQ ID NO: 425 |
| VKNKCV | 115 | SEQ ID NO: 133 | MGTQHGKEN | | SEQ ID NO: 426 |
| FNFNGL | 116 | SEQ ID NO: 134 | NGTQHGKEN | | SEQ ID NO: 427 |
| FNGLTG | 117 | SEQ ID NO: 135 | QSTQHGKEN | | SEQ ID NO: 428 |
| NGLTGT | 118 | SEQ ID NO: 136 | QSTQHGKER | | SEQ ID NO: 429 |
| TGTGVL | 119 | SEQ ID NO: 137 | RHTQHGKEN | | SEQ ID NO: 430 |
| GTGVLT | 120 | SEQ ID NO: 138 | RSTQHGKEN | | SEQ ID NO: 431 |
| GVLTES | 121 | SEQ ID NO: 139 | RTTQHGKEN | | SEQ ID NO: 432 |
| TESNKK | 122 | SEQ ID NO: 140 | STTQHGKEN | | SEQ ID NO: 433 |
| PFQQFG | 123 | SEQ ID NO: 141 | STTQHGKER | | SEQ ID NO: 434 |
| QQFGRD | 124 | SEQ ID NO: 142 | TTTQHGKEN | | SEQ ID NO: 435 |
| ADTTDA | 125 | SEQ ID NO: 143 | YGTQHGKEN | | SEQ ID NO: 436 |
| DTTDAV | 126 | SEQ ID NO: 144 | DALKFPRGQ | | SEQ ID NO: 437 |
| TTDAVR | 127 | SEQ ID NO: 145 | EYLKFPRGH | | SEQ ID NO: 438 |
| DAVRDP | 128 | SEQ ID NO: 146 | PALKFPRGQ | | SEQ ID NO: 439 |
| AVRDPQ | 129 | SEQ ID NO: 147 | QYLKFPRGH | | SEQ ID N 0+440 |
| DITPCS | 130 | SEQ ID NO: 148 | SNLKFPRGH | | SEQ ID NO: 441 |
| ITPCSF | 131 | SEQ ID NO: 149 | SNLKFPRGQ | | SEQ ID NO: 442 |
| TPCSFG | 132 | SEQ ID NO: 150 | ADKFPRGQL | | SEQ ID NO: 443 |
| FGGVSV | 133 | SEQ ID NO: 151 | ADKFPRGQS | | SEQ ID NO: 444 |
| GGVSVI | 134 | SEQ ID NO: 152 | AKKFPRGQH | | SEQ ID NO: 445 |
| SVITPG | 135 | SEQ ID NO: 153 | AKKFPRGQL | | SEQ ID NO: 446 |
| ITPGTN | 136 | SEQ ID NO: 154 | AKKFPRGQN | | SEQ ID NO: 447 |
| TPGTNT | 137 | SEQ ID NO: 155 | AKKFPRGQP | | SEQ ID NO: 448 |
| PGTNTS | 138 | SEQ ID NO: 156 | ALKFPRGQL | | SEQ ID NO: 449 |
| TSNQVA | 139 | SEQ ID NO: 157 | ALKFPRGQP | | SEQ ID NO: 450 |
| VAVLYQ | 140 | SEQ ID NO: 158 | ALKFPRGQS | | SEQ ID NO: 451 |
| QLTPTW | 141 | SEQ ID NO: 159 | CSKFPRGQH | | SEQ ID NO: 452 |
| STGSNV | 142 | SEQ ID NO: 160 | CSKFPRGQL | | SEQ ID NO: 453 |
| GSNVFQ | 143 | SEQ ID NO: 161 | CSKFPRGQP | | SEQ ID NO: 454 |
| FQTRAG | 144 | SEQ ID NO: 162 | CSKFPRGQS | | SEQ ID NO: 455 |
| QTRAGC | 145 | SEQ ID NO: 163 | DCKFPRGQH | | SEQ ID NO: 456 |
| RAGCLI | 146 | SEQ ID NO: 164 | DCKFPRGQP | | SEQ ID NO: 457 |
| AGCLIG | 147 | SEQ ID NO: 165 | FKKFPRGQH | | SEQ ID NO: 458 |
| AEHVNN | 148 | SEQ ID NO: 166 | FKKFPRGQL | | SEQ ID NO: 459 |
| IPIGAG | 149 | SEQ ID NO: 167 | FKKFPRGQN | | SEQ ID NO: 460 |
| AGICAS | 150 | SEQ ID NO: 168 | FKKFPRGQS | | SEQ ID NO: 461 |
| SYQTQT | 151 | SEQ ID NO: 169 | GAKFPRGQC | | SEQ ID NO: 462 |
| QTQTNS | 152 | SEQ ID NO: 170 | GAKFPRGQH | | SEQ ID NO: 463 |
| TQTNSP | 153 | SEQ ID NO: 171 | GAKFPRGQL | | SEQ ID NO: 464 |
| TNSPRR | 154 | SEQ ID NO: 172 | GAKFPRGQS | | SEQ ID NO: 465 |
| SQSIIA | 155 | SEQ ID NO: 173 | GCKFPRGQH | | SEQ ID NO: 466 |
| MSLGAE | 156 | SEQ ID NO: 174 | GCKFPRGQL | | SEQ ID NO: 467 |
| SIAIPT | 157 | SEQ ID NO: 175 | GCKFPRGQN | | SEQ ID NO: 468 |
| TNFTIS | 158 | SEQ ID NO: 176 | GCKFPRGQP | | SEQ ID NO: 469 |
| SVTTEI | 159 | SEQ ID NO: 177 | GCKFPRGQS | | SEQ ID NO: 470 |
| VTTEIL | 160 | SEQ ID NO: 178 | GSKFPRGQC | | SEQ ID NO: 471 |
| TTEILP | 161 | SEQ ID NO: 179 | GSKFPRGQH | | SEQ ID NO: 472 |
| TKTSVD | 162 | SEQ ID NO: 180 | GSKFPRGQP | | SEQ ID NO: 473 |
| KTSVDC | 163 | SEQ ID NO: 181 | GSKFPRGQQ | | SEQ ID NO: 474 |
| TSVDCT | 164 | SEQ ID NO: 182 | GSKFPRGQS | | SEQ ID NO: 475 |
| YICGDS | 165 | SEQ ID NO: 183 | HAKFPRGQC | | SEQ ID NO: 476 |
| ICGDST | 166 | SEQ ID NO: 184 | HAKFPRGQH | | SEQ ID NO: 477 |
| CGDSTE | 167 | SEQ ID NO: 185 | HAKFPRGQL | | SEQ ID NO: 478 |
| GDSTEC | 168 | SEQ ID NO: 186 | HAKFPRGQS | | SEQ ID NO: 479 |
| DSTECS | 169 | SEQ ID NO: 187 | HDKFPRGQH | | SEQ ID NO: 480 |
| TECSNL | 170 | SEQ ID NO: 188 | HDKFPRGQL | | SEQ ID NO: 481 |
| LQYGSF | 171 | SEQ ID NO: 189 | HDKFPRGQP | | SEQ ID NO: 482 |
| GSFCTQ | 172 | SEQ ID NO: 190 | HDKFPRGQS | | SEQ ID NO: 483 |
| SFCTQL | 173 | SEQ ID NO: 191 | HQKFPRGQG | | SEQ ID NO: 484 |
| CTQLNR | 174 | SEQ ID NO: 192 | HQKFPRGQH | | SEQ ID NO: 485 |
| RALTGI | 175 | SEQ ID NO: 193 | HQKFPRGQP | | SEQ ID NO: 486 |
| ALTGIA | 176 | SEQ ID NO: 194 | HQKFPRGQS | | SEQ ID NO: 487 |
| VFAQVK | 177 | SEQ ID NO: 195 | HSKFPRGQH | | SEQ ID NO: 488 |
| QVKQIY | 178 | SEQ ID NO: 196 | HSKFPRGQL | | SEQ ID NO: 489 |
| QIYKTP | 179 | SEQ ID NO: 197 | HSKFPRGQP | | SEQ ID NO: 490 |
| YKTPPI | 180 | SEQ ID NO: 198 | HSKFPRGQS | | SEQ ID NO: 491 |
| PIKDFG | 181 | SEQ ID NO: 199 | IAKFPRGQC | | SEQ ID NO: 492 |
| FGGFNF | 182 | SEQ ID NO: 200 | IAKFPRGQH | | SEQ ID NO: 493 |
| GGFNFS | 183 | SEQ ID NO: 201 | IAKFPRGQL | | SEQ ID NO: 494 |
| PSKPSK | 184 | SEQ ID NO: 202 | IAKFPRGQP | | SEQ ID NO: 495 |
| SKPSKR | 185 | SEQ ID NO: 203 | IAKFPRGQS | | SEQ ID NO: 496 |
| KPSKRS | 186 | SEQ ID NO: 204 | KAKFPRGQH | | SEQ ID NO: 497 |
| PSKRSF | 187 | SEQ ID NO: 205 | KAKFPRGQL | | SEQ ID NO: 498 |
| NKVTLA | 188 | SEQ ID NO: 206 | KAKFPRGQP | | SEQ ID NO: 499 |
| KVTLAD | 189 | SEQ ID NO: 207 | KAKFPRGQS | | SEQ ID NO: 500 |
| DAGFIK | 190 | SEQ ID NO: 208 | KKKFPRGQH | | SEQ ID NO: 501 |
| AGFIKQ | 191 | SEQ ID NO: 209 | KKKFPRGQL | | SEQ ID NO: 502 |
| GFIKQY | 192 | SEQ ID NO: 210 | KKKFPRGQN | | SEQ ID NO: 503 |
| FIKQYG | 193 | SEQ ID NO: 211 | KKKFPRGQP | | SEQ ID NO: 504 |
| DCLGDI | 194 | SEQ ID NO: 212 | KKKFPRGQS | | SEQ ID NO: 505 |
| CLGDIA | 195 | SEQ ID NO: 213 | KQKFPRGQH | | SEQ ID NO: 506 |
| LICAQK | 196 | SEQ ID NO: 214 | KQKFPRGQP | | SEQ ID NO: 507 |
| AQKFNG | 197 | SEQ ID NO: 215 | KQKFPRGQS | | SEQ ID NO: 508 |
| QKFNGL | 198 | SEQ ID NO: 216 | KSKFPRGQH | | SEQ ID NO: 509 |
| NGLTVL | 199 | SEQ ID NO: 217 | KSKFPRGQL | | SEQ ID NO: 510 |
| LLTDEM | 200 | SEQ ID NO: 218 | KSKFPRGQP | | SEQ ID NO: 511 |
| SGWTFG | 201 | SEQ ID NO: 219 | KSKFPRGQS | | SEQ ID NO: 512 |
| GWTFGA | 202 | SEQ ID NO: 220 | LAKFPRGQC | | SEQ ID NO: 513 |
| WTFGAG | 203 | SEQ ID NO: 221 | LAKFPRGQH | | SEQ ID NO: 514 |
| TFGAGA | 204 | SEQ ID NO: 222 | LAKFPRGQL | | SEQ ID NO: 515 |
| IPFAMQ | 205 | SEQ ID NO: 223 | LAKFPRGQP | | SEQ ID NO: 516 |
| MAYRFN | 206 | SEQ ID NO: 224 | LAKFPRGQS | | SEQ ID NO: 517 |
| RFNGIG | 207 | SEQ ID NO: 225 | LSKFPRGQC | | SEQ ID NO: 518 |
| NGIGVT | 208 | SEQ ID NO: 226 | LSKFPRGQH | | SEQ ID NO: 519 |
| GIGVTQ | 209 | SEQ ID NO: 227 | LSKFPRGQL | | SEQ ID NO: 520 |
| IGVTQN | 210 | SEQ ID NO: 228 | LSKFPRGQP | | SEQ ID NO: 521 |
| QKLIAN | 211 | SEQ ID NO: 229 | LSKFPRGQS | | SEQ ID NO: 522 |
| KLIANQ | 212 | SEQ ID NO: 230 | NAKFPRGQH | | SEQ ID NO: 523 |
| LIANQF | 213 | SEQ ID NO: 231 | NAKFPRGQL | | SEQ ID NO: 524 |
| QFNSAI | 214 | SEQ ID NO: 232 | NAKFPRGQN | | SEQ ID NO: 525 |
| NSAIGK | 215 | SEQ ID NO: 233 | NAKFPRGQP | | SEQ ID NO: 526 |
| AIGKIQ | 216 | SEQ ID NO: 234 | NAKFPRGQS | | SEQ ID NO: 527 |
| ALGKLQ | 217 | SEQ ID NO: 235 | NKKFPRGQG | | SEQ ID NO: 528 |
| NQNAQA | 218 | SEQ ID NO: 236 | NKKFPRGQH | | SEQ ID NO: 529 |
| ALNTLV | 219 | SEQ ID NO: 237 | NKKFPRGQL | | SEQ ID NO: 530 |
| NFGAIS | 220 | SEQ ID NO: 238 | NKKFPRGQN | | SEQ ID NO: 531 |
| FGAISS | 221 | SEQ ID NO: 239 | NKKFPRGQP | | SEQ ID NO: 532 |
| AISSVL | 222 | SEQ ID NO: 240 | NKKFPRGQS | | SEQ ID NO: 533 |
| ILSRLD | 223 | SEQ ID NO: 241 | NLKFPRGQG | | SEQ ID NO: 534 |
| DRLITG | 224 | SEQ ID NO: 242 | NLKFPRGQL | | SEQ ID NO: 535 |
| ITGRLQ | 225 | SEQ ID NO: 243 | NLKFPRGQP | | SEQ ID NO: 536 |
| IRASAN | 226 | SEQ ID NO: 244 | NLKFPRGQS | | SEQ ID NO: 537 |
| NLAATK | 227 | SEQ ID NO: 245 | NSKFPRGQH | | SEQ ID NO: 538 |
| CVLGQS | 228 | SEQ ID NO: 246 | NSKFPRGQL | | SEQ ID NO: 539 |
| VLGQSK | 229 | SEQ ID NO: 247 | NSKFPRGQP | | SEQ ID NO: 540 |
| LGQSKR | 230 | SEQ ID NO: 248 | NSKFPRGQS | | SEQ ID NO: 541 |
| GQSKRV | 231 | SEQ ID NO: 249 | PCKFPRGQC | | SEQ ID NO: 542 |
| QSKRVD | 232 | SEQ ID NO: 250 | PCKFPRGQG | | SEQ ID NO: 543 |
| RVDFCG | 233 | SEQ ID NO: 251 | PCKFPRGQH | | SEQ ID NO: 544 |
| FCGKGY | 234 | SEQ ID NO: 252 | PCKFPRGQL | | SEQ ID NO: 545 |
| FPQSAP | 235 | SEQ ID NO: 253 | PCKFPRGQN | | SEQ ID NO: 546 |
| QSAPHG | 236 | SEQ ID NO: 254 | PCKFPRGQP | | SEQ ID NO: 547 |
| SAPHGV | 237 | SEQ ID NO: 255 | PCKFPRGQS | | SEQ ID NO: 548 |
| APHGVV | 238 | SEQ ID NO: 256 | PDKFPRGQP | | SEQ ID NO: 549 |
| HGVVFL | 239 | SEQ ID NO: 257 | PHKFPRGQH | | SEQ ID NO: 550 |
| VPAQEK | 240 | SEQ ID NO: 258 | PKKFPRGQG | | SEQ ID NO: 551 |
| KNFTTA | 241 | SEQ ID NO: 259 | PKKFPRGQH | | SEQ ID NO: 552 |
| FTTAPA | 242 | SEQ ID NO: 260 | PKKFPRGQL | | SEQ ID NO: 553 |
| CHDGKA | 243 | SEQ ID NO: 261 | PKKFPRGQN | | SEQ ID NO: 554 |
| HDGKAH | 244 | SEQ ID NO: 262 | PKKFPRGQP | | SEQ ID NO: 555 |
| KAHFPR | 245 | SEQ ID NO: 263 | PKKFPRGQS | | SEQ ID NO: 556 |
| GVFVSN | 246 | SEQ ID NO: 264 | PLKFPRGQL | | SEQ ID NO: 557 |
| SNGTHW | 247 | SEQ ID NO: 265 | PLKFPRGQN | | SEQ ID NO: 558 |
| NGTHWF | 248 | SEQ ID NO: 266 | PLKFPRGQP | | SEQ ID NO: 559 |
| GTHWFV | 249 | SEQ ID NO: 267 | PLKFPRGQS | | SEQ ID NO: 560 |
| QRNFYE | 250 | SEQ ID NO: 268 | QAKFPRGQC | | SEQ ID NO: 561 |
| FYEPQI | 251 | SEQ ID NO: 269 | QAKFPRGQH | | SEQ ID NO: 562 |
| YEPQII | 252 | SEQ ID NO: 270 | QAKFPRGQL | | SEQ ID NO: 563 |
| TFVSGN | 253 | SEQ ID NO: 271 | QAKFPRGQS | | SEQ ID NO: 564 |
| SGNCDV | 254 | SEQ ID NO: 272 | QDKFPRGQL | | SEQ ID NO: 565 |
| GIVNNT | 255 | SEQ ID NO: 273 | QQKFPRGQG | | SEQ ID NO: 566 |
| NNTVYD | 256 | SEQ ID NO: 274 | QQKFPRGQH | | SEQ ID NO: 567 |
| QPELDS | 257 | SEQ ID NO: 275 | QQKFPRGQN | | SEQ ID NO: 568 |
| PELDSF | 258 | SEQ ID NO: 276 | QQKFPRGQP | | SEQ ID NO: 569 |
| LDSFKE | 259 | SEQ ID NO: 277 | QQKFPRGQS | | SEQ ID NO: 570 |
| TSPDVD | 260 | SEQ ID NO: 278 | SAKFPRGQC | | SEQ ID NO: 571 |
| RLNEVA | 261 | SEQ ID NO: 279 | SAKFPRGQG | | SEQ ID NO: 572 |
| NEVAKN | 262 | SEQ ID NO: 280 | SAKFPRGQH | | SEQ ID NO: 573 |
| YIKWPW | 263 | SEQ ID NO: 281 | SAKFPRGQL | | SEQ ID NO: 574 |
| LIAIVM | 264 | SEQ ID NO: 282 | SAKFPRGQP | | SEQ ID NO: 575 |
| SCLKGC | 265 | SEQ ID NO: 283 | SAKFPRGQS | | SEQ ID NO: 576 |
| CLKGCC | 266 | SEQ ID NO: 284 | SLKFPRGQG | | SEQ ID NO: 577 |
| CCKFDE | 267 | SEQ ID NO: 285 | SLKFPRGQL | | SEQ ID NO: 578 |
| EPVLKG | 268 | SEQ ID NO: 286 | SLKFPRGQP | | SEQ ID NO: 579 |
| KGVKLH | 269 | SEQ ID NO: 287 | SLKFPRGQS | | SEQ ID NO: 580 |
| GVKLHY | 270 | SEQ ID NO: 288 | TLKFPRGQL | | SEQ ID NO: 581 |
| ATVTGP | 271 | SEQ ID NO: 289 | TLKFPRGQN | | SEQ ID NO: 582 |
| VKNKSV | 272 | SEQ ID NO: 290 | TLKFPRGQP | | SEQ ID NO: 583 |
| FYPRGQGVF | | SEQ ID NO: 292 | TLKFPRGQS | | SEQ ID NO: 584 |
| ATNTASWFR | | SEQ ID NO: 293 | TQKFPRGQP | | SEQ ID NO: 585 |
| FQFPRGQGI | | SEQ ID NO: 294 | TQKFPRGQQ | | SEQ ID NO: 586 |
| VAKFPRGQH | | SEQ ID NO: 587 | DHPRGQGVM | | SEQ ID NO: 761 |
| VAKFPRGQL | | SEQ ID NO: 588 | DHPRGQGVQ | | SEQ ID NO: 762 |
| VAKFPRGQP | | SEQ ID NO: 589 | EFPRGQGVE | | SEQ ID NO: 763 |
| VAKFPRGQS | | SEQ ID NO: 590 | EFPRGQGVF | | SEQ ID NO: 764 |
| VSKFPRGQH | | SEQ ID NO: 591 | EFPRGQGVG | | SEQ ID NO: 765 |
| VSKFPRGQL | | SEQ ID NO: 592 | EFPRGQGVI | | SEQ ID NO: 766 |
| VSKFPRGQP | | SEQ ID NO: 593 | EFPRGQGVM | | SEQ ID NO: 767 |
| VSKFPRGQQ | | SEQ ID NO: 594 | EFPRGQGVQ | | SEQ ID NO: 768 |
| VSKFPRGQS | | SEQ ID NO: 595 | ETPRGQGVG | | SEQ ID NO: 769 |
| YAKFPRGQC | | SEQ ID NO: 596 | ETPRGQGVQ | | SEQ ID NO: 770 |
| YAKFPRGQH | | SEQ ID NO: 597 | FYPRGQGVF | | SEQ ID NO: 771 |
| YAKFPRGQL | | SEQ ID NO: 598 | FYPRGQGVG | | SEQ ID NO: 772 |
| YAKFPRGQP | | SEQ ID NO: 599 | FYPRGQGVI | | SEQ ID NO: 773 |
| YAKFPRGQS | | SEQ ID NO: 600 | FYPRGQGVQ | | SEQ ID NO: 774 |
| YDKFPRGQL | | SEQ ID NO: 601 | GVPRGQGVE | | SEQ ID NO: 775 |
| YDKFPRGQP | | SEQ ID NO: 602 | GYPRGQGVF | | SEQ ID NO: 776 |
| YDKFPRGQS | | SEQ ID NO: 603 | GYPRGQGVG | | SEQ ID NO: 777 |
| YKKFPRGQH | | SEQ ID NO: 604 | GYPRGQGVI | | SEQ ID NO: 778 |
| YKKFPRGQL | | SEQ ID NO: 605 | HHPRGQGVM | | SEQ ID NO: 779 |
| YKKFPRGQN | | SEQ ID NO: 606 | HYPRGQGVE | | SEQ ID NO: 780 |
| YKKFPRGQP | | SEQ ID NO: 607 | KFPRGQGVQ | | SEQ ID NO: 781 |
| YKKFPRGQS | | SEQ ID NO: 608 | KYPRGQGVE | | SEQ ID NO: 782 |
| YQKFPRGQP | | SEQ ID NO: 609 | LTPRGQGVG | | SEQ ID NO: 783 |
| YQKFPRGQS | | SEQ ID NO: 610 | LTPRGQGVM | | SEQ ID NO: 784 |
| YSKFPRGQH | | SEQ ID NO: 611 | LTPRGQGVQ | | SEQ ID NO: 785 |
| YSKFPRGQL | | SEQ ID NO: 612 | MAPRGQGVQ | | SEQ ID NO: 786 |
| YSKFPRGQS | | SEQ ID NO: 613 | PYPRGQGVF | | SEQ ID NO: 787 |
| AGFPRGQGC | | SEQ ID NO: 614 | PYPRGQGVI | | SEQ ID NO: 788 |
| AGFPRGQGD | | SEQ ID NO: 615 | QHPRGQGVG | | SEQ ID NO: 789 |
| AGFPRGQGE | | SEQ ID NO: 616 | QHPRGQGVM | | SEQ ID NO: 790 |
| AGFPRGQGG | | SEQ ID NO: 617 | QHPRGQGVQ | | SEQ ID NO: 791 |
| AGFPRGQGI | | SEQ ID NO: 618 | QYPRGQGVE | | SEQ ID NO: 792 |
| AGFPRGQGK | | SEQ ID NO: 619 | QYPRGQGVF | | SEQ ID NO: 793 |
| AGFPRGQGN | | SEQ ID NO: 620 | QYPRGQGVG | | SEQ ID NO: 794 |
| AGFPRGQGQ | | SEQ ID NO: 621 | QYPRGQGVI | | SEQ ID NO: 795 |
| AKFPRGQGN | | SEQ ID NO: 622 | QYPRGQGVQ | | SEQ ID NO: 796 |
| ASFPRGQGC | | SEQ ID NO: 623 | RFPRGQGVA | | SEQ ID NO: 797 |
| CGFPRGQGC | | SEQ ID NO: 624 | RFPRGQGVE | | SEQ ID NO: 798 |
| CGFPRGQGD | | SEQ ID NO: 625 | RFPRGQGVF | | SEQ ID NO: 799 |
| CGFPRGQGE | | SEQ ID NO: 626 | RFPRGQGVG | | SEQ ID NO: 800 |
| CGFPRGQGF | | SEQ ID NO: 627 | RFPRGQGVI | | SEQ ID NO: 801 |
| CGFPRGQGG | | SEQ ID NO: 628 | RFPRGQGVM | | SEQ ID NO: 802 |
| CGFPRGQGI | | SEQ ID NO: 629 | RFPRGQGVQ | | SEQ ID NO: 803 |
| CGFPRGQGK | | SEQ ID NO: 630 | SAPRGQGVM | | SEQ ID NO: 804 |
| CGFPRGQGN | | SEQ ID NO: 631 | SAPRGQGVQ | | SEQ ID NO: 805 |
| CGFPRGQGQ | | SEQ ID NO: 632 | SFPRGQGVE | | SEQ ID NO: 806 |
| CVFPRGQGC | | SEQ ID NO: 633 | SFPRGQGVG | | SEQ ID NO: 807 |
| CVFPRGQGD | | SEQ ID NO: 634 | STPRGQGVM | | SEQ ID NO: 808 |
| CVFPRGQGE | | SEQ ID NO: 635 | STPRGQGVN | | SEQ ID NO: 809 |
| CVFPRGQGN | | SEQ ID NO: 636 | STPRGQGVQ | | SEQ ID NO: 810 |
| CVFPRGQGQ | | SEQ ID NO: 637 | TAPRGQGVM | | SEQ ID NO: 811 |
| DKFPRGQGG | | SEQ ID NO: 638 | TAPRGQGVQ | | SEQ ID NO: 812 |
| DKFPRGQGQ | | SEQ ID NO: 639 | TTPRGQGVQ | | SEQ ID NO: 813 |
| DVFPRGQGD | | SEQ ID NO: 640 | TVPRGQGVE | | SEQ ID NO: 814 |
| DVFPRGQGF | | SEQ ID NO: 641 | VHPRGQGVM | | SEQ ID NO: 815 |
| DVFPRGQGN | | SEQ ID NO: 642 | VTPRGQGVM | | SEQ ID NO: 816 |
| DVFPRGQGQ | | SEQ ID NO: 643 | VTPRGQGVQ | | SEQ ID NO: 817 |
| EQFPRGQGD | | SEQ ID NO: 644 | WYPRGQGVF | | SEQ ID NO: 818 |
| EQFPRGQGG | | SEQ ID NO: 645 | WYPRGQGVG | | SEQ ID NO: 819 |
| EQFPRGQGK | | SEQ ID NO: 646 | WYPRGQGVI | | SEQ ID NO: 820 |
| EQFPRGQGN | | SEQ ID NO: 647 | YFPRGQGVE | | SEQ ID NO: 821 |
| FEFPRGQGI | | SEQ ID NO: 648 | YFPRGQGVG | | SEQ ID NO: 822 |
| FQFPRGQGD | | SEQ ID NO: 649 | YFPRGQGVI | | SEQ ID NO: 823 |
| FQFPRGQGE | | SEQ ID NO: 650 | YFPRGQGVQ | | SEQ ID NO: 824 |
| FQFPRGQGF | | SEQ ID NO: 651 | AKRGQGVPT | | SEQ ID NO: 825 |
| FQFPRGQGG | | SEQ ID NO: 652 | AMRGQGVPG | | SEQ ID NO: 826 |
| FQFPRGQGI | | SEQ ID NO: 653 | AMRGQGVPH | | SEQ ID NO: 827 |
| FQFPRGQGK | | SEQ ID NO: 654 | AMRGQGVPS | | SEQ ID NO: 828 |
| FQFPRGQGN | | SEQ ID NO: 655 | HMRGQGVPT | | SEQ ID NO: 829 |
| FVFPRGQGC | | SEQ ID NO: 656 | IQRGQGVPS | | SEQ ID NO: 830 |
| FVFPRGQGD | | SEQ ID NO: 657 | LQRGQGVPS | | SEQ ID NO: 831 |
| FVFPRGQGE | | SEQ ID NO: 658 | PKRGQGVPC | | SEQ ID NO: 832 |
| FVFPRGQGN | | SEQ ID NO: 659 | PKRGQGVPT | | SEQ ID NO: 833 |
| FVFPRGQGQ | | SEQ ID NO: 660 | SKRGQGVPT | | SEQ ID NO: 834 |
| GDFPRGQGE | | SEQ ID NO: 661 | TKRGQGVPC | | SEQ ID NO: 835 |
| GDFPRGQGF | | SEQ ID NO: 662 | TKRGQGVPH | | SEQ ID NO: 836 |
| GDFPRGQGG | | SEQ ID NO: 663 | TKRGQGVPT | | SEQ ID NO: 837 |
| GDFPRGQGK | | SEQ ID NO: 664 | AGPINTNSA | | SEQ ID NO: 838 |
| GDFPRGQGN | | SEQ ID NO: 665 | HGPINTNSA | | SEQ ID NO: 839 |
| GDFPRGQGQ | | SEQ ID NO: 666 | HGPINTNSL | | SEQ ID NO: 840 |
| GDFPRGQGV | | SEQ ID NO: 667 | LRPINTNSL | | SEQ ID NO: 841 |
| GEFPRGQGA | | SEQ ID NO: 668 | MAPINTNSA | | SEQ ID NO: 842 |
| GEFPRGQGC | | SEQ ID NO: 669 | MGPINTNSA | | SEQ ID NO: 843 |
| GEFPRGQGD | | SEQ ID NO: 670 | QGPINTNSL | | SEQ ID NO: 844 |
| GEFPRGQGE | | SEQ ID NO: 671 | RRPINTNSL | | SEQ ID NO: 845 |
| GEFPRGQGF | | SEQ ID NO: 672 | DGTRRIRGK | | SEQ ID NO: 846 |
| GEFPRGQGG | | SEQ ID NO: 673 | DGTRRIRGQ | | SEQ ID NO: 847 |
| GEFPRGQGI | | SEQ ID NO: 674 | HGTRRIRGK | | SEQ ID NO: 848 |
| GEFPRGQGK | | SEQ ID NO: 675 | HGTRRIRGQ | | SEQ ID NO: 849 |
| GEFPRGQGN | | SEQ ID NO: 676 | IKTRRIRGQ | | SEQ ID NO: 850 |
| GEFPRGQGQ | | SEQ ID NO: 677 | KGTRRIRGQ | | SEQ ID NO: 851 |
| GEFPRGQGV | | SEQ ID NO: 678 | PTTRRIRGK | | SEQ ID NO: 852 |
| GGFPRGQGE | | SEQ ID NO: 679 | PTTRRIRGQ | | SEQ ID NO: 853 |
| GGFPRGQGG | | SEQ ID NO: 680 | PTTRRIRGT | | SEQ ID NO: 854 |
| GGFPRGQGK | | SEQ ID NO: 681 | DKIRGGDGM | | SEQ ID NO: 855 |
| GGFPRGQGN | | SEQ ID NO: 682 | GKIRGGDGM | | SEQ ID NO: 856 |
| GGFPRGQGQ | | SEQ ID NO: 683 | HKIRGGDGM | | SEQ ID NO: 857 |
| GRFPRGQGD | | SEQ ID NO: 684 | QKIRGGDGM | | SEQ ID NO: 858 |
| GRFPRGQGE | | SEQ ID NO: 685 | AHRGGDGKD | | SEQ ID NO: 859 |
| GRFPRGQGQ | | SEQ ID NO: 686 | AHRGGDGKE | | SEQ ID NO: 860 |
| GRFPRGQGV | | SEQ ID NO: 687 | AHRGGDGKF | | SEQ ID NO: 861 |
| GSFPRGQGC | | SEQ ID NO: 688 | AHRGGDGKS | | SEQ ID NO: 862 |
| GSFPRGQGG | | SEQ ID NO: 689 | AKRGGDGKE | | SEQ ID NO: 863 |
| GSFPRGQGQ | | SEQ ID NO: 690 | AKRGGDGKF | | SEQ ID NO: 864 |
| GSFPRGQGV | | SEQ ID NO: 691 | AKRGGDGKK | | SEQ ID NO: 865 |
| HEFPRGQGE | | SEQ ID NO: 692 | AKRGGDGKP | | SEQ ID NO: 866 |
| HEFPRGQGQ | | SEQ ID NO: 693 | AMRGGDGKP | | SEQ ID NO: 867 |
| HQFPRGQGD | | SEQ ID NO: 694 | ATRGGDGKE | | SEQ ID NO: 868 |
| HQFPRGQGE | | SEQ ID NO: 695 | CCRGGDGKP | | SEQ ID NO: 869 |
| HQFPRGQGG | | SEQ ID NO: 696 | CLRGGDGKE | | SEQ ID NO: 870 |
| HQFPRGQGI | | SEQ ID NO: 697 | GKRGGDGKD | | SEQ ID NO: 871 |
| HQFPRGQGK | | SEQ ID NO: 698 | GKRGGDGKE | | SEQ ID NO: 872 |
| IDFPRGQGF | | SEQ ID NO: 699 | GKRGGDGKF | | SEQ ID NO: 873 |
| IDFPRGQGG | | SEQ ID NO: 700 | GKRGGDGKK | | SEQ ID NO: 874 |
| IDFPRGQGQ | | SEQ ID NO: 701 | GKRGGDGKP | | SEQ ID NO: 875 |
| IDFPRGQGV | | SEQ ID NO: 702 | HCRGGDGKP | | SEQ ID NO: 876 |
| KKFPRGQGN | | SEQ ID NO: 703 | HKRGGDGKD | | SEQ ID NO: 877 |
| LKFPRGQGN | | SEQ ID NO: 704 | HKRGGDGKE | | SEQ ID NO: 878 |
| LKFPRGQGQ | | SEQ ID NO: 705 | HKRGGDGKF | | SEQ ID NO: 879 |
| QKFPRGQGD | | SEQ ID NO: 706 | HKRGGDGKP | | SEQ ID NO: 880 |
| QKFPRGQGE | | SEQ ID NO: 707 | HTRGGDGKD | | SEQ ID NO: 881 |
| QKFPRGQGG | | SEQ ID NO: 708 | HTRGGDGKE | | SEQ ID NO: 882 |
| QKFPRGQGN | | SEQ ID NO: 709 | HTRGGDGKF | | SEQ ID NO: 883 |
| QKFPRGQGQ | | SEQ ID NO: 710 | HTRGGDGKK | | SEQ ID NO: 884 |
| QKFPRGQGV | | SEQ ID NO: 711 | HTRGGDGKP | | SEQ ID NO: 885 |
| SSFPRGQGG | | SEQ ID NO: 712 | HTRGGDGKS | | SEQ ID NO: 886 |
| SSFPRGQGQ | | SEQ ID NO: 713 | ICRGGDGKD | | SEQ ID NO: 887 |
| SSFPRGQGV | | SEQ ID NO: 714 | ICRGGDGKE | | SEQ ID NO: 888 |
| SVFPRGQGE | | SEQ ID NO: 715 | ITRGGDGKD | | SEQ ID NO: 889 |
| VDFPRGQGF | | SEQ ID NO: 716 | ITRGGDGKE | | SEQ ID NO: 890 |
| VDFPRGQGG | | SEQ ID NO: 717 | ITRGGDGKF | | SEQ ID NO: 891 |
| VDFPRGQGQ | | SEQ ID NO: 718 | ITRGGDGKK | | SEQ ID NO: 892 |
| VDFPRGQGV | | SEQ ID NO: 719 | ITRGGDGKP | | SEQ ID NO: 893 |
| VEFPRGQGA | | SEQ ID NO: 720 | ITRGGDGKS | | SEQ ID NO: 894 |
| VEFPRGQGD | | SEQ ID NO: 721 | KCRGGDGKP | | SEQ ID NO: 895 |
| VEFPRGQGE | | SEQ ID NO: 722 | KLRGGDGKE | | SEQ ID NO: 896 |
| VEFPRGQGG | | SEQ ID NO: 723 | LHRGGDGKE | | SEQ ID NO: 897 |
| VEFPRGQGI | | SEQ ID NO: 724 | NKRGGDGKD | | SEQ ID NO: 898 |
| VEFPRGQGK | | SEQ ID NO: 725 | NKRGGDGKE | | SEQ ID NO: 899 |
| VEFPRGQGN | | SEQ ID NO: 726 | NKRGGDGKR | | SEQ ID NO: 900 |
| VEFPRGQGQ | | SEQ ID NO: 727 | NLRGGDGKE | | SEQ ID NO: 901 |
| VEFPRGQGR | | SEQ ID NO: 728 | QKRGGDGKP | | SEQ ID NO: 902 |
| VEFPRGQGV | | SEQ ID NO: 729 | SHRGGDGKD | | SEQ ID NO: 903 |
| VGFPRGQGC | | SEQ ID NO: 730 | SHRGGDGKE | | SEQ ID NO: 904 |
| VGFPRGQGD | | SEQ ID NO: 731 | SHRGGDGKF | | SEQ ID NO: 905 |
| VGFPRGQGE | | SEQ ID NO: 732 | SHRGGDGKS | | SEQ ID NO: 906 |
| VGFPRGQGG | | SEQ ID NO: 733 | THRGGDGKD | | SEQ ID NO: 907 |
| VGFPRGQGI | | SEQ ID NO: 734 | THRGGDGKF | | SEQ ID NO: 908 |
| VGFPRGQGK | | SEQ ID NO: 735 | TKRGGDGKD | | SEQ ID NO: 909 |
| VGFPRGQGN | | SEQ ID NO: 736 | TKRGGDGKE | | SEQ ID NO: 910 |
| VGFPRGQGQ | | SEQ ID NO: 737 | TKRGGDGKF | | SEQ ID NO: 911 |
| VSFPRGQGC | | SEQ ID NO: 738 | TKRGGDGKK | | SEQ ID NO: 912 |
| VSFPRGQGG | | SEQ ID NO: 739 | TKRGGDGKP | | SEQ ID NO: 913 |
| WGFPRGQGC | | SEQ ID NO: 740 | TKRGGDGKR | | SEQ ID NO: 914 |
| WGFPRGQGD | | SEQ ID NO: 741 | TTRGGDGKD | | SEQ ID NO: 915 |
| WGFPRGQGE | | SEQ ID NO: 742 | TTRGGDGKE | | SEQ ID NO: 916 |
| WGFPRGQGG | | SEQ ID NO: 743 | TTRGGDGKF | | SEQ ID NO: 917 |
| WGFPRGQGI | | SEQ ID NO: 744 | TTRGGDGKP | | SEQ ID NO: 918 |
| WGFPRGQGQ | | SEQ ID NO: 745 | TTRGGDGKS | | SEQ ID NO: 919 |
| WYFPRGQGD | | SEQ ID NO: 746 | VTRGGDGKD | | SEQ ID NO: 920 |
| WYFPRGQGE | | SEQ ID NO: 747 | VTRGGDGKF | | SEQ ID NO: 921 |
| WYFPRGQGG | | SEQ ID NO: 748 | VTRGGDGKK | | SEQ ID NO: 922 |
| WYFPRGQGI | | SEQ ID NO: 749 | VTRGGDGKP | | SEQ ID NO: 923 |
| WYFPRGQGV | | SEQ ID NO: 750 | VTRGGDGKS | | SEQ ID NO: 924 |
| YEFPRGQGD | | SEQ ID NO: 751 | WCRGGDGKD | | SEQ ID NO: 925 |
| YEFPRGQGE | | SEQ ID NO: 752 | WCRGGDGKE | | SEQ ID NO: 926 |
| YEFPRGQGI | | SEQ ID NO: 753 | WCRGGDGKP | | SEQ ID NO: 927 |
| YEFPRGQGN | | SEQ ID NO: 754 | | | |
| YEFPRGQGQ | | SEQ ID NO: 755 | | | |
| DFPRGQGVE | | SEQ ID NO: 756 | | | |
| DFPRGQGVG | | SEQ ID NO: 757 | | | |
| DFPRGQGVI | | SEQ ID NO: 758 | | | |
| DFPRGQGVM | | SEQ ID NO: 759 | | | |
| DFPRGQGVQ | | SEQ ID NO: 760 | | | |

### Example 1

The present inventors have identified potential receptor binding sites for the Covid-19 virus to the human ACE-2 receptor (Angiotensin-converting enzyme 2 receptor) or other receptor, such as Influenza co-receptor. Due to how easily the virus is transmitted between people other sialylated host cell receptors in the human respiratory tract may be used. A virus neutralising antibody vaccine may be prepared by providing an antibody response with antibodies binding in this or nearby areas of the virus.

The following areas of the Covid-19 virus protein sequence have been identified by the present inventors as potential human ACE-2 receptor or other receptor binding cites includes:

Position 442+6 amino acids of SEQ ID NO:1;

Position 481±6 amino acids of SEQ ID NO:1:
1) The amino acid sequence is non-human-like, indicating that they may be used in a vaccine.
2) This amino acid sequence further has 100% sequence identity with Uniprot ref. D3G2W2 (https://www.uniprot.org/uniprot/D3G2W2)—Neuraminidase (Influenza A virus (A/Nagasaki/07N005/2008(H1N1)));

Position 499±6 amino acids of SEQ ID NO:1:
1) This amino acid sequence further has 100% sequence identity with Uniprot ref. G8EME6 (https://www.uniprot.org/uniprot/G8EME6)—VP1 Protein (Goose Parovirus-PT).
2) The amino acid sequence is non-human like indicating that they may be used in a vaccine.

### Example 2

The 5 peptides used in the vaccine composition were:

(SEQ ID NO: 945)
RRGFKSYGVSPTKLNDSKVGGNYQNRLDSKVGGNY,
(SEQ ID NO: 946)
RRSTPSNGVERRGVEGFNENRFQPTNGRNRGVGYQP,
(SEQ ID NO: 947)
RRGASTEKSNRNGINITRQLLHAPATVRTNGVGYG,
(SEQ ID NO: 948)
RRKSTNLVGGATVTGPGGGVKNKSVGGPLSETK
and
(SEQ ID NO: 950)
RRFYPRGQGVFLEGATNTASWFRSRGFQFPRGQGIG.

They were used to immunize rabbits with the water-in-oil (W/O) adjuvant Montanide ISA-720 VG. The dosing was given intramuscularly (IM).

Forty-eight (48) female New Zealand white rabbits, approximately 3 kg body weight, was separated in eight treatment groups (n=6) with n=3 in each a and b subgroup.

The groups were as follows:

Gr. 1a: 1st dose and 2nd dose: 2.5 mg peptide—0.5 ml injected without adjuvant

Gr. 1b: 1st dose and 2nd dose: 5.0 mg peptide—0.5 ml injected without adjuvant

Gr. 2a: 1st dose 5.0 mg peptide with injection vol=0.5 ml with 70% adjuvant
2nd dose 2.5 mg peptide with injection vol=0.5 ml with 70% adjuvant

Gr. 2b: 1st dose 2.5 mg peptide with injection vol=0.25 ml with 70% adjuvant
2nd dose 1.25 mg (low) peptide with injection vol=0.25 ml—with 70% adjuvant

Gr. 5a: 1st dose 2.5 mg peptide with injection vol=0.5 ml (70% adjuvant)
2nd dose 2.5 mg peptide with injection vol=0.5 ml (70% adjuvant)

Gr. 5b: 1st dose 5.0 mg peptide with injection vol=0.5 ml (70% adjuvant)
2nd dose 5.0 mg peptide with injection vol=0.5 ml (70% adjuvant)

Safety:

The study showed excellent safety.

DTH Test Results:

The DTH test results were recorded 48 hours after ID injection of 100 μl with 500 μg TA (100 μg of each peptide) and measured by multiplying the measured length and width. The results are shown in Table 2.

TABLE 2
Results from DTH test
DTH reaction

| | Week 7 and 48 hour read out | | |
|---|---|---|---|
| Group | Mean | Min | Max |
| 1a | 27.93 | 21.17 | 37.63 |
| 1b | 28.14 | 16.66 | 43.73 |
| 2a | 91.52 | 45.29 | 116.64 |
| 2b | 126.00 | 112.17 | 138.77 |
| 5a | 47.69 | 47.00 | 48.60 |
| 5b | 35.44 | 19.98 | 50.91 |

Antibody Results:

Antibodies to the 5 peptides as well as to the single peptides have been measured. The results are shown in Table 3.

TABLE 3
Results from antibody measurements.

| Group | Week 0* | Week 4* | Week 7* |
|---|---|---|---|
| 1a | 2 | 1 | 12 |
| 1b | 2 | 1 | 12 |
| 2a | 2 | 506 | 42709 |
| 2b | 1 | 341 | 19698 |
| 5a | 2 | 74 | 46918 |
| 5b | 2 | 183 | 59722 |

*Antibody titres in μg/ml

The study showed that the combination of peptides was safe and well tolerated, elicited immune responses in the form of Delayed Type Hypersensitivity (DTH) as well as robust antibody responses.

### Example 3

The present inventors have designed 5 peptides based on amino acid locations from the surface of the Spike trimer in order to secure antibody responses similar to what is provided by the native protein form. See FIG. 1 for details.

Peptides 1 and 4 represent the receptor-binding domain for ACE2 and/or CD209.

Peptide 2 represents the furin-cleavage site and the ganglioside-binding domain.

Peptide 3 represents the ganglioside-binding domain.

Peptide 5 represents the nucleocapsid protein (SEQ ID NO:2).

Peptide 1:
(SEQ ID NO: 951)
rnGVKGFNCYFCLQSYGPTYGVGYQPNNLDSKVGGNYLY [symbol]
RLFRYKGTQGR,
Peptide 2:
(SEQ ID NO: 952)
qtQTN [symbol] SQSIIA [symbol] NLTTRTQKRFANGATW [symbol]
Peptide 3:
(SEQ ID NO: 953)
dCEGKYHKNNKSWCEAVHRSYITPG
Peptide 4:
(SEQ ID NO: 954)
tvRDPQTCDITESNKKFIPL [symbol] QLTPTW [symbol]
Peptide 5:
(SEQ ID NO: 950)
rrFYPRGQGVF [symbol] ATNTASWFR [symbol] FQFPRGQGIG

Single-underlined amino acids are amino acid substitutions

Double-underlined amino acids are amino acid insertions

Amino acids in lower-case letters are D-amino acids.

### REFERENCES

Amrei R. et al., Bioarchives (December) 2020.
Pirone L. et al. Front Mol Biosci. 2020.
Matrosovich et al., 2015
Sorensen B et al. QRB Discovery, Vol. 1, 2020
Sungnak W. et al—ArXiv. Preprint. 2020 Mar. 13
NCBI Reference Sequence: NC_045512.2
GenBank reference QQQ47833.1
Tegally H., et al., Emergence and rapid spread of a new severe acute respiratory syndrome-related coronavirus 2 (SARS-CoV-2) lineage with multiple spike mutations in South AfricamedRxiv preprint doi https address doi.org 10.1101/2020.12.21.20248640, posted Dec. 22, 2020.

1. A monomeric peptide consisting of at least 5, such as at least 6 consecutive amino acids of the sequence of amino acids LDSKVGGNY identified as position 441-449 of SEQ ID NO:1; or a variant thereof containing one, two, or three amino acid substitutions, which variant has not more than one amino acid substitution per three, such as per four, such as per five, such as per six consecutive amino acids of said sequence LDSKVGGNY.

2. The monomeric peptide according to claim 4, which peptide consists of a sequence selected from SEQ ID NO: 33, SEQ ID NO: 34, or SEQ ID NO: 35, or a variant thereof containing at one or two amino acid substitutions, or one amino acid deletion.

3. The monomeric peptide according to any one of claim 4 or 5, which peptide has one or more amino acid substitutions selected from D to E in position 442 of SEQ ID NO:1; S to T in position 443 of SEQ ID NO:1; K to any one of R or homoarginine in position 444 of SEQ ID NO:1; V to any one of L, I, A or norleucine in position 445 of SEQ ID NO:1; N to Q in position 448 of SEQ ID NO:1; or Y to F in position 449 of SEQ ID NO:1.

4. A monomeric peptide consisting of at least 5, such as at least 6 consecutive amino acids of the sequence of amino acids STPCNGVEGFNC identified as position 477-488 of SEQ ID NO:1; or a variant thereof containing one, two, three, or four amino acid substitutions, which variant has not more than one amino acid substitution per three, such as per four, such as per five, such as per six consecutive amino acids of said sequence STPCNGVEGFNC.

5. The monomeric peptide according to claim 1, which peptide consists of a sequence selected from SEQ ID NO: 38, SEQ ID NO: 39, SEQ ID NO: 40, or SEQ ID NO: 41, or a variant thereof containing one or two amino acid substitutions, or one amino acid deletion.

6. The monomeric peptide according to any one of claim 1 or 2, which peptide has one or more amino acid substitutions selected from S to T in position 477 of SEQ ID NO:1; T to S in position 478 of SEQ ID NO:1; C to S in position 480 in SEQ ID NO:1; N to Q in position 481 of SEQ ID NO:1; G to P in position 482 of SEQ ID NO:1; V to any one of L, I, A or norleucin in position 483 of SEQ ID NO:1; E to K or D in position 484 of SEQ ID NO:1; E to D in position 484 of SEQ ID NO:1; F to Y or D in position 486 of SEQ ID NO:1; or N to Q or S in position 487 of SEQ ID NO:1; optionally wherein the amino acid at position 484 in SEQ ID NO:1 is K or E.

7. A monomeric peptide consisting of at least 5, such as at least 6 consecutive amino acids of the sequence of amino acids FQPTNGVGYQP identified as position 497-507 of SEQ ID NO:1; or a variant thereof containing one, two, or three amino acid substitution, which variant has not more than one amino acid substitution per three, such as per four, such as per five, such as per six consecutive amino acids of said sequence FQPTNGVGYQP.

8. The monomeric peptide according to claim 7, which peptide consists of a sequence selected from SEQ ID NO: 42, SEQ ID NO: 43, or SEQ ID NO: 44, or a variant thereof containing one or two amino acid substitutions, or one amino acid deletion.

9. The monomeric peptide according to any one of claim 7 or 8, which peptide has one or more amino acid substitutions selected from F to Y in position 497 of SEQ ID NO:1; Q to N in position 498 of SEQ ID NO:1; T to S in position 500 of SEQ ID NO:1; N to Y or Q in position 501 of SEQ ID NO:1; V to any one of L, I, A or norleucine in position 503 of SEQ ID NO:1; Y to F in position 505 of SEQ ID NO:1; or Q to N in position 506 of SEQ ID NO:1; optionally wherein the amino acid at position 501 in SEQ ID NO:1 is Y or N.

10. A monomeric peptide consisting of at least 5, such as at least 6 consecutive amino acids of the sequence of amino acids FKCYGVSPTKLNDS identified as position 377-391 of SEQ ID NO:1; or a variant thereof containing one, two, or three amino acid substitution, which variant has not more than one amino acid substitution per three, such as per four, such as per five, such as per six consecutive amino acids of said sequence FKCYGVSPTKLNDS.

11. The monomeric peptide according to claim 10, which peptide consists of a sequence selected from SEQ ID NO: 20, SEQ ID NO: 21, SEQ ID NO: 22, SEQ ID NO: 23, SEQ ID NO: 24, or SEQ ID NO: 25, or a variant thereof containing one or two amino acid substitutions, or one amino acid deletion.

12. The monomeric peptide according to any one of claim 10 or 11, which peptide has an amino acid substitution of C to S in position 379 of SEQ ID NO:1.

13. A monomeric peptide consisting of at least 5, such as at least 6 consecutive amino acids of the sequence of amino acids PLSETKCTLKS identified as position 295-305 of SEQ ID NO:1; or a variant thereof containing one, two, or three amino acid substitution, which variant has not more than one amino acid substitution per three, such as per four, such as per five, such as per six consecutive amino acids of said sequence PLSETKCTLKS.

14. The monomeric peptide according to claim 13, which peptide consists of a sequence selected from SEQ ID NO: 109, SEQ ID NO: 110, or SEQ ID NO: 12, or a variant thereof containing one or two amino acid substitutions, or one amino acid deletion.

15. A monomeric peptide consisting of at least 5, such as at least 6 consecutive amino acids of the sequence of amino acids PATVCGPKKSTNLVKNKCV identified as position 521-539 of SEQ ID NO:1; or a variant thereof containing one, two, or three amino acid substitution, which variant has not more than one amino acid substitution per three, such as per four, such as per five, such as per six consecutive amino acids of said sequence PATVCGPKKSTNLVKNKCV.

16. The monomeric peptide according to claim 15, which peptide consists of a sequence selected from SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, or SEQ ID NO: 133, SEQ ID NO: 289, or SEQ ID NO: 290, or a variant thereof containing one or two amino acid substitutions, or one amino acid deletion.

17. The monomeric peptide according to any one of claim 15 or 16, which peptide has one or more amino acid substitutions selected from C to Tin position 525 of SEQ ID NO:1; C to S in position 538 of SEQ ID NO:1; C to S in position 525 of SEQ ID NO:1; and/or C to T in position 538 of SEQ ID NO:1.

18. The monomeric peptide according to any of the above claims, which peptide is at least 5, 6, 7, 8, 9, or 10 amino acids in length, such as 6 or 7 amino acids in length.

19. The monomeric peptide according to any of the above claims, which peptide is not more than 12, 11, 10, 9, 8, 7, 6, or 5 amino acids in length.

20. A monomeric peptide consisting of 6-9 consecutive amino acids of SEQ ID NO:1, which monomeric peptide comprises a sequence of amino acids as defined in any one of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 36, SEQ ID NO: 37, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, or SEQ ID NO: 290; or a variant thereof containing one, two or three amino acid substitutions, or one amino acid deletion.

21. A monomeric peptide consisting of a sequence of amino acids as defined in any one of SEQ ID NO:2 to SEQ ID NO:290, or a variant of thereof containing one or two amino acid substitutions, or one amino acid deletion.

22. A monomeric peptide consisting of 6-9 consecutive amino acids of the sequence of amino acids of SEQ ID NO: 291; or a variant thereof containing one, two, or three amino acid substitutions.

23. A monomeric peptide consisting of 6-9 consecutive amino acids of SEQ ID NO:201, which peptide comprises a sequence selected from SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, SEQ ID NO: 376, SEQ ID NO: 377, SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451, SEQ ID NO: 452, SEQ ID NO: 453, SEQ ID NO: 454, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, SEQ ID NO: 524, SEQ ID NO: 525, SEQ ID NO: 526, SEQ ID NO: 527, SEQ ID NO: 528, SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, SEQ ID NO: 588, SEQ ID NO: 589, SEQ ID NO: 590, SEQ ID NO: 591, SEQ ID NO: 592, SEQ ID NO: 593, SEQ ID NO: 594, SEQ ID NO: 595, SEQ ID NO: 596, SEQ ID NO: 597, SEQ ID NO: 598, SEQ ID NO: 599, SEQ ID NO: 600, SEQ ID NO: 601, SEQ ID NO: 602, SEQ ID NO: 603, SEQ ID NO: 604, SEQ ID NO: 605, SEQ ID NO: 606, SEQ ID NO: 607, SEQ ID NO: 608, SEQ ID NO: 609, SEQ ID NO: 610, SEQ ID NO: 611, SEQ ID NO: 612, SEQ ID NO: 613, SEQ ID NO: 614, SEQ ID NO: 615, SEQ ID NO: 616, SEQ ID NO: 617, SEQ ID NO: 618, SEQ ID NO: 619, SEQ ID NO: 620, SEQ ID NO: 621, SEQ ID NO: 622, SEQ ID NO: 623, SEQ ID NO: 624, SEQ ID NO: 625, SEQ ID NO: 626, SEQ ID NO: 627, SEQ ID NO: 628, SEQ ID NO: 629, SEQ ID NO: 630, SEQ ID NO: 631, SEQ ID NO: 632, SEQ ID NO: 633, SEQ ID NO: 634, SEQ ID NO: 635, SEQ ID NO: 636, SEQ ID NO: 637, SEQ ID NO: 638, SEQ ID NO: 639, SEQ ID NO: 640, SEQ ID NO: 641, SEQ ID NO: 642, SEQ ID NO: 643, SEQ ID NO: 644, SEQ ID NO: 645, SEQ ID NO: 646, SEQ ID NO: 647, SEQ ID NO: 648, SEQ ID NO: 649, SEQ ID NO: 650, SEQ ID NO: 651, SEQ ID NO: 652, SEQ ID NO: 653, SEQ ID NO: 654, SEQ ID NO: 655, SEQ ID NO: 656, SEQ ID NO: 657, SEQ ID NO: 658, SEQ ID NO: 659, SEQ ID NO: 660, SEQ ID NO: 661, SEQ ID NO: 662, SEQ ID NO: 663, SEQ ID NO: 664, SEQ ID NO: 665, SEQ ID NO: 666, SEQ ID NO: 667, SEQ ID NO: 668, SEQ ID NO: 669, SEQ ID NO: 670, SEQ ID NO: 671, SEQ ID NO: 672, SEQ ID NO: 673, SEQ ID NO: 674, SEQ ID NO: 675, SEQ ID NO: 676, SEQ ID NO: 677, SEQ ID NO: 678, SEQ ID NO: 679, SEQ ID NO: 680, SEQ ID NO: 681, SEQ ID NO: 682, SEQ ID NO: 683, SEQ ID NO: 684, SEQ ID NO: 685, SEQ ID NO: 686, SEQ ID NO: 687, SEQ ID NO: 688, SEQ ID NO: 689, SEQ ID NO: 690, SEQ ID NO: 691, SEQ ID NO: 692, SEQ ID NO: 693, SEQ ID NO: 694, SEQ ID NO: 695, SEQ ID NO: 696, SEQ ID NO: 697, SEQ ID NO: 698, SEQ ID NO: 699, SEQ ID NO: 700, SEQ ID NO: 701, SEQ ID NO: 702, SEQ ID NO: 703, SEQ ID NO: 704, SEQ ID NO: 705, SEQ ID NO: 706, SEQ ID NO: 707, SEQ ID NO: 708, SEQ ID NO: 709, SEQ ID NO: 710, SEQ ID NO: 711, SEQ ID NO: 712, SEQ ID NO: 713, SEQ ID NO: 714, SEQ ID NO: 715, SEQ ID NO: 716, SEQ ID NO: 717, SEQ ID NO: 718, SEQ ID NO: 719, SEQ ID NO: 720, SEQ ID NO: 721, SEQ ID NO: 722, SEQ ID NO: 723, SEQ ID NO: 724, SEQ ID NO: 725, SEQ ID NO: 726, SEQ ID NO: 727, SEQ ID NO: 728, SEQ ID NO: 729, SEQ ID NO: 730, SEQ ID NO: 731, SEQ ID NO: 732, SEQ ID NO: 733, SEQ ID NO: 734, SEQ ID NO: 735, SEQ ID NO: 736, SEQ ID NO: 737, SEQ ID NO: 738, SEQ ID NO: 739, SEQ ID NO: 740, SEQ ID NO: 741, SEQ ID NO: 742, SEQ ID NO: 743, SEQ ID NO: 744, SEQ ID NO: 745, SEQ ID NO: 746, SEQ ID NO: 747, SEQ ID NO: 748, SEQ ID NO: 749, SEQ ID NO: 750, SEQ ID NO: 751, SEQ ID NO: 752, SEQ ID NO: 753, SEQ ID NO: 754, SEQ ID NO: 755, SEQ ID NO: 756, SEQ ID NO: 757, SEQ ID NO: 758, SEQ ID NO: 759, SEQ ID NO: 760, SEQ ID NO: 761, SEQ ID NO: 762, SEQ ID NO: 763, SEQ ID NO: 764, SEQ ID NO: 765, SEQ ID NO: 766, SEQ ID NO: 767, SEQ ID NO: 768, SEQ ID NO: 769, SEQ ID NO: 770, SEQ ID NO: 771, SEQ ID NO: 772, SEQ ID NO: 773, SEQ ID NO: 774, SEQ ID NO: 775, SEQ ID NO: 776, SEQ ID NO: 777, SEQ ID NO: 778, SEQ ID NO: 779, SEQ ID NO: 780, SEQ ID NO: 781, SEQ ID NO: 782, SEQ ID NO: 783, SEQ ID NO: 784, SEQ ID NO: 785, SEQ ID NO: 786, SEQ ID NO: 787, SEQ ID NO: 788, SEQ ID NO: 789, SEQ ID NO: 790, SEQ ID NO: 791, SEQ ID NO: 792, SEQ ID NO: 793, SEQ ID NO: 794, SEQ ID NO: 795, SEQ ID NO: 796, SEQ ID NO: 797, SEQ ID NO: 798, SEQ ID NO: 799, SEQ ID NO: 800, SEQ ID NO: 801, SEQ ID NO: 802, SEQ ID NO: 803, SEQ ID NO: 804, SEQ ID NO: 805, SEQ ID NO: 806, SEQ ID NO: 807, SEQ ID NO: 808, SEQ ID NO: 809, SEQ ID NO: 810, SEQ ID NO: 811, SEQ ID NO: 812, SEQ ID NO: 813, SEQ ID NO: 814, SEQ ID NO: 815, SEQ ID NO: 816, SEQ ID NO: 817, SEQ ID NO: 818, SEQ ID NO: 819, SEQ ID NO: 820, SEQ ID NO: 821, SEQ ID NO: 822, SEQ ID NO: 823, SEQ ID NO: 824, SEQ ID NO: 825, SEQ ID NO: 826, SEQ ID NO: 827, SEQ ID NO: 828, SEQ ID NO: 829, SEQ ID NO: 830, SEQ ID NO: 831, SEQ ID NO: 832, SEQ ID NO: 833, SEQ ID NO: 834, SEQ ID NO: 835, SEQ ID NO: 836, SEQ ID NO: 837, SEQ ID NO: 838, SEQ ID NO: 839, SEQ ID NO: 840, SEQ ID NO: 841, SEQ ID NO: 842, SEQ ID NO: 843, SEQ ID NO: 844, SEQ ID NO: 845, SEQ ID NO: 846, SEQ ID NO: 847, SEQ ID NO: 848, SEQ ID NO: 849, SEQ ID NO: 850, SEQ ID NO: 851, SEQ ID NO: 852, SEQ ID NO: 853, SEQ ID NO: 854, SEQ ID NO: 855, SEQ ID NO: 856, SEQ ID NO: 857, SEQ ID NO: 858, SEQ ID NO: 859, SEQ ID NO: 860, SEQ ID NO: 861, SEQ ID NO: 862, SEQ ID NO: 863, SEQ ID NO: 864, SEQ ID NO: 865, SEQ ID NO: 866, SEQ ID NO: 867, SEQ ID NO: 868, SEQ ID NO: 869, SEQ ID NO: 870, SEQ ID NO: 871, SEQ ID NO: 872, SEQ ID NO: 873, SEQ ID NO: 874, SEQ ID NO: 875, SEQ ID NO: 876, SEQ ID NO: 877, SEQ ID NO: 878, SEQ ID NO: 879, SEQ ID NO: 880, SEQ ID NO: 881, SEQ ID NO: 882, SEQ ID NO: 883, SEQ ID NO: 884, SEQ ID NO: 885, SEQ ID NO: 886, SEQ ID NO: 887, SEQ ID NO: 888, SEQ ID NO: 889, SEQ ID NO: 890, SEQ ID NO: 891, SEQ ID NO: 892, SEQ ID NO: 893, SEQ ID NO: 894, SEQ ID NO: 895, SEQ ID NO: 896, SEQ ID NO: 897, SEQ ID NO: 898, SEQ ID NO: 899, SEQ ID NO: 900, SEQ ID NO: 901, SEQ ID NO: 902, SEQ ID NO: 903, SEQ ID NO: 904, SEQ ID NO: 905, SEQ ID NO: 906, SEQ ID NO: 907, SEQ ID NO: 908, SEQ ID NO: 909, SEQ ID NO: 910, SEQ ID NO: 911, SEQ ID NO: 912, SEQ ID NO: 913, SEQ ID NO: 914, SEQ ID NO: 915, SEQ ID NO: 916, SEQ ID NO: 917, SEQ ID NO: 918, SEQ ID NO: 919, SEQ ID NO: 920, SEQ ID NO: 921, SEQ ID NO: 922, SEQ ID NO: 923, SEQ ID NO: 924, SEQ ID NO: 925, SEQ ID NO: 926, or SEQ ID NO: 927, or a variant thereof containing one or two amino acid substitutions, or one amino acid deletion.

24. A monomeric peptide consisting of a sequence of amino acids as defined in any one of SEQ ID NO:292 to SEQ ID NO:927, or a variant thereof containing one or two amino acid substitutions or one amino acid deletion.

25. The monomeric peptide according to any one of the preceding claims, wherein the overall net charge of said peptide is equal to or above 0, such as above 1, 2, 3, 4, or 5.

26. The monomeric peptide according to any one of the preceding claims, wherein said monomeric peptide is capable of inducing a humoral immune response.

27. The monomeric peptide according to any one of the preceding claims, wherein said monomeric peptide comprises at least one amino acid selected from a Cys, a Lys, an Asp, and a Glu residue, or derivatives thereof.

28. The monomeric peptide according to any one of the preceding claims, which monomeric peptide has delayed proteolytic degradation in the N-terminus, such as by incorporation of the first 1, 2, or 3 amino acids in the N-terminus in the D-form, or by incorporation of the first 1, 2, or 3 amino acids in the N-terminus in beta or gamma form.

29. A monomeric polypeptide from 10 to 80 amino acids in length, which polypeptide comprises or consist of two, three or four consecutive sequences of amino acids as independently defined in any one of claims 4-6 and 17-28, which two, three or four consecutive sequences of amino acids is optionally separated by, or having in the N- or C-terminal of the polypeptide, a sequence of amino acids selected from G, R, GG, GR, RG, RR, RRR, GGG, GGR, RGG, GRG, RRG, GRR, or RGR.

30. The polypeptide according to claim 29, which polypeptide consists of the sequence of amino acids RGPCNGVEGRGTPCNGVGRGGVEGFN.

31. A monomeric polypeptide from 10 to 80 amino acids in length, which polypeptide comprises or consist of two, three or four consecutive sequences of amino acids independently as defined in any one of claims 1-3 and 17-28, which two, three or four consecutive sequences of amino acids is optionally separated by, or having in the N- or C-terminal of the polypeptide, a sequence of amino acids selected from G, R, GG, GR, RG, RR, RRR, GGG, GGR, RGG, GRG, RRG, GRR, or RGR.

32. The polypeptide according to claim 31, which polypeptide consists of the sequence of amino acids RGKVGGNYGRGDSKVGGRG.

33. A monomeric polypeptide from 10 to 80 amino acids in length, which polypeptide comprises or consist of two, three or four consecutive sequences of amino acids independently as defined in any one of claims 7-9 and 17-28, which two, three or four consecutive sequences of amino acids is optionally separated by, or having in the N- or C-terminal of the polypeptide, a sequence of amino acids selected from G, R, GG, GR, RG, RR, RRR, GGG, GGR, RGG, GRG, RRG, GRR, or RGR.

34. The polypeptide according to claim 33, which polypeptide consists of the sequence of amino acids RGTNGVGYGRGFQPTNGGRGGVGYQP.

35. A monomeric polypeptide from 10 to 80 amino acids in length, which polypeptide comprises or consist of two, three or four consecutive sequences of amino acids independently as defined in any one of claims 10-12 and 17-28, which two, three or four consecutive sequences of amino acids is optionally separated by, or having in the N- or C-terminus of the polypeptide, a sequence of amino acids selected from G, R, GG, GR, RG, RR, RRR, GGG, GGR, RGG, GRG, RRG, GRR, or RGR.

36. The polypeptide according to claim 35, which polypeptide consists of the sequence of amino acids RGCYGVSPGRGFKCYGVGRGVSPTKL.

37. A monomeric polypeptide from 10 to 80 amino acids in length, which polypeptide comprises or consist of two, three or four consecutive sequences of amino acids independently as defined in any one of claims 13-14 and 17-28, which two, three or four consecutive sequences of amino acids is optionally separated by, or having in the N- or C-terminus of the polypeptide, a sequence of amino acids selected from G, R, GG, GR, RG, RR, RRR, GGG, GGR, RGG, GRG, RRG, GRR, or RGR.

38. The polypeptide according to claim 37, which polypeptide consists of the sequence of amino acids RGPLSETKGRGKCTLKSGRGSETKCT.

39. A monomeric polypeptide from 10 to 80 amino acids in length, which polypeptide comprises or consist of two, three or four consecutive sequences of amino acids independently as defined in any one of claims 15-28, which two, three or four consecutive sequences of amino acids is optionally separated by, or having in the N- or C-terminus of the polypeptide, a sequence of amino acids selected from G, R, GG, GR, RG, RR, RRR, GGG, GGR, RGG, GRG, RRG, GRR, or RGR.

40. The polypeptide according to claim 39, which polypeptide consists of the sequence of amino acids RGTVCGPKGRGPKKSTNGRGVKNKCV.

41. A monomeric polypeptide from 10 to 80 amino acids in length, which polypeptide comprises or consist of two, three or four consecutive sequences of amino acids independently as defined in any one of claims 1-28, which two, three or four consecutive sequences of amino acids is optionally separated by or having in the N- or C-terminal of the polypeptide a sequence of amino acids selected from G, R, GG, GR, RG, RR, RRR, GGG, GGR, RGG, GRG, RRG, GRR, or RGR.

42. A monomeric polypeptide of 10 to 80 amino acids in length, which monomeric polypeptide comprises
(a) a first monomeric peptide according to any one of claims 4 to 6,
(b) a second monomeric peptide according to any one of claims 7 to 9, and
(c) optionally, a third monomeric peptide according to any one of claims 1 to 3,
wherein the N- or C-terminal of the monomeric polypeptide has a sequence of amino acids selected from G, R, GG, GR, RG, RR, RRR, GGG, GGR, RGG, GRG, RRG, GRR, and RGR.

43. The monomeric polypeptide according to claim 42, wherein
(a) the first monomeric peptide comprises the sequence of amino acids NGVKGFNC identified as position 481-488 of SEQ ID NO:1 with an E484K amino acid substitution, or the sequence of amino acids STPSNGVE identified as position 477-493 with a C480S amino acid substitution;
(b) the second monomeric peptide comprises the sequence of amino acids GVGYQP (SEQ ID NO:44); and
(c) the third monomeric peptide comprises the sequence of amino acids KVGGNY (SEQ ID NO:35).

44. The monomeric polypeptide according to any one of claim 42 or 43, wherein the third monomeric polypeptide comprises the sequence of amino acids LDSKVGGNY identified as position 441-449 of SEQ ID NO:1.

45. A polypeptide comprising or consisting of the sequence of amino acids RNGVKGFNCYFCLQSYGPTYGVGYQPNNLDSKVGGNYLYCRLFRYKGTQGR (SEQ ID NO:951), or a variant thereof comprising one, two, three, or four amino acid substitutions, insertions or deletions, which variant has no more than one amino acid substitution, insertion or deletion per three, such as per four, such as per five, such as per six consecutive amino acids of SEQ ID NO:951.

46. A monomeric polypeptide of 10 to 80 amino acids in length, which monomeric polypeptide comprises
(a) a first monomeric peptide comprising the sequence of amino acids FYPRGQGVF (SEQ ID NO:292),
(b) a second monomeric peptide comprising the sequence of amino acids ATNTASWFR (SEQ ID NO:293),
(c) a third monomeric peptide comprising the sequence of amino acids FQFPRGQGI (SEQ ID NO:294), or
(d) a combination of (a) and (b), (a) and (c), (b) and (c), or (a) to (c).

47. A polypeptide comprising or consisting of a sequence of amino acids RRFYPRGQGVFLEGATNTASWFRSRGFQFPRGQGIG (SEQ ID NO:950), or a variant thereof comprising one, two, three, or four amino acid substitutions, insertions or deletions, which variant has no more than one amino acid substitution, insertion or deletion per three, such as per four, such as per five, such as per six consecutive amino acids of SEQ ID NO:950.

48. A polypeptide comprising or consisting of a sequence of amino acids QTQTNGSQSIIAGCGNLTTRTQKRFANGATWC (SEQ ID NO:952), or a variant thereof comprising one, two, three, or four amino acid substitutions, insertions or deletions, which variant has no more than one amino acid substitution, insertion or deletion per three, such as per four, such as per five, such as per six consecutive amino acids of the amino acid sequence of said SEQ ID NO.

49. A polypeptide comprising or consisting of a sequence of amino acids selected from DCEGKYHKNNKSWCEAVHRSYITPG (SEQ ID NO:953), or a variant thereof comprising one, two, three, or four amino acid substitutions, insertions or deletions, which variant has no more than one amino acid substitution, insertion or deletion per three, such as per four, such as per five, such as per six consecutive amino acids of the amino acid sequence of said SEQ ID NO.

50. A polypeptide comprising or consisting of a sequence of amino acids selected from TVRDPQTCDITESNKKFIPLGCGQLTPTWGRR (SEQ ID NO:954), or a variant thereof comprising one, two, three, or four amino acid substitutions, insertions or deletions, which variant has no more than one amino acid substitution, insertion or deletion per three, such as per four, such as per five, such as per six consecutive amino acids of the amino acid sequence of said SEQ ID NO.

51. The monomeric polypeptide according to any one of claims 29-50, which is a cyclic polypeptide.

52. The monomeric polypeptide according to any one of claims 29 to 51, which comprises at least one intrachain bond, such as a disulphide bond.

53. The monomeric polypeptide according to any one of claims 29-52, which monomeric polypeptide is of 10-80 amino acids, such as of 11-80 amino acids, such as of 12-80 amino acids, such as of 13-80 amino acids, such as of 14-80 amino acids, such as of 15-80 amino acids, such as of 16-80 amino acids, such as of 17-80 amino acids, such as of 18-80 amino acids, such as of 19-80 amino acids, such as of 20-80 amino acids, such as of 21-80 amino acids, such as of 22-80 amino acids, such as of 23-80 amino acids, such as of 24-80 amino acids, such as of 25-80 amino acids, such as of 26-80 amino acids, such as of 27-80 amino acids, such as of 28-80 amino acids, such as of 29-80 amino acids, such as of 30-80 amino acids, such as of 31-80 amino acids, such as of 32-80 amino acids, such as of 33-80 amino acids, such as of 34-80 amino acids, such as of 35-80 amino acids, such as of 36-80 amino acids, such as of 37-80 amino acids, such as of 38-80 amino acids, such as of 39-80 amino acids, such as of 40-80 amino acids, such as of 42-80 amino acids, such as of 44-80 amino acids, such as of 46-80 amino acids, such as of 48-80 amino acids, such as of 50-80 amino acids, such as of 52-80 amino acids, such as of 54-80 amino acids, such as of 56-80 amino acids, such as of 58-80 amino acids, such as of 59-80 amino acids, such as of 60-80 amino acids, such as of 61-80 amino acids, such as of 62-80 amino acids, such as of 63-80 amino acids, such as of 64-80 amino acids, such as of 65-80 amino acids, such as of 66-80 amino acids, such as of 67-80 amino acids, such as of 68-80 amino acids, such as of 69-80 amino acids, such as of 70-80 amino acids in length.

54. The monomeric polypeptide according to any one of claims 29-53, which monomeric polypeptide is of 10-78 amino acids, such as 10-76 amino acids, such as 10-74 amino acids, such as 10-72 amino acids, such as 10-70 amino acids, such as 10-68 amino acids, such as 10-66 amino acids, such as 10-64 amino acids, such as 10-62 amino acids, such as 10-60 amino acids, such as 10-58 amino acids, such as 10-56 amino acids, such as 10-54 amino acids, such as 10-52 amino acids, such as 10-50 amino acids, such as 10-48 amino acids, such as 10-46 amino acids, such as 10-44 amino acids, such as 10-42 amino acids, such as 10-40 amino acids, such as 10-39 amino acids, such as 10-38 amino acids, such as 10-37 amino acids, such as 10-36 amino acids, such as 10-35 amino acids, such as 10-34 amino acids, such as 10-33 amino acids, such as 10-32 amino acids, such as 10-31 amino acids, such as 10-30 amino acids, such as 10-29 amino acids, such as 10-28 amino acids, such as 10-27 amino acids, such as 10-26 amino acids, such as 10-25 amino acids, such as 10-24 amino acids, such as 10-23 amino acids, such as 10-22 amino acids, such as 10-21 amino acids, such as 10-20 amino acids, such as 10-19 amino acids, such as 10-18 amino acids, such as 10-17 amino acids, such as 10-16 amino acids, such as 10-15 amino acids, such as 10-14 amino acids, such as 10-13 amino acids, such as 10-12 amino acids, such as 10-11 amino acids in length.

55. The monomeric polypeptide according to any one of claims 29-54, which monomeric polypeptide consist of not more than about 70 amino acids, such as not more than about 65 amino acids, such as not more than about 60 amino acids, such as not more than about 55 amino acids, such as not more than about 50 amino acids, such as not more than about 45 amino acids, such as not more than about 40 amino acids, such as not more than about 38 amino acids, such as not more than about 36 amino acids, such as not more than about 34 amino acids, such as not more than about 32 amino acids, such as not more than about 30 amino acids, such as not more than about 28 amino acids, such as not more than about 26 amino acids, such as not more than about 24 amino acids, such as not more than about 22 amino acids, such as not more than about 20 amino acids, such as not more than about 18 amino acids, such as not more than about 16 amino acids, such as not more than about 14 amino acids, such as not more than about 12 amino acids, such as not more than about 10 amino acids in length.

56. The monomeric polypeptide according to any one of claims 29-54, which monomeric polypeptide consist of at least about 10 amino acids, such as at least about 12 amino acids, such as at least about 14 amino acids, such as at least about 16 amino acids, such as at least about 18 amino acids, such as at least about 20 amino acids, such as at least about 22 amino acids, such as at least about 24 amino acids, such as at least about 26 amino acids, such as at least about 28 amino acids, such as at least about 30 amino acids, such as at least about 32 amino acids, such as at least about 34 amino acids, such as at least about 36 amino acids, such as at least about 38 amino acids, such as at least about 40 amino acids, such as at least about 45 amino acids, such as at least about 50 amino acids, such as at least about 55 amino acids, such as at least about 60 amino acids, such as at least about 65 amino acids, such as at least about 70 amino acids, such as at least about 75 amino acids in length.

57. The monomeric polypeptide according to any one of claims 29-56, wherein the overall net charge of said polypeptide is equal to or above 0, such as above 1, 2, 3, 4, or 5.

58. The monomeric polypeptide according to any one of claims 29-57, wherein said monomeric polypeptide is capable of inducing a humoral immune response.

59. A monomeric polypeptide comprising or consisting of a sequence of amino acids selected from RGPCNGVEGRGTPCNGVGRGGVEGFN (SEQ ID NO:934), RGKVGGNYGRGDSKVGGRG (SEQ ID NO:935), RGTNGVGYGRGFQPTNGGRGGVGYQP (SEQ ID NO:936), RGCYGVSPGRGFKCYGVGRGVSPTKL (SEQ ID NO:937), RGPLSETKGRGKCTLKSGRGSETKCT (SEQ ID NO:938), RGTVCGPKGRGPKKSTNGRGVKNKCV (SEQ ID NO:939), RGKVGGNYQNRLDSKVGGRN (SEQ ID NO:940), RRGPCNGVENRTPSNGVENRNGVEGFNNRSTPSNG (SEQ ID NO:941), RRRGSTPCNGVEGFQSNGVEGFNCWQRR (SEQ ID NO:942), RGTNGVGYNNRFQPTNGRNRGVGYQPRN (SEQ ID NO:943), RGASTEKSNRNGINITRQRRLLHAPATVG (SEQ ID NO:944), RRGFKSYGVSPTKLNDSKVGGNYQNRLDSKVGGNY (SEQ ID NO:945), RRSTPSNGVERRGVEGFNENRFQPTNGRNRGVGYQP (SEQ ID NO:946), RRGASTEKSNRNGINITRQLLHAPATVRTNGVGYG (SEQ ID NO:947), RRKSTNLVGGATVTGPGGGVKNKSVGGPLSETK (SEQ ID NO:948), and RRKSTNLVGGQLTPTWGGGVKNKSVGGPLSETK (SEQ ID NO:949), or a variant of any thereof, comprising one, two, three, or four amino acid substitutions, insertions or deletions, which variant has no more than one amino acid substitution, insertion or deletion per three, such as per four, such as per five, such as per six consecutive amino acids of the amino acid sequence of said SEQ ID NO.

60. A multimeric peptide, such as a dimeric peptide comprising at least a first monomeric peptide or polypeptide as defined in any one of claims 1-59, covalently joined to at least a second monomeric peptide or polypeptide independently as defined in any one of claims 1-59, the monomeric polypeptides being covalently joined, such as joined by a disulfide (S—S) bond between a Cys residue in each monomeric peptide.

61. The multimeric, such as dimeric peptide according to claim 60, wherein said first and said second monomeric peptides or polypeptides are identical in sequence.

62. The multimeric, such as dimeric peptide according to claim 60, wherein said first and said second monomeric peptides or polypeptides are different in sequence.

63. A conjugate or fusion protein comprising a monomeric peptide as defined in any one of claims 1 to 28, a monomeric polypeptide as defined in any one of claims 29 to 59, or a multimeric polypeptide as defined in any one of claims 60 to 62, and a second moiety, such as a polymer or carrier molecule.

64. A combination comprising
(a) a first monomeric peptide as defined in any one of claims 1 to 28 and a second monomeric peptide as defined in any one of claims 1 to 28,
(b) a first monomeric polypeptide as defined in any one of claims 29 to 59 and a second monomeric polypeptide as defined in any one of claims 29 to 59;
(c) the polypeptides of claims 45, 47, 48, 49 and 50; or
(d) the polypeptides of SEQ ID NOS:945, 946, 947, 948 and 950.

65. A nucleic acid encoding at least one monomeric peptide or polypeptide as defined in any one of claims 1-59, or multimeric, such as dimeric, peptide as defined in any one of claims 60 to 62, or conjugate or fusion protein as defined in claim 63, or combination as defined in claim 64.

66. A vector comprising a polynucleotide sequence comprising a nucleic acid as defined in claim 65.

67. A pharmaceutical composition comprising a monomeric peptide or polypeptide as defined in any one of claims 1-59, or multimeric, such as dimeric, peptide as defined in any one of claims 60-62, or conjugate or fusion protein as defined in claim 63, or combination as defined in claim 64, or nucleic acid as defined in claim 65, or vector as defined in claim 66, optionally further comprising a pharmaceutically acceptable diluent or vehicle and optionally an immunological adjuvant, such as IMM-101.

68. A pharmaceutical composition comprising a monomeric peptide or polypeptide as defined in any one of claims 1-59, or multimeric, such as dimeric, peptide as defined in any one of claims 60-62, or conjugate or fusion protein as defined in claim 63, or combination as defined in claim 63, formulated in a peptide slow-release formulation.

69. The pharmaceutical composition according to any one of claims 67 and 68, which composition comprises a low viscosity, non-liquid crystalline, mixture of:
a) 25-55 wt. % of at least one diacyl glycerol and/or at least one tocopherol;
b) 25-55 wt. % of at least one phospholipid component comprising phospholipids having
i) polar head groups comprising more than 50% phosphatidyl ethanolamine, and
ii) two acyl chains each independently having 16 to 20 carbons wherein at least one acyl chain has at least one unsaturation in the carbon chain, and there are no more than four unsaturations over two carbon chains;
c) 5-25 wt. % of at least one biocompatible, oxygen containing, low viscosity organic solvent;
wherein 0.1-10 wt. % of at least one monomeric peptide, monomeric polypeptide, multimeric peptide, conjugate, fusion protein or combination, is dissolved or dispersed in the low viscosity mixture;
and wherein the pre-formulation forms, or is capable of forming, at least one non-lamellar liquid crystalline phase structure upon contact with an aqueous fluid.

70. Use of a monomeric peptide or polypeptide as defined in any one of claims 1-59, or a multimeric, such as dimeric, peptide as defined in any one of claims 60-62, or conjugate or fusion protein as defined in claim 63, or combination as defined in claim 64, or nucleic acid as defined in claim 65, or vector as defined in claim 66, or a pharmaceutical composition according to any one of claims 67-69 as a pharmaceutical, such as a vaccine.

71. A method for reducing and/or delaying pathological effects of an infection with corona virus, such as with human Wuhan seafood market pneumonia virus isolate Wuhan-Hu-1, in a human infected with such virus, the method comprising administering an effective amount of a monomeric peptide or polypeptide as defined in any one of claims 1-59, or multimeric, such as dimeric, peptide as defined in any one of claims 60-62, or conjugate or fusion protein as defined in claim 63, or combination as defined in claim 64, or nucleic acid as defined in claim 65, or vector as defined in claim 66, or a pharmaceutical composition according to any one of claims 67-69.

72. A method of inducing immunity in an animal, comprising administering at least once an immunogenically effective amount of a monomeric peptide or polypeptide as defined in any one of claims 1-59, or multimeric peptide as defined in any one of claims 60-62, or conjugate or fusion protein as defined in claim 63, or combination as defined in claim 64, or nucleic acid as defined in claim 65, or vector as defined in claim 66, or a pharmaceutical composition according to any one of claims 67-69, so as to induce immunity against corona virus, such as against human Wuhan seafood market pneumonia virus isolate Wuhan-Hu-1, in the animal.

73. A method for inducing a therapeutic or ameliorating immune response against corona virus, such as with human Wuhan seafood market pneumonia virus isolate Wuhan-Hu-1, the method comprising administering an effective amount of a monomeric peptide or polypeptide as defined in any one of claims 1-59, or multimeric, such as dimeric, peptide as defined in any one of claims 60-62, or conjugate or fusion protein as defined in claim 63, or combination as defined in claim 64, or nucleic acid as defined in claim 65, or vector as defined in claim 66, or a pharmaceutical composition according to any one of claims 67-69, or a pharmaceutical composition according to any one or claims 66-67.

74. Use of a monomeric peptide or polypeptide as defined in any one of claims 1-59, or multimeric, such as dimeric, peptide as defined in any one of claims 60-62, or conjugate or fusion protein as defined in claim 63, or combination as defined in claim 64, or nucleic acid as defined in claim 65, or vector as defined in claim 66, or a pharmaceutical composition according to any one of claims 67-69, for diagnostic use.

75. Use of a monomeric peptide or polypeptide as defined in any one of claims 1-59, or multimeric, such as dimeric, peptide as defined in any one of claims 60-62; or conjugate or fusion protein as defined in claim 63, or combination as defined in claim 64, or nucleic acid as defined in claim 65, or vector as defined in claim 66, or a pharmaceutical composition according to any one of claims 67-69, in the characterization of corona virus, such as with human Wuhan seafood market pneumonia virus isolate Wuhan-Hu-1 in vitro.


SEQUENCE LISTING (960 sequences)

SEQ ID NO: 1 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 6 aa, Artificial Sequence
  Non-human like sequence
ASTEKS

SEQ ID NO: 3 - protein, 6 aa, Artificial Sequence
  Non-human like sequence
STEKSN

SEQ ID NO: 4 - protein, 6 aa, Artificial Sequence
  Non-human like sequence
LGVYYH

SEQ ID NO: 5 - protein, 6 aa, Artificial Sequence
  Non-human like sequence
GVYYHK

SEQ ID NO: 6 - protein, 6 aa, Artificial Sequence
  Non-human like sequence
VYSSAN

SEQ ID NO: 7 - protein, 6 aa, Artificial Sequence
  Non-human like sequence
SANNCT

SEQ ID NO: 8 - protein, 6 aa, Artificial Sequence
  Non-human like sequence
YVSQPF

SEQ ID NO: 9 - protein, 6 aa, Artificial Sequence
  Non-human like sequence
LEGKQG

SEQ ID NO: 10 - protein, 6 aa, Artificial Sequence
  Non-human like sequence
GKQGNF

SEQ ID NO: 11 - protein, 6 aa, Artificial Sequence
  Non-human like sequence
GINITR

SEQ ID NO: 12 - protein, 6 aa, Artificial Sequence
  Non-human like sequence
KCTLKS

SEQ ID NO: 13 - protein, 6 aa, Artificial Sequence
  Non-human like sequence
TESIVR

SEQ ID NO: 14 - protein, 6 aa, Artificial Sequence
  Non-human like sequence
ITNLCP

SEQ ID NO: 15 - protein, 6 aa, Artificial Sequence
  Non-human like sequence
LCPFGE

SEQ ID NO: 16 - protein, 6 aa, Artificial Sequence
  Non-human like sequence
ATRFAS

SEQ ID NO: 17 - protein, 6 aa, Artificial Sequence
  Non-human like sequence
TRFASV

SEQ ID NO: 18 - protein, 6 aa, Artificial Sequence
  Non-human like sequence
SNCVAD

SEQ ID NO: 19 - protein, 6 aa, Artificial Sequence
  Non-human like sequence
VLYNSA

SEQ ID NO: 20 - protein, 6 aa, Artificial Sequence
  Non-human like sequence
FKCYGV

SEQ ID NO: 21 - protein, 6 aa, Artificial Sequence
  Non-human like sequence
KCYGVS

SEQ ID NO: 22 - protein, 6 aa, Artificial Sequence
  Non-human like sequence
CYGVSP

SEQ ID NO: 23 - protein, 6 aa, Artificial Sequence
  Non-human like sequence
YGVSPT

SEQ ID NO: 24 - protein, 6 aa, Artificial Sequence
  Non-human like sequence
GVSPTK

SEQ ID NO: 25 - protein, 6 aa, Artificial Sequence
  Non-human like sequence
VSPTKL

SEQ ID NO: 26 - protein, 6 aa, Artificial Sequence
  Non-human like sequence
ADSFVI

SEQ ID NO: 27 - protein, 6 aa, Artificial Sequence
  Non-human like sequence
QIAPGQ

SEQ ID NO: 28 - protein, 6 aa, Artificial Sequence
  Non-human like sequence
TGKIAD

SEQ ID NO: 29 - protein, 6 aa, Artificial Sequence
  Non-human like sequence
GKIADY

SEQ ID NO: 30 - protein, 6 aa, Artificial Sequence
  Non-human like sequence
KIADYN

SEQ ID NO: 31 - protein, 6 aa, Artificial Sequence
  Non-human like sequence
DDFTGC

SEQ ID NO: 32 - protein, 6 aa, Artificial Sequence
  Non-human like sequence
FTGCVI

SEQ ID NO: 33 - protein, 6 aa, Artificial Sequence
  Non-human like sequence
LDSKVG

SEQ ID NO: 34 - protein, 6 aa, Artificial Sequence
  Non-human like sequence
DSKVGG

SEQ ID NO: 35 - protein, 6 aa, Artificial Sequence
  Non-human like sequence
KVGGNY

SEQ ID NO: 36 - protein, 6 aa, Artificial Sequence
  Non-human like sequence
KSNLKP

SEQ ID NO: 37 - protein, 6 aa, Artificial Sequence
  Non-human like sequence
EIYQAG

SEQ ID NO: 38 - protein, 6 aa, Artificial Sequence
  Non-human like sequence
STPCNG

SEQ ID NO: 39 - protein, 6 aa, Artificial Sequence
  Non-human like sequence
TPCNGV

SEQ ID NO: 40 - protein, 6 aa, Artificial Sequence
  Non-human like sequence
PCNGVE

SEQ ID NO: 41 - protein, 6 aa, Artificial Sequence
  Non-human like sequence
GVEGFN

SEQ ID NO: 42 - protein, 6 aa, Artificial Sequence
  Non-human like sequence
FQPTNG

SEQ ID NO: 43 - protein, 6 aa, Artificial Sequence
  Non-human like sequence
TNGVGY

SEQ ID NO: 44 - protein, 6 aa, Artificial Sequence
  Non-human like sequence
GVGYQP

SEQ ID NO: 45 - protein, 6 aa, Artificial Sequence
  Non-human like sequence
LLHAPA

SEQ ID NO: 46 - protein, 6 aa, Artificial Sequence
  Non-human like sequence
LHAPAT

SEQ ID NO: 47 - protein, 6 aa, Artificial Sequence
  Non-human like sequence
HAPATV

SEQ ID NO: 48 - protein, 12 aa, Artificial Sequence
  Non-human like sequence
STPCNGVEGFSC

SEQ ID NO: 49 - protein, 12 aa, Artificial Sequence
  Non-human like sequence
STPCNGVEGFNC

SEQ ID NO: 50 - protein, 12 aa, Artificial Sequence
  Non-human like sequence
STPCNGVEGFQC

SEQ ID NO: 51 - protein, 12 aa, Artificial Sequence
  Non-human like sequence
PCNPADFNCYFP

SEQ ID NO: 52 - protein, 12 aa, Artificial Sequence
  Non-human like sequence
PCQGLEFNCYWP

SEQ ID NO: 53 - protein, 12 aa, Artificial Sequence
  Non-human like sequence
PCQGLEFQCYWP

SEQ ID NO: 54 - protein, 12 aa, Artificial Sequence
  Non-human like sequence
PCQGLEFSCYWP

SEQ ID NO: 55 - protein, 6 aa, Artificial Sequence
  Non-human like sequence
STPSNG

SEQ ID NO: 56 - protein, 6 aa, Artificial Sequence
  Non-human like sequence
VEGFSC

SEQ ID NO: 57 - protein, 6 aa, Artificial Sequence
  Non-human like sequence
VEGFNC

SEQ ID NO: 58 - protein, 6 aa, Artificial Sequence
  Non-human like sequence
VEGFQC

SEQ ID NO: 59 - protein, 6 aa, Artificial Sequence
  Non-human like sequence
PCNPAD

SEQ ID NO: 60 - protein, 6 aa, Artificial Sequence
  Non-human like sequence
FNCYFP

SEQ ID NO: 61 - protein, 6 aa, Artificial Sequence
  Non-human like sequence
PCQGLE

SEQ ID NO: 62 - protein, 6 aa, Artificial Sequence
  Non-human like sequence
FNCYWP

SEQ ID NO: 63 - protein, 6 aa, Artificial Sequence
  Non-human like sequence
FQCYWP

SEQ ID NO: 64 - protein, 6 aa, Artificial Sequence
  Non-human like sequence
FSCYWP

SEQ ID NO: 65 - protein, 6 aa, Artificial Sequence
  Non-human like sequence
PLVSSQ

SEQ ID NO: 66 - protein, 6 aa, Artificial Sequence
  Non-human like sequence
SSQCVN

SEQ ID NO: 67 - protein, 6 aa, Artificial Sequence
  Non-human like sequence
VNLTTR

SEQ ID NO: 68 - protein, 6 aa, Artificial Sequence
  Non-human like sequence
LTTRTQ

SEQ ID NO: 69 - protein, 6 aa, Artificial Sequence
  Non-human like sequence
TRTQLP

SEQ ID NO: 70 - protein, 6 aa, Artificial Sequence
  Non-human like sequence
PAYTNS

SEQ ID NO: 71 - protein, 6 aa, Artificial Sequence
  Non-human like sequence
VFRSSV

SEQ ID NO: 72 - protein, 6 aa, Artificial Sequence
  Non-human like sequence
VLHSTQ

SEQ ID NO: 73 - protein, 6 aa, Artificial Sequence
  Non-human like sequence
LFLPFF

SEQ ID NO: 74 - protein, 6 aa, Artificial Sequence
  Non-human like sequence
SNVTWF

SEQ ID NO: 75 - protein, 6 aa, Artificial Sequence
  Non-human like sequence
VSGTNG

SEQ ID NO: 76 - protein, 6 aa, Artificial Sequence
  Non-human like sequence
SGTNGT

SEQ ID NO: 77 - protein, 6 aa, Artificial Sequence
  Non-human like sequence
TNGTKR

SEQ ID NO: 78 - protein, 6 aa, Artificial Sequence
  Non-human like sequence
NGTKRF

SEQ ID NO: 79 - protein, 6 aa, Artificial Sequence
  Non-human like sequence
RFDNPV

SEQ ID NO: 80 - protein, 6 aa, Artificial Sequence
  Non-human like sequence
VYFAST

SEQ ID NO: 81 - protein, 6 aa, Artificial Sequence
  Non-human like sequence
IRGWIF

SEQ ID NO: 82 - protein, 6 aa, Artificial Sequence
  Non-human like sequence
WIFGTT

SEQ ID NO: 83 - protein, 6 aa, Artificial Sequence
  Non-human like sequence
FGTTLD

SEQ ID NO: 84 - protein, 6 aa, Artificial Sequence
  Non-human like sequence
TTLDSK

SEQ ID NO: 85 - protein, 6 aa, Artificial Sequence
  Non-human like sequence
LDSKTQ

SEQ ID NO: 86 - protein, 6 aa, Artificial Sequence
  Non-human like sequence
DSKTQS

SEQ ID NO: 87 - protein, 6 aa, Artificial Sequence
  Non-human like sequence
SKTQSL

SEQ ID NO: 88 - protein, 6 aa, Artificial Sequence
  Non-human like sequence
KTQSLL

SEQ ID NO: 89 - protein, 6 aa, Artificial Sequence
  Non-human like sequence
VNNATN

SEQ ID NO: 90 - protein, 6 aa, Artificial Sequence
  Non-human like sequence
ATNVVI

SEQ ID NO: 91 - protein, 6 aa, Artificial Sequence
  Non-human like sequence
CEFQFC

SEQ ID NO: 92 - protein, 6 aa, Artificial Sequence
  Non-human like sequence
FCNDPF

SEQ ID NO: 93 - protein, 6 aa, Artificial Sequence
  Non-human like sequence
CNDPFL

SEQ ID NO: 94 - protein, 6 aa, Artificial Sequence
  Non-human like sequence
VSQPFL

SEQ ID NO: 95 - protein, 6 aa, Artificial Sequence
  Non-human like sequence
EGKQGN

SEQ ID NO: 96 - protein, 6 aa, Artificial Sequence
  Non-human like sequence
SKHTPI

SEQ ID NO: 97 - protein, 6 aa, Artificial Sequence
  Non-human like sequence
LVRDLP

SEQ ID NO: 98 - protein, 6 aa, Artificial Sequence
  Non-human like sequence
RDLPQG

SEQ ID NO: 99 - protein, 6 aa, Artificial Sequence
  Non-human like sequence
PQGFSA

SEQ ID NO: 100 - protein, 6 aa, Artificial Sequence
  Non-human like sequence
GFSALE

SEQ ID NO: 101 - protein, 6 aa, Artificial Sequence
  Non-human like sequence
VDLPIG

SEQ ID NO: 102 - protein, 6 aa, Artificial Sequence
  Non-human like sequence
PIGINI

SEQ ID NO: 103 - protein, 6 aa, Artificial Sequence
  Non-human like sequence
LTPGDS

SEQ ID NO: 104 - protein, 6 aa, Artificial Sequence
  Non-human like sequence
TPGDSS

SEQ ID NO: 105 - protein, 6 aa, Artificial Sequence
  Non-human like sequence
AAYYVG

SEQ ID NO: 106 - protein, 6 aa, Artificial Sequence
  Non-human like sequence
GYLQPR

SEQ ID NO: 107 - protein, 6 aa, Artificial Sequence
  Non-human like sequence
ALDPLS

SEQ ID NO: 108 - protein, 6 aa, Artificial Sequence
  Non-human like sequence
LDPLSE

SEQ ID NO: 109 - protein, 6 aa, Artificial Sequence
  Non-human like sequence
PLSETK

SEQ ID NO: 110 - protein, 6 aa, Artificial Sequence
  Non-human like sequence
SETKCT

SEQ ID NO: 111 - protein, 6 aa, Artificial Sequence
  Non-human like sequence
TVEKGI

SEQ ID NO: 112 - protein, 6 aa, Artificial Sequence
  Non-human like sequence
TSNFRV

SEQ ID NO: 113 - protein, 6 aa, Artificial Sequence
  Non-human like sequence
FRVQPT

SEQ ID NO: 114 - protein, 6 aa, Artificial Sequence
  Non-human like sequence
SIVRFP

SEQ ID NO: 115 - protein, 6 aa, Artificial Sequence
  Non-human like sequence
PNITNL

SEQ ID NO: 116 - protein, 6 aa, Artificial Sequence
  Non-human like sequence
NLCPFG

SEQ ID NO: 117 - protein, 6 aa, Artificial Sequence
  Non-human like sequence
APGQTG

SEQ ID NO: 118 - protein, 6 aa, Artificial Sequence
  Non-human like sequence
GCVIAW

SEQ ID NO: 119 - protein, 6 aa, Artificial Sequence
  Non-human like sequence
GGNYNY

SEQ ID NO: 120 - protein, 6 aa, Artificial Sequence
  Non-human like sequence
PFERDI

SEQ ID NO: 121 - protein, 6 aa, Artificial Sequence
  Non-human like sequence
ISTEIY

SEQ ID NO: 122 - protein, 6 aa, Artificial Sequence
  Non-human like sequence
STEIYQ

SEQ ID NO: 123 - protein, 6 aa, Artificial Sequence
  Non-human like sequence
PLQSYG

SEQ ID NO: 124 - protein, 6 aa, Artificial Sequence
  Non-human like sequence
APATVC

SEQ ID NO: 125 - protein, 6 aa, Artificial Sequence
  Non-human like sequence
PATVCG

SEQ ID NO: 126 - protein, 6 aa, Artificial Sequence
  Non-human like sequence
ATVCGP

SEQ ID NO: 127 - protein, 6 aa, Artificial Sequence
  Non-human like sequence
TVCGPK

SEQ ID NO: 128 - protein, 6 aa, Artificial Sequence
  Non-human like sequence
VCGPKK

SEQ ID NO: 129 - protein, 6 aa, Artificial Sequence
  Non-human like sequence
GPKKST

SEQ ID NO: 130 - protein, 6 aa, Artificial Sequence
  Non-human like sequence
PKKSTN

SEQ ID NO: 131 - protein, 6 aa, Artificial Sequence
  Non-human like sequence
KKSTNL

SEQ ID NO: 132 - protein, 6 aa, Artificial Sequence
  Non-human like sequence
KSTNLV

SEQ ID NO: 133 - protein, 6 aa, Artificial Sequence
  Non-human like sequence
VKNKCV

SEQ ID NO: 134 - protein, 6 aa, Artificial Sequence
  Non-human like sequence
FNFNGL

SEQ ID NO: 135 - protein, 6 aa, Artificial Sequence
  Non-human like sequence
FNGLTG

SEQ ID NO: 136 - protein, 6 aa, Artificial Sequence
  Non-human like sequence
NGLTGT

SEQ ID NO: 137 - protein, 6 aa, Artificial Sequence
  Non-human like sequence
TGTGVL

SEQ ID NO: 138 - protein, 6 aa, Artificial Sequence
  Non-human like sequence
GTGVLT

SEQ ID NO: 139 - protein, 6 aa, Artificial Sequence
  Non-human like sequence
GVLTES

SEQ ID NO: 140 - protein, 6 aa, Artificial Sequence
  Non-human like sequence
TESNKK

SEQ ID NO: 141 - protein, 6 aa, Artificial Sequence
  Non-human like sequence
PFQQFG

SEQ ID NO: 142 - protein, 6 aa, Artificial Sequence
  Non-human like sequence
QQFGRD

SEQ ID NO: 143 - protein, 6 aa, Artificial Sequence
  Non-human like sequence
ADTTDA

SEQ ID NO: 144 - protein, 6 aa, Artificial Sequence
  Non-human like sequence
DTTDAV

SEQ ID NO: 145 - protein, 6 aa, Artificial Sequence
  Non-human like sequence
TTDAVR

SEQ ID NO: 146 - protein, 6 aa, Artificial Sequence
  Non-human like sequence
DAVRDP

SEQ ID NO: 147 - protein, 6 aa, Artificial Sequence
  Non-human like sequence
AVRDPQ

SEQ ID NO: 148 - protein, 6 aa, Artificial Sequence
  Non-human like sequence
DITPCS

SEQ ID NO: 149 - protein, 6 aa, Artificial Sequence
  Non-human like sequence
ITPCSF

SEQ ID NO: 150 - protein, 6 aa, Artificial Sequence
  Non-human like sequence
TPCSFG

SEQ ID NO: 151 - protein, 6 aa, Artificial Sequence
  Non-human like sequence
FGGVSV

SEQ ID NO: 152 - protein, 6 aa, Artificial Sequence
  Non-human like sequence
GGVSVI

SEQ ID NO: 153 - protein, 6 aa, Artificial Sequence
  Non-human like sequence
SVITPG

SEQ ID NO: 154 - protein, 6 aa, Artificial Sequence
  Non-human like sequence
ITPGTN

SEQ ID NO: 155 - protein, 6 aa, Artificial Sequence
  Non-human like sequence
TPGTNT

SEQ ID NO: 156 - protein, 6 aa, Artificial Sequence
  Non-human like sequence
PGTNTS

SEQ ID NO: 157 - protein, 6 aa, Artificial Sequence
  Non-human like sequence
TSNQVA

SEQ ID NO: 158 - protein, 6 aa, Artificial Sequence
  Non-human like sequence
VAVLYQ

SEQ ID NO: 159 - protein, 6 aa, Artificial Sequence
  Non-human like sequence
QLTPTW

SEQ ID NO: 160 - protein, 6 aa, Artificial Sequence
  Non-human like sequence
STGSNV

SEQ ID NO: 161 - protein, 6 aa, Artificial Sequence
  Non-human like sequence
GSNVFQ

SEQ ID NO: 162 - protein, 6 aa, Artificial Sequence
  Non-human like sequence
FQTRAG

SEQ ID NO: 163 - protein, 6 aa, Artificial Sequence
  Non-human like sequence
QTRAGC

SEQ ID NO: 164 - protein, 6 aa, Artificial Sequence
  Non-human like sequence
RAGCLI

SEQ ID NO: 165 - protein, 6 aa, Artificial Sequence
  Non-human like sequence
AGCLIG

SEQ ID NO: 166 - protein, 6 aa, Artificial Sequence
  Non-human like sequence
AEHVNN

SEQ ID NO: 167 - protein, 6 aa, Artificial Sequence
  Non-human like sequence
IPIGAG

SEQ ID NO: 168 - protein, 6 aa, Artificial Sequence
  Non-human like sequence
AGICAS

SEQ ID NO: 169 - protein, 6 aa, Artificial Sequence
  Non-human like sequence
SYQTQT

SEQ ID NO: 170 - protein, 6 aa, Artificial Sequence
  Non-human like sequence
QTQTNS

SEQ ID NO: 171 - protein, 6 aa, Artificial Sequence
  Non-human like sequence
TQTNSP

SEQ ID NO: 172 - protein, 6 aa, Artificial Sequence
  Non-human like sequence
TNSPRR

SEQ ID NO: 173 - protein, 6 aa, Artificial Sequence
  Non-human like sequence
SQSIIA

SEQ ID NO: 174 - protein, 6 aa, Artificial Sequence
  Non-human like sequence
MSLGAE

SEQ ID NO: 175 - protein, 6 aa, Artificial Sequence
  Non-human like sequence
SIAIPT

SEQ ID NO: 176 - protein, 6 aa, Artificial Sequence
  Non-human like sequence
TNFTIS

SEQ ID NO: 177 - protein, 6 aa, Artificial Sequence
  Non-human like sequence
SVTTEI

SEQ ID NO: 178 - protein, 6 aa, Artificial Sequence
  Non-human like sequence
VTTEIL

SEQ ID NO: 179 - protein, 6 aa, Artificial Sequence
  Non-human like sequence
TTEILP

SEQ ID NO: 180 - protein, 6 aa, Artificial Sequence
  Non-human like sequence
TKTSVD

SEQ ID NO: 181 - protein, 6 aa, Artificial Sequence
  Non-human like sequence
KTSVDC

SEQ ID NO: 182 - protein, 6 aa, Artificial Sequence
  Non-human like sequence
TSVDCT

SEQ ID NO: 183 - protein, 6 aa, Artificial Sequence
  Non-human like sequence
YICGDS

SEQ ID NO: 184 - protein, 6 aa, Artificial Sequence
  Non-human like sequence
ICGDST

SEQ ID NO: 185 - protein, 6 aa, Artificial Sequence
  Non-human like sequence
CGDSTE

SEQ ID NO: 186 - protein, 6 aa, Artificial Sequence
  Non-human like sequence
GDSTEC

SEQ ID NO: 187 - protein, 6 aa, Artificial Sequence
  Non-human like sequence
DSTECS

SEQ ID NO: 188 - protein, 6 aa, Artificial Sequence
  Non-human like sequence
TECSNL

SEQ ID NO: 189 - protein, 6 aa, Artificial Sequence
  Non-human like sequence
LQYGSF

SEQ ID NO: 190 - protein, 6 aa, Artificial Sequence
  Non-human like sequence
GSFCTQ

SEQ ID NO: 191 - protein, 6 aa, Artificial Sequence
  Non-human like sequence
SFCTQL

SEQ ID NO: 192 - protein, 6 aa, Artificial Sequence
  Non-human like sequence
CTQLNR

SEQ ID NO: 193 - protein, 6 aa, Artificial Sequence
  Non-human like sequence
RALTGI

SEQ ID NO: 194 - protein, 6 aa, Artificial Sequence
  Non-human like sequence
ALTGIA

SEQ ID NO: 195 - protein, 6 aa, Artificial Sequence
  Non-human like sequence
VFAQVK

SEQ ID NO: 196 - protein, 6 aa, Artificial Sequence
  Non-human like sequence
QVKQIY

SEQ ID NO: 197 - protein, 6 aa, Artificial Sequence
  Non-human like sequence
QIYKTP

SEQ ID NO: 198 - protein, 6 aa, Artificial Sequence
  Non-human like sequence
YKTPPI

SEQ ID NO: 199 - protein, 6 aa, Artificial Sequence
  Non-human like sequence
PIKDFG

SEQ ID NO: 200 - protein, 6 aa, Artificial Sequence
  Non-human like sequence
FGGFNF

SEQ ID NO: 201 - protein, 6 aa, Artificial Sequence
  Non-human like sequence
GGFNFS

SEQ ID NO: 202 - protein, 6 aa, Artificial Sequence
  Non-human like sequence
PSKPSK

SEQ ID NO: 203 - protein, 6 aa, Artificial Sequence
  Non-human like sequence
SKPSKR

SEQ ID NO: 204 - protein, 6 aa, Artificial Sequence
  Non-human like sequence
KPSKRS

SEQ ID NO: 205 - protein, 6 aa, Artificial Sequence
  Non-human like sequence
PSKRSF

SEQ ID NO: 206 - protein, 6 aa, Artificial Sequence
  Non-human like sequence
NKVTLA

SEQ ID NO: 207 - protein, 6 aa, Artificial Sequence
  Non-human like sequence
KVTLAD

SEQ ID NO: 208 - protein, 6 aa, Artificial Sequence
  Non-human like sequence
DAGFIK

SEQ ID NO: 209 - protein, 6 aa, Artificial Sequence
  Non-human like sequence
AGFIKQ

SEQ ID NO: 210 - protein, 6 aa, Artificial Sequence
  Non-human like sequence
GFIKQY

SEQ ID NO: 211 - protein, 6 aa, Artificial Sequence
  Non-human like sequence
FIKQYG

SEQ ID NO: 212 - protein, 6 aa, Artificial Sequence
  Non-human like sequence
DCLGDI

SEQ ID NO: 213 - protein, 6 aa, Artificial Sequence
  Non-human like sequence
CLGDIA

SEQ ID NO: 214 - protein, 6 aa, Artificial Sequence
  Non-human like sequence
LICAQK

SEQ ID NO: 215 - protein, 6 aa, Artificial Sequence
  Non-human like sequence
AQKFNG

SEQ ID NO: 216 - protein, 6 aa, Artificial Sequence
  Non-human like sequence
QKFNGL

SEQ ID NO: 217 - protein, 6 aa, Artificial Sequence
  Non-human like sequence
NGLTVL

SEQ ID NO: 218 - protein, 6 aa, Artificial Sequence
  Non-human like sequence
LLTDEM

SEQ ID NO: 219 - protein, 6 aa, Artificial Sequence
  Non-human like sequence
SGWTFG

SEQ ID NO: 220 - protein, 6 aa, Artificial Sequence
  Non-human like sequence
GWTFGA

SEQ ID NO: 221 - protein, 6 aa, Artificial Sequence
  Non-human like sequence
WTFGAG

SEQ ID NO: 222 - protein, 6 aa, Artificial Sequence
  Non-human like sequence
TFGAGA

SEQ ID NO: 223 - protein, 6 aa, Artificial Sequence
  Non-human like sequence
IPFAMQ

SEQ ID NO: 224 - protein, 6 aa, Artificial Sequence
  Non-human like sequence
MAYRFN

SEQ ID NO: 225 - protein, 6 aa, Artificial Sequence
  Non-human like sequence
RFNGIG

SEQ ID NO: 226 - protein, 6 aa, Artificial Sequence
  Non-human like sequence
NGIGVT

SEQ ID NO: 227 - protein, 6 aa, Artificial Sequence
  Non-human like sequence
GIGVTQ

SEQ ID NO: 228 - protein, 6 aa, Artificial Sequence
  Non-human like sequence
IGVTQN

SEQ ID NO: 229 - protein, 6 aa, Artificial Sequence
  Non-human like sequence
QKLIAN

SEQ ID NO: 230 - protein, 6 aa, Artificial Sequence
  Non-human like sequence
KLIANQ

SEQ ID NO: 231 - protein, 6 aa, Artificial Sequence
  Non-human like sequence
LIANQF

SEQ ID NO: 232 - protein, 6 aa, Artificial Sequence
  Non-human like sequence
QFNSAI

SEQ ID NO: 233 - protein, 6 aa, Artificial Sequence
  Non-human like sequence
NSAIGK

SEQ ID NO: 234 - protein, 6 aa, Artificial Sequence
  Non-human like sequence
AIGKIQ

SEQ ID NO: 235 - protein, 6 aa, Artificial Sequence
  Non-human like sequence
ALGKLQ

SEQ ID NO: 236 - protein, 6 aa, Artificial Sequence
  Non-human like sequence
NQNAQA

SEQ ID NO: 237 - protein, 6 aa, Artificial Sequence
  Non-human like sequence
ALNTLV

SEQ ID NO: 238 - protein, 6 aa, Artificial Sequence
  Non-human like sequence
NFGAIS

SEQ ID NO: 239 - protein, 6 aa, Artificial Sequence
  Non-human like sequence
FGAISS

SEQ ID NO: 240 - protein, 6 aa, Artificial Sequence
  Non-human like sequence
AISSVL

SEQ ID NO: 241 - protein, 6 aa, Artificial Sequence
  Non-human like sequence
ILSRLD

SEQ ID NO: 242 - protein, 6 aa, Artificial Sequence
  Non-human like sequence
DRLITG

SEQ ID NO: 243 - protein, 6 aa, Artificial Sequence
  Non-human like sequence
ITGRLQ

SEQ ID NO: 244 - protein, 6 aa, Artificial Sequence
  Non-human like sequence
IRASAN

SEQ ID NO: 245 - protein, 6 aa, Artificial Sequence
  Non-human like sequence
NLAATK

SEQ ID NO: 246 - protein, 6 aa, Artificial Sequence
  Non-human like sequence
CVLGQS

SEQ ID NO: 247 - protein, 6 aa, Artificial Sequence
  Non-human like sequence
VLGQSK

SEQ ID NO: 248 - protein, 6 aa, Artificial Sequence
  Non-human like sequence
LGQSKR

SEQ ID NO: 249 - protein, 6 aa, Artificial Sequence
  Non-human like sequence
GQSKRV

SEQ ID NO: 250 - protein, 6 aa, Artificial Sequence
  Non-human like sequence
QSKRVD

SEQ ID NO: 251 - protein, 6 aa, Artificial Sequence
  Non-human like sequence
RVDFCG

SEQ ID NO: 252 - protein, 6 aa, Artificial Sequence
  Non-human like sequence
FCGKGY

SEQ ID NO: 253 - protein, 6 aa, Artificial Sequence
  Non-human like sequence
FPQSAP

SEQ ID NO: 254 - protein, 6 aa, Artificial Sequence
  Non-human like sequence
QSAPHG

SEQ ID NO: 255 - protein, 6 aa, Artificial Sequence
  Non-human like sequence
SAPHGV

SEQ ID NO: 256 - protein, 6 aa, Artificial Sequence
  Non-human like sequence
APHGVV

SEQ ID NO: 257 - protein, 6 aa, Artificial Sequence
  Non-human like sequence
HGVVFL

SEQ ID NO: 258 - protein, 6 aa, Artificial Sequence
  Non-human like sequence
VPAQEK

SEQ ID NO: 259 - protein, 6 aa, Artificial Sequence
  Non-human like sequence
KNFTTA

SEQ ID NO: 260 - protein, 6 aa, Artificial Sequence
  Non-human like sequence
FTTAPA

SEQ ID NO: 261 - protein, 6 aa, Artificial Sequence
  Non-human like sequence
CHDGKA

SEQ ID NO: 262 - protein, 6 aa, Artificial Sequence
  Non-human like sequence
HDGKAH

SEQ ID NO: 263 - protein, 6 aa, Artificial Sequence
  Non-human like sequence
KAHFPR

SEQ ID NO: 264 - protein, 6 aa, Artificial Sequence
  Non-human like sequence
GVFVSN

SEQ ID NO: 265 - protein, 6 aa, Artificial Sequence
  Non-human like sequence
SNGTHW

SEQ ID NO: 266 - protein, 6 aa, Artificial Sequence
  Non-human like sequence
NGTHWF

SEQ ID NO: 267 - protein, 6 aa, Artificial Sequence
  Non-human like sequence
GTHWFV

SEQ ID NO: 268 - protein, 6 aa, Artificial Sequence
  Non-human like sequence
QRNFYE

SEQ ID NO: 269 - protein, 6 aa, Artificial Sequence
  Non-human like sequence
FYEPQI

SEQ ID NO: 270 - protein, 6 aa, Artificial Sequence
  Non-human like sequence
YEPQII

SEQ ID NO: 271 - protein, 6 aa, Artificial Sequence
  Non-human like sequence
TFVSGN

SEQ ID NO: 272 - protein, 6 aa, Artificial Sequence
  Non-human like sequence
SGNCDV

SEQ ID NO: 273 - protein, 6 aa, Artificial Sequence
  Non-human like sequence
GIVNNT

SEQ ID NO: 274 - protein, 6 aa, Artificial Sequence
  Non-human like sequence
NNTVYD

SEQ ID NO: 275 - protein, 6 aa, Artificial Sequence
  Non-human like sequence
QPELDS

SEQ ID NO: 276 - protein, 6 aa, Artificial Sequence
  Non-human like sequence
PELDSF

SEQ ID NO: 277 - protein, 6 aa, Artificial Sequence
  Non-human like sequence
LDSFKE

SEQ ID NO: 278 - protein, 6 aa, Artificial Sequence
  Non-human like sequence
TSPDVD

SEQ ID NO: 279 - protein, 6 aa, Artificial Sequence
  Non-human like sequence
RLNEVA

SEQ ID NO: 280 - protein, 6 aa, Artificial Sequence
  Non-human like sequence
NEVAKN

SEQ ID NO: 281 - protein, 6 aa, Artificial Sequence
  Non-human like sequence
YIKWPW

SEQ ID NO: 282 - protein, 6 aa, Artificial Sequence
  Non-human like sequence
LIAIVM

SEQ ID NO: 283 - protein, 6 aa, Artificial Sequence
  Non-human like sequence
SCLKGC

SEQ ID NO: 284 - protein, 6 aa, Artificial Sequence
  Non-human like sequence
CLKGCC

SEQ ID NO: 285 - protein, 6 aa, Artificial Sequence
  Non-human like sequence
CCKFDE

SEQ ID NO: 286 - protein, 6 aa, Artificial Sequence
  Non-human like sequence
EPVLKG

SEQ ID NO: 287 - protein, 6 aa, Artificial Sequence
  Non-human like sequence
KGVKLH

SEQ ID NO: 288 - protein, 6 aa, Artificial Sequence
  Non-human like sequence
GVKLHY

SEQ ID NO: 289 - protein, 6 aa, Artificial Sequence
  Non-human like sequence
ATVTGP

SEQ ID NO: 290 - protein, 6 aa, Artificial Sequence
  Non-human like sequence
VKNKSV

SEQ ID NO: 291 - protein, 419 aa, SARS-CoV-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 292 - protein, 9 aa, Artificial Sequence
  Non-human like sequence
FYPRGQGVF

SEQ ID NO: 293 - protein, 9 aa, Artificial Sequence
  Non-human like sequence
ATNTASWFR

SEQ ID NO: 294 - protein, 9 aa, Artificial Sequence
  Non-human like sequence
FQFPRGQGI

SEQ ID NO: 295 - protein, 6 aa, Artificial Sequence
  Non-human like sequence
MSDNGP

SEQ ID NO: 296 - protein, 6 aa, Artificial Sequence
  Non-human like sequence
NAPRIT

SEQ ID NO: 297 - protein, 6 aa, Artificial Sequence
  Non-human like sequence
RITFGG

SEQ ID NO: 298 - protein, 6 aa, Artificial Sequence
  Non-human like sequence
ITFGGP

SEQ ID NO: 299 - protein, 6 aa, Artificial Sequence
  Non-human like sequence
GGPSDS

SEQ ID NO: 300 - protein, 6 aa, Artificial Sequence
  Non-human like sequence
GPSDST

SEQ ID NO: 301 - protein, 6 aa, Artificial Sequence
  Non-human like sequence
DSTGSN

SEQ ID NO: 302 - protein, 6 aa, Artificial Sequence
  Non-human like sequence
STGSNQ

SEQ ID NO: 303 - protein, 6 aa, Artificial Sequence
  Non-human like sequence
TGSNQN

SEQ ID NO: 304 - protein, 6 aa, Artificial Sequence
  Non-human like sequence
GSNQNG

SEQ ID NO: 305 - protein, 6 aa, Artificial Sequence
  Non-human like sequence
SNQNGE

SEQ ID NO: 306 - protein, 6 aa, Artificial Sequence
  Non-human like sequence
GARSKQ

SEQ ID NO: 307 - protein, 6 aa, Artificial Sequence
  Non-human like sequence
SKQRRP

SEQ ID NO: 308 - protein, 6 aa, Artificial Sequence
  Non-human like sequence
LPNNTA

SEQ ID NO: 309 - protein, 6 aa, Artificial Sequence
  Non-human like sequence
NTASWF

SEQ ID NO: 310 - protein, 6 aa, Artificial Sequence
  Non-human like sequence
TASWFT

SEQ ID NO: 311 - protein, 6 aa, Artificial Sequence
  Non-human like sequence
SWFTAL

SEQ ID NO: 312 - protein, 6 aa, Artificial Sequence
  Non-human like sequence
FTALTQ

SEQ ID NO: 313 - protein, 6 aa, Artificial Sequence
  Non-human like sequence
LTQHGK

SEQ ID NO: 314 - protein, 6 aa, Artificial Sequence
  Non-human like sequence
TQHGKE

SEQ ID NO: 315 - protein, 6 aa, Artificial Sequence
  Non-human like sequence
HGKEDL

SEQ ID NO: 316 - protein, 6 aa, Artificial Sequence
  Non-human like sequence
DLKFPR

SEQ ID NO: 317 - protein, 6 aa, Artificial Sequence
  Non-human like sequence
LKFPRG

SEQ ID NO: 318 - protein, 6 aa, Artificial Sequence
  Non-human like sequence
KFPRGQ

SEQ ID NO: 319 - protein, 6 aa, Artificial Sequence
  Non-human like sequence
FPRGQG

SEQ ID NO: 320 - protein, 6 aa, Artificial Sequence
  Non-human like sequence
PRGQGV

SEQ ID NO: 321 - protein, 6 aa, Artificial Sequence
  Non-human like sequence
RGQGVP

SEQ ID NO: 322 - protein, 6 aa, Artificial Sequence
  Non-human like sequence
PINTNS

SEQ ID NO: 323 - protein, 6 aa, Artificial Sequence
  Non-human like sequence
INTNSS

SEQ ID NO: 324 - protein, 6 aa, Artificial Sequence
  Non-human like sequence
TNSSPD

SEQ ID NO: 325 - protein, 6 aa, Artificial Sequence
  Non-human like sequence
QIGYYR

SEQ ID NO: 326 - protein, 6 aa, Artificial Sequence
  Non-human like sequence
YRRATR

SEQ ID NO: 327 - protein, 6 aa, Artificial Sequence
  Non-human like sequence
TRRIRG

SEQ ID NO: 328 - protein, 6 aa, Artificial Sequence
  Non-human like sequence
RIRGGD

SEQ ID NO: 329 - protein, 6 aa, Artificial Sequence
  Non-human like sequence
IRGGDG

SEQ ID NO: 330 - protein, 6 aa, Artificial Sequence
  Non-human like sequence
RGGDGK

SEQ ID NO: 331 - protein, 6 aa, Artificial Sequence
  Non-human like sequence
WYFYYL

SEQ ID NO: 332 - protein, 6 aa, Artificial Sequence
  Non-human like sequence
YFYYLG

SEQ ID NO: 333 - protein, 6 aa, Artificial Sequence
  Non-human like sequence
LGTGPE

SEQ ID NO: 334 - protein, 6 aa, Artificial Sequence
  Non-human like sequence
GANKDG

SEQ ID NO: 335 - protein, 6 aa, Artificial Sequence
  Non-human like sequence
ANKDGI

SEQ ID NO: 336 - protein, 6 aa, Artificial Sequence
  Non-human like sequence
KDGIIW

SEQ ID NO: 337 - protein, 6 aa, Artificial Sequence
  Non-human like sequence
TEGALN

SEQ ID NO: 338 - protein, 6 aa, Artificial Sequence
  Non-human like sequence
EGALNT

SEQ ID NO: 339 - protein, 6 aa, Artificial Sequence
  Non-human like sequence
NTPKDH

SEQ ID NO: 340 - protein, 6 aa, Artificial Sequence
  Non-human like sequence
PKDHIG

SEQ ID NO: 341 - protein, 6 aa, Artificial Sequence
  Non-human like sequence
HIGTRN

SEQ ID NO: 342 - protein, 6 aa, Artificial Sequence
  Non-human like sequence
GTRNPA

SEQ ID NO: 343 - protein, 6 aa, Artificial Sequence
  Non-human like sequence
AAIVLQ

SEQ ID NO: 344 - protein, 6 aa, Artificial Sequence
  Non-human like sequence
AIVLQL

SEQ ID NO: 345 - protein, 6 aa, Artificial Sequence
  Non-human like sequence
PQGTTL

SEQ ID NO: 346 - protein, 6 aa, Artificial Sequence
  Non-human like sequence
GTTLPK

SEQ ID NO: 347 - protein, 6 aa, Artificial Sequence
  Non-human like sequence
TTLPKG

SEQ ID NO: 348 - protein, 6 aa, Artificial Sequence
  Non-human like sequence
FYAEGS

SEQ ID NO: 349 - protein, 6 aa, Artificial Sequence
  Non-human like sequence
SQASSR

SEQ ID NO: 350 - protein, 6 aa, Artificial Sequence
  Non-human like sequence
RSRNSS

SEQ ID NO: 351 - protein, 6 aa, Artificial Sequence
  Non-human like sequence
SRNSTP

SEQ ID NO: 352 - protein, 6 aa, Artificial Sequence
  Non-human like sequence
RNSTPG

SEQ ID NO: 353 - protein, 6 aa, Artificial Sequence
  Non-human like sequence
NSTPGS

SEQ ID NO: 354 - protein, 6 aa, Artificial Sequence
  Non-human like sequence
GSSRGT

SEQ ID NO: 355 - protein, 6 aa, Artificial Sequence
  Non-human like sequence
SRGTSP

SEQ ID NO: 356 - protein, 6 aa, Artificial Sequence
  Non-human like sequence
RGTSPA

SEQ ID NO: 357 - protein, 6 aa, Artificial Sequence
  Non-human like sequence
GTSPAR

SEQ ID NO: 358 - protein, 6 aa, Artificial Sequence
  Non-human like sequence
RMAGNG

SEQ ID NO: 359 - protein, 6 aa, Artificial Sequence
  Non-human like sequence
MAGNGG

SEQ ID NO: 360 - protein, 6 aa, Artificial Sequence
  Non-human like sequence
AGNGGD

SEQ ID NO: 361 - protein, 6 aa, Artificial Sequence
  Non-human like sequence
GNGGDA

SEQ ID NO: 362 - protein, 6 aa, Artificial Sequence
  Non-human like sequence
GDAALA

SEQ ID NO: 363 - protein, 6 aa, Artificial Sequence
  Non-human like sequence
DRLNQL

SEQ ID NO: 364 - protein, 6 aa, Artificial Sequence
  Non-human like sequence
NQLESK

SEQ ID NO: 365 - protein, 6 aa, Artificial Sequence
  Non-human like sequence
ESKMSG

SEQ ID NO: 366 - protein, 6 aa, Artificial Sequence
  Non-human like sequence
MSGKGQ

SEQ ID NO: 367 - protein, 6 aa, Artificial Sequence
  Non-human like sequence
SGKGQQ

SEQ ID NO: 368 - protein, 6 aa, Artificial Sequence
  Non-human like sequence
QGQTVT

SEQ ID NO: 369 - protein, 6 aa, Artificial Sequence
  Non-human like sequence
GQTVTK

SEQ ID NO: 370 - protein, 6 aa, Artificial Sequence
  Non-human like sequence
TVTKKS

SEQ ID NO: 371 - protein, 6 aa, Artificial Sequence
  Non-human like sequence
KSAAEA

SEQ ID NO: 372 - protein, 6 aa, Artificial Sequence
  Non-human like sequence
AEASKK

SEQ ID NO: 373 - protein, 6 aa, Artificial Sequence
  Non-human like sequence
EASKKP

SEQ ID NO: 374 - protein, 6 aa, Artificial Sequence
  Non-human like sequence
PRQKRT

SEQ ID NO: 375 - protein, 6 aa, Artificial Sequence
  Non-human like sequence
KRTATK

SEQ ID NO: 376 - protein, 6 aa, Artificial Sequence
  Non-human like sequence
RTATKA

SEQ ID NO: 377 - protein, 6 aa, Artificial Sequence
  Non-human like sequence
VTQAFG

SEQ ID NO: 378 - protein, 6 aa, Artificial Sequence
  Non-human like sequence
TQAFGR

SEQ ID NO: 379 - protein, 6 aa, Artificial Sequence
  Non-human like sequence
AFGRRG

SEQ ID NO: 380 - protein, 6 aa, Artificial Sequence
  Non-human like sequence
GRRGPE

SEQ ID NO: 381 - protein, 6 aa, Artificial Sequence
  Non-human like sequence
TQGNFG

SEQ ID NO: 382 - protein, 6 aa, Artificial Sequence
  Non-human like sequence
GNFGDQ

SEQ ID NO: 383 - protein, 6 aa, Artificial Sequence
  Non-human like sequence
DQELIR

SEQ ID NO: 384 - protein, 6 aa, Artificial Sequence
  Non-human like sequence
LIRQGT

SEQ ID NO: 385 - protein, 6 aa, Artificial Sequence
  Non-human like sequence
RQGTDY

SEQ ID NO: 386 - protein, 6 aa, Artificial Sequence
  Non-human like sequence
QGTDYK

SEQ ID NO: 387 - protein, 6 aa, Artificial Sequence
  Non-human like sequence
AQFAPS

SEQ ID NO: 388 - protein, 6 aa, Artificial Sequence
  Non-human like sequence
PSASAF

SEQ ID NO: 389 - protein, 6 aa, Artificial Sequence
  Non-human like sequence
EVTPSG

SEQ ID NO: 390 - protein, 6 aa, Artificial Sequence
  Non-human like sequence
VTPSGT

SEQ ID NO: 391 - protein, 6 aa, Artificial Sequence
  Non-human like sequence
YTGAIK

SEQ ID NO: 392 - protein, 6 aa, Artificial Sequence
  Non-human like sequence
AIKLDD

SEQ ID NO: 393 - protein, 6 aa, Artificial Sequence
  Non-human like sequence
HIDAYK

SEQ ID NO: 394 - protein, 6 aa, Artificial Sequence
  Non-human like sequence
AYKTFP

SEQ ID NO: 395 - protein, 6 aa, Artificial Sequence
  Non-human like sequence
YKTFPP

SEQ ID NO: 396 - protein, 6 aa, Artificial Sequence
  Non-human like sequence
KKKADE

SEQ ID NO: 397 - protein, 6 aa, Artificial Sequence
  Non-human like sequence
KKADET

SEQ ID NO: 398 - protein, 6 aa, Artificial Sequence
  Non-human like sequence
QALPQR

SEQ ID NO: 399 - protein, 6 aa, Artificial Sequence
  Non-human like sequence
SADSTQ

SEQ ID NO: 400 - protein, 6 aa, Artificial Sequence
  Non-human like sequence
ADSTQA

SEQ ID NO: 401 - protein, 9 aa, Artificial Sequence
  Non-human like sequence
ATNTASWFD

SEQ ID NO: 402 - protein, 9 aa, Artificial Sequence
  Non-human like sequence
ATNTASWFF

SEQ ID NO: 403 - protein, 9 aa, Artificial Sequence
  Non-human like sequence
ATNTASWFK

SEQ ID NO: 404 - protein, 9 aa, Artificial Sequence
  Non-human like sequence
ATNTASWFN

SEQ ID NO: 405 - protein, 9 aa, Artificial Sequence
  Non-human like sequence
ATNTASWFR

SEQ ID NO: 406 - protein, 9 aa, Artificial Sequence
  Non-human like sequence
ATNTASWFS

SEQ ID NO: 407 - protein, 9 aa, Artificial Sequence
  Non-human like sequence
GGNTASWFR

SEQ ID NO: 408 - protein, 9 aa, Artificial Sequence
  Non-human like sequence
GGNTASWFS

SEQ ID NO: 409 - protein, 9 aa, Artificial Sequence
  Non-human like sequence
GTNTASWFR

SEQ ID NO: 410 - protein, 9 aa, Artificial Sequence
  Non-human like sequence
GTNTASWFS

SEQ ID NO: 411 - protein, 9 aa, Artificial Sequence
  Non-human like sequence
GNTASWFTG

SEQ ID NO: 412 - protein, 9 aa, Artificial Sequence
  Non-human like sequence
GTTASWFTG

SEQ ID NO: 413 - protein, 9 aa, Artificial Sequence
  Non-human like sequence
TNTASWFTG

SEQ ID NO: 414 - protein, 9 aa, Artificial Sequence
  Non-human like sequence
KGFTALTQS

SEQ ID NO: 415 - protein, 9 aa, Artificial Sequence
  Non-human like sequence
RGFTALTQS

SEQ ID NO: 416 - protein, 9 aa, Artificial Sequence
  Non-human like sequence
ASTQHGKER

SEQ ID NO: 417 - protein, 9 aa, Artificial Sequence
  Non-human like sequence
EGTQHGKEN

SEQ ID NO: 418 - protein, 9 aa, Artificial Sequence
  Non-human like sequence
ESTQHGKEN

SEQ ID NO: 419 - protein, 9 aa, Artificial Sequence
  Non-human like sequence
FSTQHGKEN

SEQ ID NO: 420 - protein, 9 aa, Artificial Sequence
  Non-human like sequence
GGTQHGKEN

SEQ ID NO: 421 - protein, 9 aa, Artificial Sequence
  Non-human like sequence
GHTQHGKEN

SEQ ID NO: 422 - protein, 9 aa, Artificial Sequence
  Non-human like sequence
IGTQHGKEN

SEQ ID NO: 423 - protein, 9 aa, Artificial Sequence
  Non-human like sequence
ISTQHGKEN

SEQ ID NO: 424 - protein, 9 aa, Artificial Sequence
  Non-human like sequence
ITTQHGKEN

SEQ ID NO: 425 - protein, 9 aa, Artificial Sequence
  Non-human like sequence
LTTQHGKEN

SEQ ID NO: 426 - protein, 9 aa, Artificial Sequence
  Non-human like sequence
MGTQHGKEN

SEQ ID NO: 427 - protein, 9 aa, Artificial Sequence
  Non-human like sequence
NGTQHGKEN

SEQ ID NO: 428 - protein, 9 aa, Artificial Sequence
  Non-human like sequence
QSTQHGKEN

SEQ ID NO: 429 - protein, 9 aa, Artificial Sequence
  Non-human like sequence
QSTQHGKER

SEQ ID NO: 430 - protein, 9 aa, Artificial Sequence
  Non-human like sequence
RHTQHGKEN

SEQ ID NO: 431 - protein, 9 aa, Artificial Sequence
  Non-human like sequence
RSTQHGKEN

SEQ ID NO: 432 - protein, 9 aa, Artificial Sequence
  Non-human like sequence
RTTQHGKEN

SEQ ID NO: 433 - protein, 9 aa, Artificial Sequence
  Non-human like sequence
STTQHGKEN

SEQ ID NO: 434 - protein, 9 aa, Artificial Sequence
  Non-human like sequence
STTQHGKER

SEQ ID NO: 435 - protein, 9 aa, Artificial Sequence
  Non-human like sequence
TTTQHGKEN

SEQ ID NO: 436 - protein, 9 aa, Artificial Sequence
  Non-human like sequence
YGTQHGKEN

SEQ ID NO: 437 - protein, 9 aa, Artificial Sequence
  Non-human like sequence
DALKFPRGQ

SEQ ID NO: 438 - protein, 9 aa, Artificial Sequence
  Non-human like sequence
EYLKFPRGH

SEQ ID NO: 439 - protein, 9 aa, Artificial Sequence
  Non-human like sequence
PALKFPRGQ

SEQ ID NO: 440 - protein, 9 aa, Artificial Sequence
  Non-human like sequence
QYLKFPRGH

SEQ ID NO: 441 - protein, 9 aa, Artificial Sequence
  Non-human like sequence
SNLKFPRGH

SEQ ID NO: 442 - protein, 9 aa, Artificial Sequence
  Non-human like sequence
SNLKFPRGQ

SEQ ID NO: 443 - protein, 9 aa, Artificial Sequence
  Non-human like sequence
ADKFPRGQL

SEQ ID NO: 444 - protein, 9 aa, Artificial Sequence
  Non-human like sequence
ADKFPRGQS

SEQ ID NO: 445 - protein, 9 aa, Artificial Sequence
  Non-human like sequence
AKKFPRGQH

SEQ ID NO: 446 - protein, 9 aa, Artificial Sequence
  Non-human like sequence
AKKFPRGQL

SEQ ID NO: 447 - protein, 9 aa, Artificial Sequence
  Non-human like sequence
AKKFPRGQN

SEQ ID NO: 448 - protein, 9 aa, Artificial Sequence
  Non-human like sequence
AKKFPRGQP

SEQ ID NO: 449 - protein, 9 aa, Artificial Sequence
  Non-human like sequence
ALKFPRGQL

SEQ ID NO: 450 - protein, 9 aa, Artificial Sequence
  Non-human like sequence
ALKFPRGQP

SEQ ID NO: 451 - protein, 9 aa, Artificial Sequence
  Non-human like sequence
ALKFPRGQS

SEQ ID NO: 452 - protein, 9 aa, Artificial Sequence
  Non-human like sequence
CSKFPRGQH

SEQ ID NO: 453 - protein, 9 aa, Artificial Sequence
  Non-human like sequence
CSKFPRGQL

SEQ ID NO: 454 - protein, 9 aa, Artificial Sequence
  Non-human like sequence
CSKFPRGQP

SEQ ID NO: 455 - protein, 9 aa, Artificial Sequence
  Non-human like sequence
CSKFPRGQS

SEQ ID NO: 456 - protein, 9 aa, Artificial Sequence
  Non-human like sequence
DCKFPRGQH

SEQ ID NO: 457 - protein, 9 aa, Artificial Sequence
  Non-human like sequence
DCKFPRGQP

SEQ ID NO: 458 - protein, 9 aa, Artificial Sequence
  Non-human like sequence
FKKFPRGQH

SEQ ID NO: 459 - protein, 9 aa, Artificial Sequence
  Non-human like sequence
FKKFPRGQL

SEQ ID NO: 460 - protein, 9 aa, Artificial Sequence
  Non-human like sequence
FKKFPRGQN

SEQ ID NO: 461 - protein, 9 aa, Artificial Sequence
  Non-human like sequence
FKKFPRGQS

SEQ ID NO: 462 - protein, 9 aa, Artificial Sequence
  Non-human like sequence
GAKFPRGQC

SEQ ID NO: 463 - protein, 9 aa, Artificial Sequence
  Non-human like sequence
GAKFPRGQH

SEQ ID NO: 464 - protein, 9 aa, Artificial Sequence
  Non-human like sequence
GAKFPRGQL

SEQ ID NO: 465 - protein, 9 aa, Artificial Sequence
  Non-human like sequence
GAKFPRGQS

SEQ ID NO: 466 - protein, 9 aa, Artificial Sequence
  Non-human like sequence
GCKFPRGQH

SEQ ID NO: 467 - protein, 9 aa, Artificial Sequence
  Non-human like sequence
GCKFPRGQL

SEQ ID NO: 468 - protein, 9 aa, Artificial Sequence
  Non-human like sequence
GCKFPRGQN

SEQ ID NO: 469 - protein, 9 aa, Artificial Sequence
  Non-human like sequence
GCKFPRGQP

SEQ ID NO: 470 - protein, 9 aa, Artificial Sequence
  Non-human like sequence
GCKFPRGQS

SEQ ID NO: 471 - protein, 9 aa, Artificial Sequence
  Non-human like sequence
GSKFPRGQC

SEQ ID NO: 472 - protein, 9 aa, Artificial Sequence
  Non-human like sequence
GSKFPRGQH

SEQ ID NO: 473 - protein, 9 aa, Artificial Sequence
  Non-human like sequence
GSKFPRGQP

SEQ ID NO: 474 - protein, 9 aa, Artificial Sequence
  Non-human like sequence
GSKFPRGQQ

SEQ ID NO: 475 - protein, 9 aa, Artificial Sequence
  Non-human like sequence
GSKFPRGQS

SEQ ID NO: 476 - protein, 9 aa, Artificial Sequence
  Non-human like sequence
HAKFPRGQC

SEQ ID NO: 477 - protein, 9 aa, Artificial Sequence
  Non-human like sequence
HAKFPRGQH

SEQ ID NO: 478 - protein, 9 aa, Artificial Sequence
  Non-human like sequence
HAKFPRGQL

SEQ ID NO: 479 - protein, 9 aa, Artificial Sequence
  Non-human like sequence
HAKFPRGQS

SEQ ID NO: 480 - protein, 9 aa, Artificial Sequence
  Non-human like sequence
HDKFPRGQH

SEQ ID NO: 481 - protein, 9 aa, Artificial Sequence
  Non-human like sequence
HDKFPRGQL

SEQ ID NO: 482 - protein, 9 aa, Artificial Sequence
  Non-human like sequence
HDKFPRGQP

SEQ ID NO: 483 - protein, 9 aa, Artificial Sequence
  Non-human like sequence
HDKFPRGQS

SEQ ID NO: 484 - protein, 9 aa, Artificial Sequence
  Non-human like sequence
HQKFPRGQG

SEQ ID NO: 485 - protein, 9 aa, Artificial Sequence
  Non-human like sequence
HQKFPRGQH

SEQ ID NO: 486 - protein, 9 aa, Artificial Sequence
  Non-human like sequence
HQKFPRGQP

SEQ ID NO: 487 - protein, 9 aa, Artificial Sequence
  Non-human like sequence
HQKFPRGQS

SEQ ID NO: 488 - protein, 9 aa, Artificial Sequence
  Non-human like sequence
HSKFPRGQH

SEQ ID NO: 489 - protein, 9 aa, Artificial Sequence
  Non-human like sequence
HSKFPRGQL

SEQ ID NO: 490 - protein, 9 aa, Artificial Sequence
  Non-human like sequence
HSKFPRGQP

SEQ ID NO: 491 - protein, 9 aa, Artificial Sequence
  Non-human like sequence
HSKFPRGQS

SEQ ID NO: 492 - protein, 9 aa, Artificial Sequence
  Non-human like sequence
IAKFPRGQC

SEQ ID NO: 493 - protein, 9 aa, Artificial Sequence
  Non-human like sequence
IAKFPRGQH

SEQ ID NO: 494 - protein, 9 aa, Artificial Sequence
  Non-human like sequence
IAKFPRGQL

SEQ ID NO: 495 - protein, 9 aa, Artificial Sequence
  Non-human like sequence
IAKFPRGQP

SEQ ID NO: 496 - protein, 9 aa, Artificial Sequence
  Non-human like sequence
IAKFPRGQS

SEQ ID NO: 497 - protein, 9 aa, Artificial Sequence
  Non-human like sequence
KAKFPRGQH

SEQ ID NO: 498 - protein, 9 aa, Artificial Sequence
  Non-human like sequence
KAKFPRGQL

SEQ ID NO: 499 - protein, 9 aa, Artificial Sequence
  Non-human like sequence
KAKFPRGQP

SEQ ID NO: 500 - protein, 9 aa, Artificial Sequence
  Non-human like sequence
KAKFPRGQS

SEQ ID NO: 501 - protein, 9 aa, Artificial Sequence
  Non-human like sequence
KKKFPRGQH

SEQ ID NO: 502 - protein, 9 aa, Artificial Sequence
  Non-human like sequence
KKKFPRGQL

SEQ ID NO: 503 - protein, 9 aa, Artificial Sequence
  Non-human like sequence
KKKFPRGQN

SEQ ID NO: 504 - protein, 9 aa, Artificial Sequence
  Non-human like sequence
KKKFPRGQP

SEQ ID NO: 505 - protein, 9 aa, Artificial Sequence
  Non-human like sequence
KKKFPRGQS

SEQ ID NO: 506 - protein, 9 aa, Artificial Sequence
  Non-human like sequence
KQKFPRGQH

SEQ ID NO: 507 - protein, 9 aa, Artificial Sequence
  Non-human like sequence
KQKFPRGQP

SEQ ID NO: 508 - protein, 9 aa, Artificial Sequence
  Non-human like sequence
KQKFPRGQS

SEQ ID NO: 509 - protein, 9 aa, Artificial Sequence
  Non-human like sequence
KSKFPRGQH

SEQ ID NO: 510 - protein, 9 aa, Artificial Sequence
  Non-human like sequence
KSKFPRGQL

SEQ ID NO: 511 - protein, 9 aa, Artificial Sequence
  Non-human like sequence
KSKFPRGQP

SEQ ID NO: 512 - protein, 9 aa, Artificial Sequence
  Non-human like sequence
KSKFPRGQS

SEQ ID NO: 513 - protein, 9 aa, Artificial Sequence
  Non-human like sequence
LAKFPRGQC

SEQ ID NO: 514 - protein, 9 aa, Artificial Sequence
  Non-human like sequence
LAKFPRGQH

SEQ ID NO: 515 - protein, 9 aa, Artificial Sequence
  Non-human like sequence
LAKFPRGQL

SEQ ID NO: 516 - protein, 9 aa, Artificial Sequence
  Non-human like sequence
LAKFPRGQP

SEQ ID NO: 517 - protein, 9 aa, Artificial Sequence
  Non-human like sequence
LAKFPRGQS

SEQ ID NO: 518 - protein, 9 aa, Artificial Sequence
  Non-human like sequence
LSKFPRGQC

SEQ ID NO: 519 - protein, 9 aa, Artificial Sequence
  Non-human like sequence
LSKFPRGQH

SEQ ID NO: 520 - protein, 9 aa, Artificial Sequence
  Non-human like sequence
LSKFPRGQL

SEQ ID NO: 521 - protein, 9 aa, Artificial Sequence
  Non-human like sequence
LSKFPRGQP

SEQ ID NO: 522 - protein, 9 aa, Artificial Sequence
  Non-human like sequence
LSKFPRGQS

SEQ ID NO: 523 - protein, 9 aa, Artificial Sequence
  Non-human like sequence
NAKFPRGQH

SEQ ID NO: 524 - protein, 9 aa, Artificial Sequence
  Non-human like sequence
NAKFPRGQL

SEQ ID NO: 525 - protein, 9 aa, Artificial Sequence
  Non-human like sequence
NAKFPRGQN

SEQ ID NO: 526 - protein, 9 aa, Artificial Sequence
  Non-human like sequence
NAKFPRGQP

SEQ ID NO: 527 - protein, 9 aa, Artificial Sequence
  Non-human like sequence
NAKFPRGQS

SEQ ID NO: 528 - protein, 9 aa, Artificial Sequence
  Non-human like sequence
NKKFPRGQG

SEQ ID NO: 529 - protein, 9 aa, Artificial Sequence
  Non-human like sequence
NKKFPRGQH

SEQ ID NO: 530 - protein, 9 aa, Artificial Sequence
  Non-human like sequence
NKKFPRGQL

SEQ ID NO: 531 - protein, 9 aa, Artificial Sequence
  Non-human like sequence
NKKFPRGQN

SEQ ID NO: 532 - protein, 9 aa, Artificial Sequence
  Non-human like sequence
NKKFPRGQP

SEQ ID NO: 533 - protein, 9 aa, Artificial Sequence
  Non-human like sequence
NKKFPRGQS

SEQ ID NO: 534 - protein, 9 aa, Artificial Sequence
  Non-human like sequence
NLKFPRGQG

SEQ ID NO: 535 - protein, 9 aa, Artificial Sequence
  Non-human like sequence
NLKFPRGQL

SEQ ID NO: 536 - protein, 9 aa, Artificial Sequence
  Non-human like sequence
NLKFPRGQP

SEQ ID NO: 537 - protein, 9 aa, Artificial Sequence
  Non-human like sequence
NLKFPRGQS

SEQ ID NO: 538 - protein, 9 aa, Artificial Sequence
  Non-human like sequence
NSKFPRGQH

SEQ ID NO: 539 - protein, 9 aa, Artificial Sequence
  Non-human like sequence
NSKFPRGQL

SEQ ID NO: 540 - protein, 9 aa, Artificial Sequence
  Non-human like sequence
NSKFPRGQP

SEQ ID NO: 541 - protein, 9 aa, Artificial Sequence
  Non-human like sequence
NSKFPRGQS

SEQ ID NO: 542 - protein, 9 aa, Artificial Sequence
  Non-human like sequence
PCKFPRGQC

SEQ ID NO: 543 - protein, 9 aa, Artificial Sequence
  Non-human like sequence
PCKFPRGQG

SEQ ID NO: 544 - protein, 9 aa, Artificial Sequence
  Non-human like sequence
PCKFPRGQH

SEQ ID NO: 545 - protein, 9 aa, Artificial Sequence
  Non-human like sequence
PCKFPRGQL

SEQ ID NO: 546 - protein, 9 aa, Artificial Sequence
  Non-human like sequence
PCKFPRGQN

SEQ ID NO: 547 - protein, 9 aa, Artificial Sequence
  Non-human like sequence
PCKFPRGQP

SEQ ID NO: 548 - protein, 9 aa, Artificial Sequence
  Non-human like sequence
PCKFPRGQS

SEQ ID NO: 549 - protein, 9 aa, Artificial Sequence
  Non-human like sequence
PDKFPRGQP

SEQ ID NO: 550 - protein, 9 aa, Artificial Sequence
  Non-human like sequence
PHKFPRGQH

SEQ ID NO: 551 - protein, 9 aa, Artificial Sequence
  Non-human like sequence
PKKFPRGQG

SEQ ID NO: 552 - protein, 9 aa, Artificial Sequence
  Non-human like sequence
PKKFPRGQH

SEQ ID NO: 553 - protein, 9 aa, Artificial Sequence
  Non-human like sequence
PKKFPRGQL

SEQ ID NO: 554 - protein, 9 aa, Artificial Sequence
  Non-human like sequence
PKKFPRGQN

SEQ ID NO: 555 - protein, 9 aa, Artificial Sequence
  Non-human like sequence
PKKFPRGQP

SEQ ID NO: 556 - protein, 9 aa, Artificial Sequence
  Non-human like sequence
PKKFPRGQS

SEQ ID NO: 557 - protein, 9 aa, Artificial Sequence
  Non-human like sequence
PLKFPRGQL

SEQ ID NO: 558 - protein, 9 aa, Artificial Sequence
  Non-human like sequence
PLKFPRGQN

SEQ ID NO: 559 - protein, 9 aa, Artificial Sequence
  Non-human like sequence
PLKFPRGQP

SEQ ID NO: 560 - protein, 9 aa, Artificial Sequence
  Non-human like sequence
PLKFPRGQS

SEQ ID NO: 561 - protein, 9 aa, Artificial Sequence
  Non-human like sequence
QAKFPRGQC

SEQ ID NO: 562 - protein, 9 aa, Artificial Sequence
  Non-human like sequence
QAKFPRGQH

SEQ ID NO: 563 - protein, 9 aa, Artificial Sequence
  Non-human like sequence
QAKFPRGQL

SEQ ID NO: 564 - protein, 9 aa, Artificial Sequence
  Non-human like sequence
QAKFPRGQS

SEQ ID NO: 565 - protein, 9 aa, Artificial Sequence
  Non-human like sequence
QDKFPRGQL

SEQ ID NO: 566 - protein, 9 aa, Artificial Sequence
  Non-human like sequence
QQKFPRGQG

SEQ ID NO: 567 - protein, 9 aa, Artificial Sequence
  Non-human like sequence
QQKFPRGQH

SEQ ID NO: 568 - protein, 9 aa, Artificial Sequence
  Non-human like sequence
QQKFPRGQN

SEQ ID NO: 569 - protein, 9 aa, Artificial Sequence
  Non-human like sequence
QQKFPRGQP

SEQ ID NO: 570 - protein, 9 aa, Artificial Sequence
  Non-human like sequence
QQKFPRGQS

SEQ ID NO: 571 - protein, 9 aa, Artificial Sequence
  Non-human like sequence
SAKFPRGQC

SEQ ID NO: 572 - protein, 9 aa, Artificial Sequence
  Non-human like sequence
SAKFPRGQG

SEQ ID NO: 573 - protein, 9 aa, Artificial Sequence
  Non-human like sequence
SAKFPRGQH

SEQ ID NO: 574 - protein, 9 aa, Artificial Sequence
  Non-human like sequence
SAKFPRGQL

SEQ ID NO: 575 - protein, 9 aa, Artificial Sequence
  Non-human like sequence
SAKFPRGQP

SEQ ID NO: 576 - protein, 9 aa, Artificial Sequence
  Non-human like sequence
SAKFPRGQS

SEQ ID NO: 577 - protein, 9 aa, Artificial Sequence
  Non-human like sequence
SLKFPRGQG

SEQ ID NO: 578 - protein, 9 aa, Artificial Sequence
  Non-human like sequence
SLKFPRGQL

SEQ ID NO: 579 - protein, 9 aa, Artificial Sequence
  Non-human like sequence
SLKFPRGQP

SEQ ID NO: 580 - protein, 9 aa, Artificial Sequence
  Non-human like sequence
SLKFPRGQS

SEQ ID NO: 581 - protein, 9 aa, Artificial Sequence
  Non-human like sequence
TLKFPRGQL

SEQ ID NO: 582 - protein, 9 aa, Artificial Sequence
  Non-human like sequence
TLKFPRGQN

SEQ ID NO: 583 - protein, 9 aa, Artificial Sequence
  Non-human like sequence
TLKFPRGQP

SEQ ID NO: 584 - protein, 9 aa, Artificial Sequence
  Non-human like sequence
TLKFPRGQS

SEQ ID NO: 585 - protein, 9 aa, Artificial Sequence
  Non-human like sequence
TQKFPRGQP

SEQ ID NO: 586 - protein, 9 aa, Artificial Sequence
  Non-human like sequence
TQKFPRGQQ

SEQ ID NO: 587 - protein, 9 aa, Artificial Sequence
  Non-human like sequence
VAKFPRGQH

SEQ ID NO: 588 - protein, 9 aa, Artificial Sequence
  Non-human like sequence
VAKFPRGQL

SEQ ID NO: 589 - protein, 9 aa, Artificial Sequence
  Non-human like sequence
VAKFPRGQP

SEQ ID NO: 590 - protein, 9 aa, Artificial Sequence
  Non-human like sequence
VAKFPRGQS

SEQ ID NO: 591 - protein, 9 aa, Artificial Sequence
  Non-human like sequence
VSKFPRGQH

SEQ ID NO: 592 - protein, 9 aa, Artificial Sequence
  Non-human like sequence
VSKFPRGQL

SEQ ID NO: 593 - protein, 9 aa, Artificial Sequence
  Non-human like sequence
VSKFPRGQP

SEQ ID NO: 594 - protein, 9 aa, Artificial Sequence
  Non-human like sequence
VSKFPRGQQ

SEQ ID NO: 595 - protein, 9 aa, Artificial Sequence
  Non-human like sequence
VSKFPRGQS

SEQ ID NO: 596 - protein, 9 aa, Artificial Sequence
  Non-human like sequence
YAKFPRGQC

SEQ ID NO: 597 - protein, 9 aa, Artificial Sequence
  Non-human like sequence
YAKFPRGQH

SEQ ID NO: 598 - protein, 9 aa, Artificial Sequence
  Non-human like sequence
YAKFPRGQL

SEQ ID NO: 599 - protein, 9 aa, Artificial Sequence
  Non-human like sequence
YAKFPRGQP

SEQ ID NO: 600 - protein, 9 aa, Artificial Sequence
  Non-human like sequence
YAKFPRGQS

SEQ ID NO: 601 - protein, 9 aa, Artificial Sequence
  Non-human like sequence
YDKFPRGQL

SEQ ID NO: 602 - protein, 9 aa, Artificial Sequence
  Non-human like sequence
YDKFPRGQP

SEQ ID NO: 603 - protein, 9 aa, Artificial Sequence
  Non-human like sequence
YDKFPRGQS

SEQ ID NO: 604 - protein, 9 aa, Artificial Sequence
  Non-human like sequence
YKKFPRGQH

SEQ ID NO: 605 - protein, 9 aa, Artificial Sequence
  Non-human like sequence
YKKFPRGQL

SEQ ID NO: 606 - protein, 9 aa, Artificial Sequence
  Non-human like sequence
YKKFPRGQN

SEQ ID NO: 607 - protein, 9 aa, Artificial Sequence
  Non-human like sequence
YKKFPRGQP

SEQ ID NO: 608 - protein, 9 aa, Artificial Sequence
  Non-human like sequence
YKKFPRGQS

SEQ ID NO: 609 - protein, 9 aa, Artificial Sequence
  Non-human like sequence
YQKFPRGQP

SEQ ID NO: 610 - protein, 9 aa, Artificial Sequence
  Non-human like sequence
YQKFPRGQS

SEQ ID NO: 611 - protein, 9 aa, Artificial Sequence
  Non-human like sequence
YSKFPRGQH

SEQ ID NO: 612 - protein, 9 aa, Artificial Sequence
  Non-human like sequence
YSKFPRGQL

SEQ ID NO: 613 - protein, 9 aa, Artificial Sequence
  Non-human like sequence
YSKFPRGQS

SEQ ID NO: 614 - protein, 9 aa, Artificial Sequence
  Non-human like sequence
AGFPRGQGC

SEQ ID NO: 615 - protein, 9 aa, Artificial Sequence
  Non-human like sequence
AGFPRGQGD

SEQ ID NO: 616 - protein, 9 aa, Artificial Sequence
  Non-human like sequence
AGFPRGQGE

SEQ ID NO: 617 - protein, 9 aa, Artificial Sequence
  Non-human like sequence
AGFPRGQGG

SEQ ID NO: 618 - protein, 9 aa, Artificial Sequence
  Non-human like sequence
AGFPRGQGI

SEQ ID NO: 619 - protein, 9 aa, Artificial Sequence
  Non-human like sequence
AGFPRGQGK

SEQ ID NO: 620 - protein, 9 aa, Artificial Sequence
  Non-human like sequence
AGFPRGQGN

SEQ ID NO: 621 - protein, 9 aa, Artificial Sequence
  Non-human like sequence
AGFPRGQGQ

SEQ ID NO: 622 - protein, 9 aa, Artificial Sequence
  Non-human like sequence
AKFPRGQGN

SEQ ID NO: 623 - protein, 9 aa, Artificial Sequence
  Non-human like sequence
ASFPRGQGC

SEQ ID NO: 624 - protein, 9 aa, Artificial Sequence
  Non-human like sequence
CGFPRGQGC

SEQ ID NO: 625 - protein, 9 aa, Artificial Sequence
  Non-human like sequence
CGFPRGQGD

SEQ ID NO: 626 - protein, 9 aa, Artificial Sequence
  Non-human like sequence
CGFPRGQGE

SEQ ID NO: 627 - protein, 9 aa, Artificial Sequence
  Non-human like sequence
CGFPRGQGF

SEQ ID NO: 628 - protein, 9 aa, Artificial Sequence
  Non-human like sequence
CGFPRGQGG

SEQ ID NO: 629 - protein, 9 aa, Artificial Sequence
  Non-human like sequence
CGFPRGQGI

SEQ ID NO: 630 - protein, 9 aa, Artificial Sequence
  Non-human like sequence
CGFPRGQGK

SEQ ID NO: 631 - protein, 9 aa, Artificial Sequence
  Non-human like sequence
CGFPRGQGN

SEQ ID NO: 632 - protein, 9 aa, Artificial Sequence
  Non-human like sequence
CGFPRGQGQ

SEQ ID NO: 633 - protein, 9 aa, Artificial Sequence
  Non-human like sequence
CVFPRGQGC

SEQ ID NO: 634 - protein, 9 aa, Artificial Sequence
  Non-human like sequence
CVFPRGQGD

SEQ ID NO: 635 - protein, 9 aa, Artificial Sequence
  Non-human like sequence
CVFPRGQGE

SEQ ID NO: 636 - protein, 9 aa, Artificial Sequence
  Non-human like sequence
CVFPRGQGN

SEQ ID NO: 637 - protein, 9 aa, Artificial Sequence
  Non-human like sequence
CVFPRGQGQ

SEQ ID NO: 638 - protein, 9 aa, Artificial Sequence
  Non-human like sequence
DKFPRGQGG

SEQ ID NO: 639 - protein, 9 aa, Artificial Sequence
  Non-human like sequence
DKFPRGQGQ

SEQ ID NO: 640 - protein, 9 aa, Artificial Sequence
  Non-human like sequence
DVFPRGQGD

SEQ ID NO: 641 - protein, 9 aa, Artificial Sequence
  Non-human like sequence
DVFPRGQGF

SEQ ID NO: 642 - protein, 9 aa, Artificial Sequence
  Non-human like sequence
DVFPRGQGN

SEQ ID NO: 643 - protein, 9 aa, Artificial Sequence
  Non-human like sequence
DVFPRGQGQ

SEQ ID NO: 644 - protein, 9 aa, Artificial Sequence
  Non-human like sequence
EQFPRGQGD

SEQ ID NO: 645 - protein, 9 aa, Artificial Sequence
  Non-human like sequence
EQFPRGQGG

SEQ ID NO: 646 - protein, 9 aa, Artificial Sequence
  Non-human like sequence
EQFPRGQGK

SEQ ID NO: 647 - protein, 9 aa, Artificial Sequence
  Non-human like sequence
EQFPRGQGN

SEQ ID NO: 648 - protein, 9 aa, Artificial Sequence
  Non-human like sequence
FEFPRGQGI

SEQ ID NO: 649 - protein, 9 aa, Artificial Sequence
  Non-human like sequence
FQFPRGQGD

SEQ ID NO: 650 - protein, 9 aa, Artificial Sequence
  Non-human like sequence
FQFPRGQGE

SEQ ID NO: 651 - protein, 9 aa, Artificial Sequence
  Non-human like sequence
FQFPRGQGF

SEQ ID NO: 652 - protein, 9 aa, Artificial Sequence
  Non-human like sequence
FQFPRGQGG

SEQ ID NO: 653 - protein, 9 aa, Artificial Sequence
  Non-human like sequence
FQFPRGQGI

SEQ ID NO: 654 - protein, 9 aa, Artificial Sequence
  Non-human like sequence
FQFPRGQGK

SEQ ID NO: 655 - protein, 9 aa, Artificial Sequence
  Non-human like sequence
FQFPRGQGN

SEQ ID NO: 656 - protein, 9 aa, Artificial Sequence
  Non-human like sequence
FVFPRGQGC

SEQ ID NO: 657 - protein, 9 aa, Artificial Sequence
  Non-human like sequence
FVFPRGQGD

SEQ ID NO: 658 - protein, 9 aa, Artificial Sequence
  Non-human like sequence
FVFPRGQGE

SEQ ID NO: 659 - protein, 9 aa, Artificial Sequence
  Non-human like sequence
FVFPRGQGN

SEQ ID NO: 660 - protein, 9 aa, Artificial Sequence
  Non-human like sequence
FVFPRGQGQ

SEQ ID NO: 661 - protein, 9 aa, Artificial Sequence
  Non-human like sequence
GDFPRGQGE

SEQ ID NO: 662 - protein, 9 aa, Artificial Sequence
  Non-human like sequence
GDFPRGQGF

SEQ ID NO: 663 - protein, 9 aa, Artificial Sequence
  Non-human like sequence
GDFPRGQGG

SEQ ID NO: 664 - protein, 9 aa, Artificial Sequence
  Non-human like sequence
GDFPRGQGK

SEQ ID NO: 665 - protein, 9 aa, Artificial Sequence
  Non-human like sequence
GDFPRGQGN

SEQ ID NO: 666 - protein, 9 aa, Artificial Sequence
  Non-human like sequence
GDFPRGQGQ

SEQ ID NO: 667 - protein, 9 aa, Artificial Sequence
  Non-human like sequence
GDFPRGQGV

SEQ ID NO: 668 - protein, 9 aa, Artificial Sequence
  Non-human like sequence
GEFPRGQGA

SEQ ID NO: 669 - protein, 9 aa, Artificial Sequence
  Non-human like sequence
GEFPRGQGC

SEQ ID NO: 670 - protein, 9 aa, Artificial Sequence
  Non-human like sequence
GEFPRGQGD

SEQ ID NO: 671 - protein, 9 aa, Artificial Sequence
  Non-human like sequence
GEFPRGQGE

SEQ ID NO: 672 - protein, 9 aa, Artificial Sequence
  Non-human like sequence
GEFPRGQGF

SEQ ID NO: 673 - protein, 9 aa, Artificial Sequence
  Non-human like sequence
GEFPRGQGG

SEQ ID NO: 674 - protein, 9 aa, Artificial Sequence
  Non-human like sequence
GEFPRGQGI

SEQ ID NO: 675 - protein, 9 aa, Artificial Sequence
  Non-human like sequence
GEFPRGQGK

SEQ ID NO: 676 - protein, 9 aa, Artificial Sequence
  Non-human like sequence
GEFPRGQGN

SEQ ID NO: 677 - protein, 9 aa, Artificial Sequence
  Non-human like sequence
GEFPRGQGQ

SEQ ID NO: 678 - protein, 9 aa, Artificial Sequence
  Non-human like sequence
GEFPRGQGV

SEQ ID NO: 679 - protein, 9 aa, Artificial Sequence
  Non-human like sequence
GGFPRGQGE

SEQ ID NO: 680 - protein, 9 aa, Artificial Sequence
  Non-human like sequence
GGFPRGQGG

SEQ ID NO: 681 - protein, 9 aa, Artificial Sequence
  Non-human like sequence
GGFPRGQGK

SEQ ID NO: 682 - protein, 9 aa, Artificial Sequence
  Non-human like sequence
GGFPRGQGN

SEQ ID NO: 683 - protein, 9 aa, Artificial Sequence
  Non-human like sequence
GGFPRGQGQ

SEQ ID NO: 684 - protein, 9 aa, Artificial Sequence
  Non-human like sequence
GRFPRGQGD

SEQ ID NO: 685 - protein, 9 aa, Artificial Sequence
  Non-human like sequence
GRFPRGQGE

SEQ ID NO: 686 - protein, 9 aa, Artificial Sequence
  Non-human like sequence
GRFPRGQGQ

SEQ ID NO: 687 - protein, 9 aa, Artificial Sequence
  Non-human like sequence
GRFPRGQGV

SEQ ID NO: 688 - protein, 9 aa, Artificial Sequence
  Non-human like sequence
GSFPRGQGC

SEQ ID NO: 689 - protein, 9 aa, Artificial Sequence
  Non-human like sequence
GSFPRGQGG

SEQ ID NO: 690 - protein, 9 aa, Artificial Sequence
  Non-human like sequence
GSFPRGQGQ

SEQ ID NO: 691 - protein, 9 aa, Artificial Sequence
  Non-human like sequence
GSFPRGQGV

SEQ ID NO: 692 - protein, 9 aa, Artificial Sequence
  Non-human like sequence
HEFPRGQGE

SEQ ID NO: 693 - protein, 9 aa, Artificial Sequence
  Non-human like sequence
HEFPRGQGQ

SEQ ID NO: 694 - protein, 9 aa, Artificial Sequence
  Non-human like sequence
HQFPRGQGD

SEQ ID NO: 695 - protein, 9 aa, Artificial Sequence
  Non-human like sequence
HQFPRGQGE

SEQ ID NO: 696 - protein, 9 aa, Artificial Sequence
  Non-human like sequence
HQFPRGQGG

SEQ ID NO: 697 - protein, 9 aa, Artificial Sequence
  Non-human like sequence
HQFPRGQGI

SEQ ID NO: 698 - protein, 9 aa, Artificial Sequence
  Non-human like sequence
HQFPRGQGK

SEQ ID NO: 699 - protein, 9 aa, Artificial Sequence
  Non-human like sequence
IDFPRGQGF

SEQ ID NO: 700 - protein, 9 aa, Artificial Sequence
  Non-human like sequence
IDFPRGQGG

SEQ ID NO: 701 - protein, 9 aa, Artificial Sequence
  Non-human like sequence
IDFPRGQGQ

SEQ ID NO: 702 - protein, 9 aa, Artificial Sequence
  Non-human like sequence
IDFPRGQGV

SEQ ID NO: 703 - protein, 9 aa, Artificial Sequence
  Non-human like sequence
KKFPRGQGN

SEQ ID NO: 704 - protein, 9 aa, Artificial Sequence
  Non-human like sequence
LKFPRGQGN

SEQ ID NO: 705 - protein, 9 aa, Artificial Sequence
  Non-human like sequence
LKFPRGQGQ

SEQ ID NO: 706 - protein, 9 aa, Artificial Sequence
  Non-human like sequence
QKFPRGQGD

SEQ ID NO: 707 - protein, 9 aa, Artificial Sequence
  Non-human like sequence
QKFPRGQGE

SEQ ID NO: 708 - protein, 9 aa, Artificial Sequence
  Non-human like sequence
QKFPRGQGG

SEQ ID NO: 709 - protein, 9 aa, Artificial Sequence
  Non-human like sequence
QKFPRGQGN

SEQ ID NO: 710 - protein, 9 aa, Artificial Sequence
  Non-human like sequence
QKFPRGQGQ

SEQ ID NO: 711 - protein, 9 aa, Artificial Sequence
  Non-human like sequence
QKFPRGQGV

SEQ ID NO: 712 - protein, 9 aa, Artificial Sequence
  Non-human like sequence
SSFPRGQGG

SEQ ID NO: 713 - protein, 9 aa, Artificial Sequence
  Non-human like sequence
SSFPRGQGQ

SEQ ID NO: 714 - protein, 9 aa, Artificial Sequence
  Non-human like sequence
SSFPRGQGV

SEQ ID NO: 715 - protein, 9 aa, Artificial Sequence
  Non-human like sequence
SVFPRGQGE

SEQ ID NO: 716 - protein, 9 aa, Artificial Sequence
  Non-human like sequence
VDFPRGQGF

SEQ ID NO: 717 - protein, 9 aa, Artificial Sequence
  Non-human like sequence
VDFPRGQGG

SEQ ID NO: 718 - protein, 9 aa, Artificial Sequence
  Non-human like sequence
VDFPRGQGQ

SEQ ID NO: 719 - protein, 9 aa, Artificial Sequence
  Non-human like sequence
VDFPRGQGV

SEQ ID NO: 720 - protein, 9 aa, Artificial Sequence
  Non-human like sequence
VEFPRGQGA

SEQ ID NO: 721 - protein, 9 aa, Artificial Sequence
  Non-human like sequence
VEFPRGQGD

SEQ ID NO: 722 - protein, 9 aa, Artificial Sequence
  Non-human like sequence
VEFPRGQGE

SEQ ID NO: 723 - protein, 9 aa, Artificial Sequence
  Non-human like sequence
VEFPRGQGG

SEQ ID NO: 724 - protein, 9 aa, Artificial Sequence
  Non-human like sequence
VEFPRGQGI

SEQ ID NO: 725 - protein, 9 aa, Artificial Sequence
  Non-human like sequence
VEFPRGQGK

SEQ ID NO: 726 - protein, 9 aa, Artificial Sequence
  Non-human like sequence
VEFPRGQGN

SEQ ID NO: 727 - protein, 9 aa, Artificial Sequence
  Non-human like sequence
VEFPRGQGQ

SEQ ID NO: 728 - protein, 9 aa, Artificial Sequence
  Non-human like sequence
VEFPRGQGR

SEQ ID NO: 729 - protein, 9 aa, Artificial Sequence
  Non-human like sequence
VEFPRGQGV

SEQ ID NO: 730 - protein, 9 aa, Artificial Sequence
  Non-human like sequence
VGFPRGQGC

SEQ ID NO: 731 - protein, 9 aa, Artificial Sequence
  Non-human like sequence
VGFPRGQGD

SEQ ID NO: 732 - protein, 9 aa, Artificial Sequence
  Non-human like sequence
VGFPRGQGE

SEQ ID NO: 733 - protein, 9 aa, Artificial Sequence
  Non-human like sequence
VGFPRGQGG

SEQ ID NO: 734 - protein, 9 aa, Artificial Sequence
  Non-human like sequence
VGFPRGQGI

SEQ ID NO: 735 - protein, 9 aa, Artificial Sequence
  Non-human like sequence
VGFPRGQGK

SEQ ID NO: 736 - protein, 9 aa, Artificial Sequence
  Non-human like sequence
VGFPRGQGN

SEQ ID NO: 737 - protein, 9 aa, Artificial Sequence
  Non-human like sequence
VGFPRGQGQ

SEQ ID NO: 738 - protein, 9 aa, Artificial Sequence
  Non-human like sequence
VSFPRGQGC

SEQ ID NO: 739 - protein, 9 aa, Artificial Sequence
  Non-human like sequence
VSFPRGQGG

SEQ ID NO: 740 - protein, 9 aa, Artificial Sequence
  Non-human like sequence
WGFPRGQGC

SEQ ID NO: 741 - protein, 9 aa, Artificial Sequence
  Non-human like sequence
WGFPRGQGD

SEQ ID NO: 742 - protein, 9 aa, Artificial Sequence
  Non-human like sequence
WGFPRGQGE

SEQ ID NO: 743 - protein, 9 aa, Artificial Sequence
  Non-human like sequence
WGFPRGQGG

SEQ ID NO: 744 - protein, 9 aa, Artificial Sequence
  Non-human like sequence
WGFPRGQGI

SEQ ID NO: 745 - protein, 9 aa, Artificial Sequence
  Non-human like sequence
WGFPRGQGQ

SEQ ID NO: 746 - protein, 9 aa, Artificial Sequence
  Non-human like sequence
WYFPRGQGD

SEQ ID NO: 747 - protein, 9 aa, Artificial Sequence
  Non-human like sequence
WYFPRGQGE

SEQ ID NO: 748 - protein, 9 aa, Artificial Sequence
  Non-human like sequence
WYFPRGQGG

SEQ ID NO: 749 - protein, 9 aa, Artificial Sequence
  Non-human like sequence
WYFPRGQGI

SEQ ID NO: 750 - protein, 9 aa, Artificial Sequence
  Non-human like sequence
WYFPRGQGV

SEQ ID NO: 751 - protein, 9 aa, Artificial Sequence
  Non-human like sequence
YEFPRGQGD

SEQ ID NO: 752 - protein, 9 aa, Artificial Sequence
  Non-human like sequence
YEFPRGQGE

SEQ ID NO: 753 - protein, 9 aa, Artificial Sequence
  Non-human like sequence
YEFPRGQGI

SEQ ID NO: 754 - protein, 9 aa, Artificial Sequence
  Non-human like sequence
YEFPRGQGN

SEQ ID NO: 755 - protein, 9 aa, Artificial Sequence
  Non-human like sequence
YEFPRGQGQ

SEQ ID NO: 756 - protein, 9 aa, Artificial Sequence
  Non-human like sequence
DFPRGQGVE

SEQ ID NO: 757 - protein, 9 aa, Artificial Sequence
  Non-human like sequence
DFPRGQGVG

SEQ ID NO: 758 - protein, 9 aa, Artificial Sequence
  Non-human like sequence
DFPRGQGVI

SEQ ID NO: 759 - protein, 9 aa, Artificial Sequence
  Non-human like sequence
DFPRGQGVM

SEQ ID NO: 760 - protein, 9 aa, Artificial Sequence
  Non-human like sequence
DFPRGQGVQ

SEQ ID NO: 761 - protein, 9 aa, Artificial Sequence
  Non-human like sequence
DHPRGQGVM

SEQ ID NO: 762 - protein, 9 aa, Artificial Sequence
  Non-human like sequence
DHPRGQGVQ

SEQ ID NO: 763 - protein, 9 aa, Artificial Sequence
  Non-human like sequence
EFPRGQGVE

SEQ ID NO: 764 - protein, 9 aa, Artificial Sequence
  Non-human like sequence
EFPRGQGVF

SEQ ID NO: 765 - protein, 9 aa, Artificial Sequence
  Non-human like sequence
EFPRGQGVG

SEQ ID NO: 766 - protein, 9 aa, Artificial Sequence
  Non-human like sequence
EFPRGQGVI

SEQ ID NO: 767 - protein, 9 aa, Artificial Sequence
  Non-human like sequence
EFPRGQGVM

SEQ ID NO: 768 - protein, 9 aa, Artificial Sequence
  Non-human like sequence
EFPRGQGVQ

SEQ ID NO: 769 - protein, 9 aa, Artificial Sequence
  Non-human like sequence
ETPRGQGVG

SEQ ID NO: 770 - protein, 9 aa, Artificial Sequence
  Non-human like sequence
ETPRGQGVQ

SEQ ID NO: 771 - protein, 9 aa, Artificial Sequence
  Non-human like sequence
FYPRGQGVF

SEQ ID NO: 772 - protein, 9 aa, Artificial Sequence
  Non-human like sequence
FYPRGQGVG

SEQ ID NO: 773 - protein, 9 aa, Artificial Sequence
  Non-human like sequence
FYPRGQGVI

SEQ ID NO: 774 - protein, 9 aa, Artificial Sequence
  Non-human like sequence
FYPRGQGVQ

SEQ ID NO: 775 - protein, 9 aa, Artificial Sequence
  Non-human like sequence
GVPRGQGVE

SEQ ID NO: 776 - protein, 9 aa, Artificial Sequence
  Non-human like sequence
GYPRGQGVF

SEQ ID NO: 777 - protein, 9 aa, Artificial Sequence
  Non-human like sequence
GYPRGQGVG

SEQ ID NO: 778 - protein, 9 aa, Artificial Sequence
  Non-human like sequence
GYPRGQGVI

SEQ ID NO: 779 - protein, 9 aa, Artificial Sequence
  Non-human like sequence
HHPRGQGVM

SEQ ID NO: 780 - protein, 9 aa, Artificial Sequence
  Non-human like sequence
HYPRGQGVE

SEQ ID NO: 781 - protein, 9 aa, Artificial Sequence
  Non-human like sequence
KFPRGQGVQ

SEQ ID NO: 782 - protein, 9 aa, Artificial Sequence
  Non-human like sequence
KYPRGQGVE

SEQ ID NO: 783 - protein, 9 aa, Artificial Sequence
  Non-human like sequence
LTPRGQGVG

SEQ ID NO: 784 - protein, 9 aa, Artificial Sequence
  Non-human like sequence
LTPRGQGVM

SEQ ID NO: 785 - protein, 9 aa, Artificial Sequence
  Non-human like sequence
LTPRGQGVQ

SEQ ID NO: 786 - protein, 9 aa, Artificial Sequence
  Non-human like sequence
MAPRGQGVQ

SEQ ID NO: 787 - protein, 9 aa, Artificial Sequence
  Non-human like sequence
PYPRGQGVF

SEQ ID NO: 788 - protein, 9 aa, Artificial Sequence
  Non-human like sequence
PYPRGQGVI

SEQ ID NO: 789 - protein, 9 aa, Artificial Sequence
  Non-human like sequence
QHPRGQGVG

SEQ ID NO: 790 - protein, 9 aa, Artificial Sequence
  Non-human like sequence
QHPRGQGVM

SEQ ID NO: 791 - protein, 9 aa, Artificial Sequence
  Non-human like sequence
QHPRGQGVQ

SEQ ID NO: 792 - protein, 9 aa, Artificial Sequence
  Non-human like sequence
QYPRGQGVE

SEQ ID NO: 793 - protein, 9 aa, Artificial Sequence
  Non-human like sequence
QYPRGQGVF

SEQ ID NO: 794 - protein, 9 aa, Artificial Sequence
  Non-human like sequence
QYPRGQGVG

SEQ ID NO: 795 - protein, 9 aa, Artificial Sequence
  Non-human like sequence
QYPRGQGVI

SEQ ID NO: 796 - protein, 9 aa, Artificial Sequence
  Non-human like sequence
QYPRGQGVQ

SEQ ID NO: 797 - protein, 9 aa, Artificial Sequence
  Non-human like sequence
RFPRGQGVA

SEQ ID NO: 798 - protein, 9 aa, Artificial Sequence
  Non-human like sequence
RFPRGQGVE

SEQ ID NO: 799 - protein, 9 aa, Artificial Sequence
  Non-human like sequence
RFPRGQGVF

SEQ ID NO: 800 - protein, 9 aa, Artificial Sequence
  Non-human like sequence
RFPRGQGVG

SEQ ID NO: 801 - protein, 9 aa, Artificial Sequence
  Non-human like sequence
RFPRGQGVI

SEQ ID NO: 802 - protein, 9 aa, Artificial Sequence
  Non-human like sequence
RFPRGQGVM

SEQ ID NO: 803 - protein, 9 aa, Artificial Sequence
  Non-human like sequence
RFPRGQGVQ

SEQ ID NO: 804 - protein, 9 aa, Artificial Sequence
  Non-human like sequence
SAPRGQGVM

SEQ ID NO: 805 - protein, 9 aa, Artificial Sequence
  Non-human like sequence
SAPRGQGVQ

SEQ ID NO: 806 - protein, 9 aa, Artificial Sequence
  Non-human like sequence
SFPRGQGVE

SEQ ID NO: 807 - protein, 9 aa, Artificial Sequence
  Non-human like sequence
SFPRGQGVG

SEQ ID NO: 808 - protein, 9 aa, Artificial Sequence
  Non-human like sequence
STPRGQGVM

SEQ ID NO: 809 - protein, 9 aa, Artificial Sequence
  Non-human like sequence
STPRGQGVN

SEQ ID NO: 810 - protein, 9 aa, Artificial Sequence
  Non-human like sequence
STPRGQGVQ

SEQ ID NO: 811 - protein, 9 aa, Artificial Sequence
  Non-human like sequence
TAPRGQGVM

SEQ ID NO: 812 - protein, 9 aa, Artificial Sequence
  Non-human like sequence
TAPRGQGVQ

SEQ ID NO: 813 - protein, 9 aa, Artificial Sequence
  Non-human like sequence
TTPRGQGVQ

SEQ ID NO: 814 - protein, 9 aa, Artificial Sequence
  Non-human like sequence
TVPRGQGVE

SEQ ID NO: 815 - protein, 9 aa, Artificial Sequence
  Non-human like sequence
VHPRGQGVM

SEQ ID NO: 816 - protein, 9 aa, Artificial Sequence
  Non-human like sequence
VTPRGQGVM

SEQ ID NO: 817 - protein, 9 aa, Artificial Sequence
  Non-human like sequence
VTPRGQGVQ

SEQ ID NO: 818 - protein, 9 aa, Artificial Sequence
  Non-human like sequence
WYPRGQGVF

SEQ ID NO: 819 - protein, 9 aa, Artificial Sequence
  Non-human like sequence
WYPRGQGVG

SEQ ID NO: 820 - protein, 9 aa, Artificial Sequence
  Non-human like sequence
WYPRGQGVI

SEQ ID NO: 821 - protein, 9 aa, Artificial Sequence
  Non-human like sequence
YFPRGQGVE

SEQ ID NO: 822 - protein, 9 aa, Artificial Sequence
  Non-human like sequence
YFPRGQGVG

SEQ ID NO: 823 - protein, 9 aa, Artificial Sequence
  Non-human like sequence
YFPRGQGVI

SEQ ID NO: 824 - protein, 9 aa, Artificial Sequence
  Non-human like sequence
YFPRGQGVQ

SEQ ID NO: 825 - protein, 9 aa, Artificial Sequence
  Non-human like sequence
AKRGQGVPT

SEQ ID NO: 826 - protein, 9 aa, Artificial Sequence
  Non-human like sequence
AMRGQGVPG

SEQ ID NO: 827 - protein, 9 aa, Artificial Sequence
  Non-human like sequence
AMRGQGVPH

SEQ ID NO: 828 - protein, 9 aa, Artificial Sequence
  Non-human like sequence
AMRGQGVPS

SEQ ID NO: 829 - protein, 9 aa, Artificial Sequence
  Non-human like sequence
HMRGQGVPT

SEQ ID NO: 830 - protein, 9 aa, Artificial Sequence
  Non-human like sequence
IQRGQGVPS

SEQ ID NO: 831 - protein, 9 aa, Artificial Sequence
  Non-human like sequence
LQRGQGVPS

SEQ ID NO: 832 - protein, 9 aa, Artificial Sequence
  Non-human like sequence
PKRGQGVPC

SEQ ID NO: 833 - protein, 9 aa, Artificial Sequence
  Non-human like sequence
PKRGQGVPT

SEQ ID NO: 834 - protein, 9 aa, Artificial Sequence
  Non-human like sequence
SKRGQGVPT

SEQ ID NO: 835 - protein, 9 aa, Artificial Sequence
  Non-human like sequence
TKRGQGVPC

SEQ ID NO: 836 - protein, 9 aa, Artificial Sequence
  Non-human like sequence
TKRGQGVPH

SEQ ID NO: 837 - protein, 9 aa, Artificial Sequence
  Non-human like sequence
TKRGQGVPT

SEQ ID NO: 838 - protein, 9 aa, Artificial Sequence
  Non-human like sequence
AGPINTNSA

SEQ ID NO: 839 - protein, 9 aa, Artificial Sequence
  Non-human like sequence
HGPINTNSA

SEQ ID NO: 840 - protein, 9 aa, Artificial Sequence
  Non-human like sequence
HGPINTNSL

SEQ ID NO: 841 - protein, 9 aa, Artificial Sequence
  Non-human like sequence
LRPINTNSL

SEQ ID NO: 842 - protein, 9 aa, Artificial Sequence
  Non-human like sequence
MAPINTNSA

SEQ ID NO: 843 - protein, 9 aa, Artificial Sequence
  Non-human like sequence
MGPINTNSA

SEQ ID NO: 844 - protein, 9 aa, Artificial Sequence
  Non-human like sequence
QGPINTNSL

SEQ ID NO: 845 - protein, 9 aa, Artificial Sequence
  Non-human like sequence
RRPINTNSL

SEQ ID NO: 846 - protein, 9 aa, Artificial Sequence
  Non-human like sequence
DGTRRIRGK

SEQ ID NO: 847 - protein, 9 aa, Artificial Sequence
  Non-human like sequence
DGTRRIRGQ

SEQ ID NO: 848 - protein, 9 aa, Artificial Sequence
  Non-human like sequence
HGTRRIRGK

SEQ ID NO: 849 - protein, 9 aa, Artificial Sequence
  Non-human like sequence
HGTRRIRGQ

SEQ ID NO: 850 - protein, 9 aa, Artificial Sequence
  Non-human like sequence
IKTRRIRGQ

SEQ ID NO: 851 - protein, 9 aa, Artificial Sequence
  Non-human like sequence
KGTRRIRGQ

SEQ ID NO: 852 - protein, 9 aa, Artificial Sequence
  Non-human like sequence
PTTRRIRGK

SEQ ID NO: 853 - protein, 9 aa, Artificial Sequence
  Non-human like sequence
PTTRRIRGQ

SEQ ID NO: 854 - protein, 9 aa, Artificial Sequence
  Non-human like sequence
PTTRRIRGT

SEQ ID NO: 855 - protein, 9 aa, Artificial Sequence
  Non-human like sequence
DKIRGGDGM

SEQ ID NO: 856 - protein, 9 aa, Artificial Sequence
  Non-human like sequence
GKIRGGDGM

SEQ ID NO: 857 - protein, 9 aa, Artificial Sequence
  Non-human like sequence
HKIRGGDGM

SEQ ID NO: 858 - protein, 9 aa, Artificial Sequence
  Non-human like sequence
QKIRGGDGM

SEQ ID NO: 859 - protein, 9 aa, Artificial Sequence
  Non-human like sequence
AHRGGDGKD

SEQ ID NO: 860 - protein, 9 aa, Artificial Sequence
  Non-human like sequence
AHRGGDGKE

SEQ ID NO: 861 - protein, 9 aa, Artificial Sequence
  Non-human like sequence
AHRGGDGKF

SEQ ID NO: 862 - protein, 9 aa, Artificial Sequence
  Non-human like sequence
AHRGGDGKS

SEQ ID NO: 863 - protein, 9 aa, Artificial Sequence
  Non-human like sequence
AKRGGDGKE

SEQ ID NO: 864 - protein, 9 aa, Artificial Sequence
  Non-human like sequence
AKRGGDGKF

SEQ ID NO: 865 - protein, 9 aa, Artificial Sequence
  Non-human like sequence
AKRGGDGKK

SEQ ID NO: 866 - protein, 9 aa, Artificial Sequence
  Non-human like sequence
AKRGGDGKP

SEQ ID NO: 867 - protein, 9 aa, Artificial Sequence
  Non-human like sequence
AMRGGDGKP

SEQ ID NO: 868 - protein, 9 aa, Artificial Sequence
  Non-human like sequence
ATRGGDGKE

SEQ ID NO: 869 - protein, 9 aa, Artificial Sequence
  Non-human like sequence
CCRGGDGKP

SEQ ID NO: 870 - protein, 9 aa, Artificial Sequence
  Non-human like sequence
CLRGGDGKE

SEQ ID NO: 871 - protein, 9 aa, Artificial Sequence
  Non-human like sequence
GKRGGDGKD

SEQ ID NO: 872 - protein, 9 aa, Artificial Sequence
  Non-human like sequence
GKRGGDGKE

SEQ ID NO: 873 - protein, 9 aa, Artificial Sequence
  Non-human like sequence
GKRGGDGKF

SEQ ID NO: 874 - protein, 9 aa, Artificial Sequence
  Non-human like sequence
GKRGGDGKK

SEQ ID NO: 875 - protein, 9 aa, Artificial Sequence
  Non-human like sequence
GKRGGDGKP

SEQ ID NO: 876 - protein, 9 aa, Artificial Sequence
  Non-human like sequence
HCRGGDGKP

SEQ ID NO: 877 - protein, 9 aa, Artificial Sequence
  Non-human like sequence
HKRGGDGKD

SEQ ID NO: 878 - protein, 9 aa, Artificial Sequence
  Non-human like sequence
HKRGGDGKE

SEQ ID NO: 879 - protein, 9 aa, Artificial Sequence
  Non-human like sequence
HKRGGDGKF

SEQ ID NO: 880 - protein, 9 aa, Artificial Sequence
  Non-human like sequence
HKRGGDGKP

SEQ ID NO: 881 - protein, 9 aa, Artificial Sequence
  Non-human like sequence
HTRGGDGKD

SEQ ID NO: 882 - protein, 9 aa, Artificial Sequence
  Non-human like sequence
HTRGGDGKE

SEQ ID NO: 883 - protein, 9 aa, Artificial Sequence
  Non-human like sequence
HTRGGDGKF

SEQ ID NO: 884 - protein, 9 aa, Artificial Sequence
  Non-human like sequence
HTRGGDGKK

SEQ ID NO: 885 - protein, 9 aa, Artificial Sequence
  Non-human like sequence
HTRGGDGKP

SEQ ID NO: 886 - protein, 9 aa, Artificial Sequence
  Non-human like sequence
HTRGGDGKS

SEQ ID NO: 887 - protein, 9 aa, Artificial Sequence
  Non-human like sequence
ICRGGDGKD

SEQ ID NO: 888 - protein, 9 aa, Artificial Sequence
  Non-human like sequence
ICRGGDGKE

SEQ ID NO: 889 - protein, 9 aa, Artificial Sequence
  Non-human like sequence
ITRGGDGKD

SEQ ID NO: 890 - protein, 9 aa, Artificial Sequence
  Non-human like sequence
ITRGGDGKE

SEQ ID NO: 891 - protein, 9 aa, Artificial Sequence
  Non-human like sequence
ITRGGDGKF

SEQ ID NO: 892 - protein, 9 aa, Artificial Sequence
  Non-human like sequence
ITRGGDGKK

SEQ ID NO: 893 - protein, 9 aa, Artificial Sequence
  Non-human like sequence
ITRGGDGKP

SEQ ID NO: 894 - protein, 9 aa, Artificial Sequence
  Non-human like sequence
ITRGGDGKS

SEQ ID NO: 895 - protein, 9 aa, Artificial Sequence
  Non-human like sequence
KCRGGDGKP

SEQ ID NO: 896 - protein, 9 aa, Artificial Sequence
  Non-human like sequence
KLRGGDGKE

SEQ ID NO: 897 - protein, 9 aa, Artificial Sequence
  Non-human like sequence
LHRGGDGKE

SEQ ID NO: 898 - protein, 9 aa, Artificial Sequence
  Non-human like sequence
NKRGGDGKD

SEQ ID NO: 899 - protein, 9 aa, Artificial Sequence
  Non-human like sequence
NKRGGDGKE

SEQ ID NO: 900 - protein, 9 aa, Artificial Sequence
  Non-human like sequence
NKRGGDGKR

SEQ ID NO: 901 - protein, 9 aa, Artificial Sequence
  Non-human like sequence
NLRGGDGKE

SEQ ID NO: 902 - protein, 9 aa, Artificial Sequence
  Non-human like sequence
QKRGGDGKP

SEQ ID NO: 903 - protein, 9 aa, Artificial Sequence
  Non-human like sequence
SHRGGDGKD

SEQ ID NO: 904 - protein, 9 aa, Artificial Sequence
  Non-human like sequence
SHRGGDGKE

SEQ ID NO: 905 - protein, 9 aa, Artificial Sequence
  Non-human like sequence
SHRGGDGKF

SEQ ID NO: 906 - protein, 9 aa, Artificial Sequence
  Non-human like sequence
SHRGGDGKS

SEQ ID NO: 907 - protein, 9 aa, Artificial Sequence
  Non-human like sequence
THRGGDGKD

SEQ ID NO: 908 - protein, 9 aa, Artificial Sequence
  Non-human like sequence
THRGGDGKF

SEQ ID NO: 909 - protein, 9 aa, Artificial Sequence
  Non-human like sequence
TKRGGDGKD

SEQ ID NO: 910 - protein, 9 aa, Artificial Sequence
  Non-human like sequence
TKRGGDGKE

SEQ ID NO: 911 - protein, 9 aa, Artificial Sequence
  Non-human like sequence
TKRGGDGKF

SEQ ID NO: 912 - protein, 9 aa, Artificial Sequence
  Non-human like sequence
TKRGGDGKK

SEQ ID NO: 913 - protein, 9 aa, Artificial Sequence
  Non-human like sequence
TKRGGDGKP

SEQ ID NO: 914 - protein, 9 aa, Artificial Sequence
  Non-human like sequence
TKRGGDGKR

SEQ ID NO: 915 - protein, 9 aa, Artificial Sequence
  Non-human like sequence
TTRGGDGKD

SEQ ID NO: 916 - protein, 9 aa, Artificial Sequence
  Non-human like sequence
TTRGGDGKE

SEQ ID NO: 917 - protein, 9 aa, Artificial Sequence
  Non-human like sequence
TTRGGDGKF

SEQ ID NO: 918 - protein, 9 aa, Artificial Sequence
  Non-human like sequence
TTRGGDGKP

SEQ ID NO: 919 - protein, 9 aa, Artificial Sequence
  Non-human like sequence
TTRGGDGKS

SEQ ID NO: 920 - protein, 9 aa, Artificial Sequence
  Non-human like sequence
VTRGGDGKD

SEQ ID NO: 921 - protein, 9 aa, Artificial Sequence
  Non-human like sequence
VTRGGDGKF

SEQ ID NO: 922 - protein, 9 aa, Artificial Sequence
  Non-human like sequence
VTRGGDGKK

SEQ ID NO: 923 - protein, 9 aa, Artificial Sequence
  Non-human like sequence
VTRGGDGKP

SEQ ID NO: 924 - protein, 9 aa, Artificial Sequence
  Non-human like sequence
VTRGGDGKS

SEQ ID NO: 925 - protein, 9 aa, Artificial Sequence
  Non-human like sequence
WCRGGDGKD

SEQ ID NO: 926 - protein, 9 aa, Artificial Sequence
  Non-human like sequence
WCRGGDGKE

SEQ ID NO: 927 - protein, 9 aa, Artificial Sequence
  Non-human like sequence
WCRGGDGKP

SEQ ID NO: 928 - protein, 13 aa, SARS-CoV-2
PCNGVEGFNCYFP

SEQ ID NO: 929 - protein, 9 aa, SARS-CoV-2
LDSKVGGNY

SEQ ID NO: 930 - protein, 11 aa, SARS-CoV-2
FQPTNGVGYQP

SEQ ID NO: 931 - protein, 14 aa, Artificial Sequence
  Non-human like sequence
FKCYGVSPTKLNDS

SEQ ID NO: 932 - protein, 11 aa, SARS-CoV-2
PLSETKCTLKS

SEQ ID NO: 933 - protein, 19 aa, Artificial Sequence
  Non-human like sequence
PATVCGPKKSTNLVKNKCV

SEQ ID NO: 934 - protein, 26 aa, Artificial Sequence
  Non-human like sequence
RGPCNGVEGRGTPCNGVGRGGVEGFN

SEQ ID NO: 935 - protein, 19 aa, Artificial Sequence
  Non-human like sequence
RGKVGGNYGRGDSKVGGRG

SEQ ID NO: 936 - protein, 26 aa, Artificial Sequence
  Non-human like sequence
RGTNGVGYGRGFQPTNGGRGGVGYQP

SEQ ID NO: 937 - protein, 26 aa, Artificial Sequence
  Non-human like sequence
RGCYGVSPGRGFKCYGVGRGVSPTKL

SEQ ID NO: 938 - protein, 26 aa, Artificial Sequence
  Non-human like sequence
RGPLSETKGRGKCTLKSGRGSETKCT

SEQ ID NO: 939 - protein, 26 aa, Artificial Sequence
  Non-human like sequence
RGTVCGPKGRGPKKSTNGRGVKNKCV

SEQ ID NO: 940 - protein, 20 aa, Artificial Sequence
  Non-human like sequence
RGKVGGNYQNRLDSKVGGRN

SEQ ID NO: 941 - protein, 35 aa, Artificial Sequence
  Non-human like sequence
RRGPCNGVENRTPSNGVENRNGVEGFNNRSTPSNG

SEQ ID NO: 942 - protein, 28 aa, Artificial Sequence
  Non-human like sequence
RRRGSTPCNGVEGFQSNGVEGFNCWQRR

SEQ ID NO: 943 - protein, 28 aa, Artificial Sequence
  Non-human like sequence
RGTNGVGYNNRFQPTNGRNRGVGYQPRN

SEQ ID NO: 944 - protein, 29 aa, Artificial Sequence
  Non-human like sequence
RGASTEKSNRNGINITRQRRLLHAPATVG

SEQ ID NO: 945 - protein, 35 aa, Artificial Sequence
  Non-human like sequence
RRGFKSYGVSPTKLNDSKVGGNYQNRLDSKVGGNY

SEQ ID NO: 946 - protein, 36 aa, Artificial Sequence
  Non-human like sequence
RRSTPSNGVERRGVEGFNENRFQPTNGRNRGVGYQP

SEQ ID NO: 947 - protein, 35 aa, Artificial Sequence
  Non-human like sequence
RRGASTEKSNRNGINITRQLLHAPATVRTNGVGYG

SEQ ID NO: 948 - protein, 33 aa, Artificial Sequence
  Non-human like sequence
RRKSTNLVGGATVTGPGGGVKNKSVGGPLSETK

SEQ ID NO: 949 - protein, 33 aa, Artificial Sequence
  Non-human like sequence
RRKSTNLVGGQLTPTWGGGVKNKSVGGPLSETK

SEQ ID NO: 950 - protein, 36 aa, Artificial Sequence
  Non-human like sequence
RRFYPRGQGVFLEGATNTASWFRSRGFQFPRGQGIG

SEQ ID NO: 951 - protein, 51 aa, Artificial Sequence
  Non-human like sequence
RNGVKGFNCYFCLQSYGPTYGVGYQPNNLDSKVGGNYLYCRLFRYKGTQGR

SEQ ID NO: 952 - protein, 32 aa, Artificial Sequence
  Non-human like sequence
QTQTNGSQSIIAGCGNLTTRTQKRFANGATWC

SEQ ID NO: 953 - protein, 25 aa, Artificial Sequence
  Non-human like sequence
DCEGKYHKNNKSWCEAVHRSYITPG

SEQ ID NO: 954 - protein, 32 aa, Artificial Sequence
  Non-human like sequence
TVRDPQTCDITESNKKFIPLGCGQLTPTWGRR

SEQ ID NO: 955 - protein, 19 aa, Artificial Sequence
  Segment of SeqIDNo1
HRSYLTPGDSSSGWTAGAA

SEQ ID NO: 956 - protein, 23 aa, Artificial Sequence
  Segment of SeqIDNo1
VYYHKNNKSWMESEFRVYSSANN

SEQ ID NO: 957 - protein, 22 aa, Artificial Sequence
  Segment of SeqIDNo1
ISTEIYQAGSTPCNGVEGFNCY

SEQ ID NO: 958 - protein, 14 aa, Artificial Sequence
  Segment of SeqIDNo1
TQTNSPRRARSVAS

SEQ ID NO: 959 - protein, 14 aa, Artificial Sequence
  Segment of SeqIDNo1
TQTNSPSGAGSVAS

SEQ ID NO: 960 - protein, 12 aa, Artificial Sequence
  Segment of SeqIDNo1
HVSGTNGTKRFD